(19) United States
(12) Patent Application Publication
(10) Pub. No.: US 2026/0315828 A1
(43) Pub. Date: Oct. 15, 2026

(54) METHODS FOR IMAGE-BASED DOSE DELIVERY FOR RADIOTHERAPY

(71) Applicant: RefleXion Medical, Inc., Hayward, CA (US)

(72) Inventors: Harshali BAL, Fremont, CA (US); Samuel BRYANT, Los Angeles, CA (US); Charles Wenpeng HUANG, Santa Clara, CA (US); Jeffrey Peter SCHMALL, Pleasant Hill, CA (US); Shiyu XU, San Ramon, CA (US)

(21) Appl. No.: 19/644,606

(22) Filed: Apr. 10, 2026

Related U.S. Application Data
(60) Provisional application No. 63/786,786, filed on Apr. 10, 2025.
(60) Provisional application No. 63/992,455, filed on Feb. 27, 2026.

(51) Int. Cl. A61N 5/10; G06T 7/246; G06T 7/277; G06T 7/70

(52) U.S. Cl. CPC A61N 5/1049; A61N 5/1039; A61N 5/1071; G06T 7/246; G06T 7/277; G06T 7/70; A61N 2005/1052; G06T 2207/10104; G06T 2207/20048; G06T 2207/20076; G06T 2207/20081; G06T 2207/20216; G06T 2207/30096

(57) ABSTRACT

Disclosed herein are methods for image-based dose delivery, which includes estimating the location of a tumor during a treatment session using imaging data acquired in real-time. One method for calculating a tumor location includes merging multiple methods of estimating the location of a tumor based on imaging data using a Kalman filter. The tumor location is used to generate an anchor point image. The delivery fluence is then derived from the anchor point image. Methods for estimating the location of a target during a treatment session may include a statistical inference approach, adaptive correlation filtering, and/or centroid tracking. Also disclosed herein are methods for deblurring PET images of a moving tumor target using principal component analysis and generating a treatment plan for biology-guided radiotherapy using deblurred PET images.

### CROSS-REFERENCE TO RELATED APPLICATIONS

This application claims priority to U.S. Provisional Patent Application No. 63/786,786 filed Apr. 10, 2025, and U.S. Provisional Patent Application No. 63/992,455 filed Feb. 27, 2026, the disclosures of which are hereby incorporated in their entirety.

### BACKGROUND

External beam radiotherapy aims to deliver a lethal dose of radiation to one or more tumors while sparing healthy tissues surrounding the tumor(s). One challenge to effective radiation delivery is the result of tumor motion. Most, if not all, tumors are subject to motion, whether expected or unexpected, intentional or unintentional. This makes it difficult for a radiotherapy system to ensure that radiation beams are always delivered to the tumors as intended. External beam radiotherapy aims to deliver a lethal dose of radiation to one or more tumors while sparing healthy tissues surrounding the tumor(s). One challenge to effective radiation delivery is the result of tumor motion. Most, if not all, tumors are subject to motion, whether expected or unexpected, intentional or unintentional. This makes it difficult for a radiotherapy system to ensure that radiation beams are always delivered to the tumors as intended.

Since the location of a tumor may change, one approach has been to irradiate its entire likely motion path, and therefore, ensure that the prescribed dose is delivered to the tumor regardless of its motion. However, this irradiates a substantial portion of healthy tissue surrounding the tumor. Biology-guided radiotherapy seeks to address this issue by using PET emissions from a tumor to direct beamlets of external radiotherapy back to the tumor with sub-second latency. While PET imaging data that is rapidly acquired during a treatment session provides real-time information about the tumor, the shortened image acquisition time intervals may result in images with artifacts and/or noise, which may result in a less than optimal dose delivery.

Accordingly, improved approaches to delivering radiation dose to tumors are desired.

### SUMMARY

Disclosed herein are methods of estimating the location of a tumor during a treatment session using imaging data acquired in real-time for delivering a radiation dose that is more conformal to the size and shape of the tumor. One variation of a method for calculating a tumor location includes merging multiple methods of estimating the location of a tumor based on imaging data using a Kalman filter. The delivery fluence is then derived from and/or shifted to the calculated tumor location. Methods for estimating the location of a target during a treatment session may include a statistical inference approach, adaptive correlation filtering, and/or centroid tracking. Each method for estimating the location of the target may include a position estimation confidence number and/or a confidence interval that may be used by the Kalman filter to merge the location estimates and calculate the tumor location. Also disclosed herein are methods for deblurring PET images of a moving tumor target using principal component analysis and generating a treatment plan for biology-guided radiotherapy using deblurred PET images.

One variation of a method for calculating a tumor location for improving radiation dose conformality to a target region may comprise acquiring imaging data of a target region using image detectors, analyzing the imaging data using a first algorithm to generate a first estimated location of the target region, analyzing the imaging data using a second algorithm to generate a second estimated location of the target region, wherein the second algorithm is different from the first algorithm, generating a final location of the target region by combining the first estimated location and the second estimated location, and calculating a radiation fluence according to the final location of the target region, where the calculated radiation fluence, when emitted, delivers a conformal radiation dose to the target region. In some variations, generating the final location of the target region may comprise combining the first estimated location and the second estimated location using a Kalman filter. The method may further comprise analyzing the imaging data using a third algorithm to generate a third estimated location of the target region. Generating the final location of the target region may comprise combining the first estimated location, the second estimated location, and the third estimated location. For example, generating the final location of the target region may comprise combining the first estimated location, the second estimated location, and the third estimated location using a Kalman filter. Analyzing the imaging data using the first algorithm may further comprise calculating a first confidence value associated with the first estimated location, and analyzing the imaging data using the second algorithm may further comprise calculating a second confidence value associated with the second estimated location. Generating the final location of the target region may comprise weighting the first estimated location by the first confidence value, weighting the second estimated location by the second confidence value, and combining the weighted first estimated location and the weighted second estimated location.

In some variations, using the first algorithm may comprise applying a correlation filter to the imaging data to generate the first estimated location of the target region and using the second algorithm may comprise performing a maximum likelihood estimation analysis on the imaging data to generate the second estimated location of the target region. Alternatively, or additionally, using the first algorithm may comprise applying a correlation filter to the imaging data to generate the first estimated location of the target region and using the second algorithm may comprise calculating a location of a centroid of the imaging data to generate the second estimated location of the target region. In some variations, the correlation filter may be an adaptive correlation filter that is trained on images of the target region and locations of the target region in the images. Alternatively, or additionally, using the first algorithm may comprise performing a maximum likelihood estimation analysis on the imaging data to generate the first estimated location of the target region and using the second algorithm may comprise calculating a location of a centroid of the imaging data to generate the second estimated location of the target region. In some variations, the imaging data may comprise time-of-flight PET imaging data, and the first algorithm may comprise calculating a location of a centroid using the time-of-flight PET imaging data to generate the first estimated location of the target region. The method may comprise generating a notification that indicates the final location of the target region.

In some variations, the imaging data may comprise positron annihilation emission path data, and the image detectors may comprise PET detectors. Optionally, the imaging data may comprise time-of-flight (TOF) positron annihilation emission path data. Using the first algorithm may comprise calculating a location of a centroid of the TOF positron annihilation emission path data by determining a region where the TOF positron annihilation emission paths intersect. In some variations, acquiring imaging data may comprise acquiring imaging data from the image detectors for a first time duration, and the first algorithm may generate the first estimated location using the imaging data acquired during a second time duration that is a subset within the first time duration and the second algorithm may generate the second estimated location using the imaging data acquired during the first time duration. As an example, the second estimated location may be a running average of the target region location during the first time duration, and the first estimated location may be the location of the target region during the second time interval. In some instances, the first time duration may be from about 3 seconds to about 6 seconds, and the second time duration may be from about 200 ms to about 600 ms. Combining the first estimated location and the second estimated location may comprise correcting a bias of the first estimated location using the second estimated location. Alternatively, or additionally, combining the first estimated location and the second estimated location may comprise correcting a bias of the second estimated location using the first estimated location. In some variations, the final location of the target region may include spatial coordinates of the target region, e.g., 3-D coordinates. Alternatively, or additionally, calculating the radiation fluence for delivery may comprise adjusting a planned fluence map according to the final location of the target region, for example, shifting the planned fluence map to the final location of the target region. In some variations, calculating the radiation fluence for delivery may comprise generating an image of the final location of the target region and convolving the generated image of the final location with a firing filter. Acquiring the imaging data of the target region and calculating the radiation fluence for delivery may both occur in a single radiation delivery session and/or acquiring the imaging data of the target region and calculating the radiation fluence for delivery may both occur in real-time. Alternatively, or additionally, calculating the radiation fluence for delivery may occur within 1 second of acquiring the imaging data of the target region, e.g., 500 ms of acquiring the imaging data of the target region. Optionally, the method may further comprise delivering the calculated radiation fluence to the final location of the target region.

Also disclosed herein are statistical inference methods that may be used to estimate tumor location(s) from low-photon count images. One variation of a method is a maximum likelihood estimate method, which may comprise acquiring a low-photon count image of a target region, where the target region has an initial location, applying a plurality of probability density functions corresponding to a plurality of target region shifts to the low-photon count image to calculate a likelihood value for each of the plurality of probability density functions, determining a target region shift by identifying the target region shift that corresponds to a maximum likelihood value from the likelihood values for the plurality of probability functions, and calculating an updated location of the target region by applying the determined target region shift to the initial location of the target region. The plurality of probability density functions may be generated by shifting a nominal probability density function by the plurality of target region shifts. In one example, the nominal probability density function may be derived from a high-photon count image of the target region location, e.g., the high-photon count image of the target region may comprise one or more of positron annihilation emission path imaging data acquired during treatment planning, positron annihilation emission path imaging data acquired during a treatment session, and/or positron annihilation emission path imaging data acquired during a radiation delivery session. Optionally, the nominal probability density function may incorporate known and/or expected variabilities and/or errors of the location estimation and/or measurement imprecisions. In some variations, the low-photon count images may comprise one or more of positron annihilation emission path data acquired during an acquisition time interval that is less than 10 seconds, and/or lines-of-response (LORs) data measured by PET detectors. Optionally, the method may comprise generating an additional probability density function for a previously-determined target region shift. For example, generating the additional probability density function may comprise applying a Bayesian model to the updated target location that has been shifted by a previously-determined target region shift.

Disclosed herein are methods of reducing or removing motion artifacts from PET images for treatment planning. One variation of a method for generating a reduced-motion PET image of a target region may comprise decomposing PET imaging data of a target region into a set of principal components using principal component analysis, identifying a principal component from the set of principal components that has a dominant frequency similar to a predetermined frequency range, defining a set of motion phases using the identified principal component, grouping the PET imaging data into its corresponding motion phase within the set of motion phases, generating phase-gated PET images by summing the PET imaging data at each motion phase, aligning each of the phase-gated PET images with a reference target location, and summing the phase-gated PET images to generate a reduced-motion image of the target region. The method may optionally comprise generating a radiotherapy treatment plan using the reduced-motion image of the target region. The radiotherapy treatment plan may comprise a planned radiation fluence map to deliver a prescribed radiation dose to the target region. Aligning each of the phase-gated PET images with the reference target location may comprise determining a shift vector for each of the phase-gated PET images by comparing the phase-gated PET images with the reference target region location, and shifting each of the phase-gated PET images according to the shift vector, which may, in some instances, be a 3-D shift vector. In some variations, the set of principal components may comprise 3 or more principal components. The method may optionally comprise acquiring PET imaging data of the target region using PET detectors. The predetermined frequency range may be approximately a frequency range of human respiration and/or of human peristaltic motion. The predetermined frequency range may be approximately a frequency range of about 0.05 Hz to about 0.6 Hz.

Some methods may further comprise applying a difference of Gaussians filter to the PET imaging data before decomposing the PET imaging data into the set of principal components. The PET imaging data may comprise lines-of-response data. The lines-of-response data may be acquired using PET detectors within a specified acquisition time interval, e.g., 5 seconds or less, 1 second or less, 500 ms or less. In some variations, the PET imaging data may comprise time-of-flight PET data. Identifying the principal component that has a dominant frequency similar to the predetermined frequency range may comprise applying a Fourier transform to the set of principal components. Defining the set of motion phases may comprise applying a Hilbert transform to the identified principal component. In some variations, the set of motion phases may comprise 16 phases. Optionally, a method may comprise determining a phase-specific location of the target region for each phase of the set of motion phases based on the phase-gated PET images, and calculating a motion vector for each group of PET imaging data based on the phase-specific location of the target region in the set of motion phases.

Disclosed herein are methods for generating a PET image training (e.g., initializing) data set for testing target tracking methods. One variation of this method may comprise decomposing PET imaging data of a target region into a set of principal components using principal component analysis, identifying a principal component from the set of principal components that has a dominant frequency similar to a predetermined frequency range, defining a set of motion phases using the identified principal component, grouping the PET imaging data into its corresponding motion phase within the set of motion phases, generating phase-gated PET images by summing the PET imaging data at each motion phase, determining a target region location for each motion phase by comparing the phase-gated PET images to a reference target location, and generating a training data set comprising the PET imaging data for each motion phase and the determined target region location associated with that motion phase. The PET imaging data may comprise PET lines-of-response (LOR) data, and the training data set may comprise PET LOR data for each motion phase and the determined target region location associated with that motion phase. Alternatively, or additionally, the PET imaging data may comprise PET imaging data acquired during a series of acquisition time intervals, and the training data set may comprise PET imaging data acquired during specific acquisition time intervals within a motion phase and the determined target region location associated with that motion phase. In some variations, the set of principal components may comprise 3 or more principal components. The method may optionally comprise acquiring PET imaging data of the target region using PET detectors. The predetermined frequency range may be approximately a frequency range of human respiration and/or of human peristaltic motion. The predetermined frequency range may be approximately a frequency range of about 0.05 Hz to about 0.6 Hz. In some variations, the PET imaging data may comprise lines-of-response data. The lines-of-response data may be acquired using PET detectors within a specified acquisition time interval, e.g., 5 seconds or less, 1 second or less, 500 ms or less. In some variations, the PET imaging data may comprise time-of-flight PET data. Identifying the principal component that has a dominant frequency similar to the predetermined frequency range may comprise applying a Fourier transform to the set of principal components. Defining the set of motion phases may comprise applying a Hilbert transform to the identified principal component. In some variations, the set of motion phases may comprise 16 phases.

Another variation of a method for reducing or removing motion artifacts in PET images for treatment planning may use contours within the PET images. One variation of a method may comprise calculating a background level of PET activity in a first PET image of a tumor, wherein the tumor has a tumor contour and an initial level of PET activity within the tumor contour, defining a set of PET activity isocontours in the first PET image within a predefined contour that encompasses the tumor, where the isocontours delineate a corresponding set of isocontour shells, calculating a tumor PET activity for each isocontour shell, calculating a total tumor PET activity within the predefined contour by summing the tumor PET activity in the set of isocontour shells, generating a background image by setting a PET activity for the set of isocontour shells to a background level of PET activity, adjusting the initial level of PET activity within the tumor contour to have the calculated total tumor PET activity, and generating a second PET image by combining the tumor contour with the adjusted level of PET activity and the background image. The method may further comprise generating a radiotherapy treatment plan using the second PET image. The predefined contour may be a biology-tracking zone (BTZ) contour. The method may further comprise calculating the background level of PET activity by calculating PET activity in a region outside of the predefined contour, e.g., the region outside of the predefined contour may be a shell region that is an expansion of the predefined contour. Optionally, the method may comprise calculating the background level of PET activity by subtracting the calculated tumor PET activity for an isocontour shell from the PET activity for the isocontour shell. Adjusting the initial level of PET activity within the tumor contour may comprise scaling the initial level of PET activity such that a total PET activity within the tumor contour matches the calculated total tumor PET activity. Alternatively, or additionally, some variations may comprise calculating a scaling factor based on a ratio of the calculated total tumor PET activity to the initial level of PET activity, and adjusting the initial level of PET activity may comprise calculating a PET activity offset by multiplying pixel values within the tumor contour by the scaling factor and/or modulating the pixel values within the tumor contour by the PET activity offset. Alternatively, or additionally, adjusting the initial level of PET activity within the tumor contour may comprise normalizing the initial level of PET activity within the tumor contour to the total tumor PET activity.

In some variations, the method may optionally comprise acquiring a CT image of the tumor and defining the tumor contour using the CT image. The initial level of PET activity within the tumor contour may be derived by overlaying the CT image over the first PET image and calculating PET activity within the tumor contour. Some variations may comprise determining an isocontour PET activity for each isocontour shell based on pixel intensity values of the blurred PET image, and calculating the tumor PET activity for each isocontour shell may comprise subtracting the background level of PET activity from the isocontour PET activity. Alternatively, or additionally, some variations may comprise determining an isocontour PET activity for each isocontour shell based on pixel intensity values of the blurred PET image, and calculating the tumor PET activity for each isocontour shell may comprise scaling the isocontour PET activity by a factor derived from a ratio of the background level of PET activity to a PET activity of the isocontour.

Also disclosed herein are methods for radiotherapy treatment planning. Some variations of treatment planning use a location uncertainty model to create the treatment plan instead of an image of the target region. One variation of a method for treatment planning may comprise acquiring PET images of a target region, deriving a first set of target region locations corresponding to phases of a target region motion trajectory from the acquired PET images, deriving a second set of target region locations corresponding to phases of the target region motion trajectory by analyzing the acquired PET images using a location estimation algorithm, generating a location uncertainty model by determining differences between the first and second sets of target region locations for each phase of the target region motion trajectory, and generating a radiotherapy treatment plan comprising a set of firing filters iteratively calculated from the uncertainty model. The method may optionally comprise deblurring the acquired PET images to generate motion-corrected PET images and defining the phases of the target region motion trajectory using the motion-corrected PET images. Deriving the first set of target region locations may comprise aligning the motion-corrected PET images to a reference location of the target region. Optionally, the method may further comprise training the location estimation algorithm on the motion-corrected PET images. In some variations, deblurring the acquired PET images may comprise decomposing the PET images into a set of principal components and identifying a principal component from the set of principal components. Defining the phases of the target region motion trajectory may comprise defining the phases based on the identified principal component. In some variations, the location uncertainty model may comprise a location uncertainty cloud having a plurality of target region locations and/or a plurality of target region shifts from an initial target region location. Generating the radiotherapy treatment plan may comprise iterating through candidate firing filters such that when convolved with the location uncertainty model, the radiotherapy treatment plan attains a prescribed radiation dose to the target region while minimizing a dose cost function. In some variations, acquiring PET images of a target region may comprise acquiring PET images concurrently with acquiring target region position data using a position sensor, and deriving the first set of target region locations may comprise defining phases of the motion trajectory using the target region position data and binning the acquired PET images into the corresponding phase during which the PET images were acquired.

Disclosed herein are methods for generating a tracking uncertainty model. One variation of a method may comprise acquiring PET images of a target region over a plurality of motion phases, deblurring the target region images to obtain motion-corrected PET images, training a location estimation algorithm to identify a location of a target region in an image, applying the location estimation algorithm to the motion-corrected PET images to determine a set of estimated target region shifts, and generating a tracking uncertainty model from the set of estimated target region shifts. Training the location estimation algorithm may comprise using the motion-corrected PET images that have been aligned to a reference target region location. Optionally, the set of estimated target region shifts may be a first set of estimated target region shifts, and the method may further comprise applying the location estimation algorithm to the acquired PET images to determine a second set of estimated target region shifts. Generating the tracking uncertainty model may comprise computing differences between the first set of estimated target region shifts and the second set of estimated target region shifts. Training the location estimation algorithm may comprise using the acquired PET images.

Further disclosed herein are methods for evaluating the quality of a location estimation algorithm. One variation may comprise generating a first series of target region location data for multiple motion phases based on images of a moving target region, generating a second series of estimated target region location data for the multiple motion phases by applying a location estimation algorithm to the images of a moving target region, generating a location uncertainty model by comparing the first series of target region location data with the second series of target region location data, computing a characteristic of the location uncertainty model, evaluating the characteristic of the location uncertainty model against a predetermined acceptable range of that characteristic, and determining a quality of the location estimation algorithm based on whether the evaluated characteristic is within the acceptable range. In one variation, the location uncertainty model has a spread that may be defined as 2 times a standard deviation of the location uncertainty model, and evaluating the characteristic of the location uncertainty model may comprise comparing the spread against a threshold spread value. Determining the quality of the location estimation algorithm may comprise determining whether the spread is below the threshold spread value. For example, the threshold spread value may be about 20 mm or less along any dimension of the location uncertainty model, and determining the quality of the location estimation algorithm may comprise determining whether the spread is less than or equal to about 20 mm. Alternatively, or additionally, the location uncertainty model has a mean value, and evaluating the characteristic of the location uncertainty model may comprise comparing the mean value against a threshold mean value. Determining the quality of the location estimation algorithm may comprise determining whether the mean value is below the threshold mean value. For example, the threshold mean value may be about 3 mm or less, and determining the quality of the location estimation algorithm may comprise determining whether the mean value is less than or equal to about 3 mm. Alternatively, or additionally, generating a first series of target region location data for multiple motion phases based on images of a moving target region may comprise deblurring the images of the moving target region to obtain motion-corrected images, and the method may further comprise calculating a deblurring ratio by computing a ratio of an activity concentration of the target region in the motion-corrected images to an activity concentration of the target region in the images of the moving target region. Determining the quality of the location estimation algorithm may comprise determining whether the deblurring ratio is at least a threshold ratio value. For example, the threshold ratio value may be at least 0.8, e.g., 0.9 or more.

Also disclosed herein are methods for evaluating PET imaging data for radiation delivery. One variation of a method comprises acquiring PET prescan imaging data of a target region, initializing a location estimation algorithm using PET prescan images generated from the PET prescan imaging data, generating a location uncertainty model by applying the initialized location estimation algorithm to the PET imaging data, determining a difference between the location uncertainty model and an initial location uncertainty model generated using planning PET imaging data, and generating a notification that indicates whether the determined difference exceeds a threshold. If the determined difference does not exceed a threshold, the method further comprises delivering radiation to the target region, but if the determined difference exceeds a threshold, the method does not comprise delivering radiation to the target region. The notification may comprise a graphical user interface that depicts the determined difference and the threshold, and/or may further comprises a prompt for the user to exit radiation delivery. Acquiring PET prescan imaging data of the target region may comprise detecting positron annihilation emission paths using one or more PET detectors of a radiotherapy system. In one variation, the location estimation algorithm may comprise the use of an adaptive correlation filter, and initializing the location estimation algorithm may comprise generating a correlation filter using the PET prescan images. The location estimation algorithm may comprise the use of a maximum likelihood estimation method, and initializing the location estimation algorithm may comprise generating a plurality of probability density functions using the PET prescan images. Alternatively, or additionally, the location estimation algorithm may be a first location estimation algorithm and the method further comprises initializing a second location estimation algorithm, where the first location estimation algorithm may comprise the use of an adaptive correlation filter and the second location estimation algorithm may comprise the use of a maximum likelihood estimation method. Initializing the location estimation algorithm may comprise generating a correlation filter using the PET prescan images and generating a plurality of probability density functions using the PET prescan images.

In one variation, applying the initialized location estimation algorithm to the PET imaging data may generate a first series of target region location data, and generating a location uncertainty model may further comprise comparing the first series of target region location data with a second series of target region location data to determine a series of location differences, and generating a location uncertainty cloud from the series of location differences. Determining the difference between the location uncertainty model and an initial location uncertainty model may comprise determining a difference between a spread of each of the location uncertainty models, where the spread of a location uncertainty model may be 2 times a standard deviation of the location uncertainty model. If the difference in the uncertainty model spread exceeds 3 mm in any dimension [x,y,z], the generated notification may comprise a visual indicator that the difference in the uncertainty model exceeds the threshold. Alternatively, or additionally, determining the difference between the location uncertainty model and an initial location uncertainty model may comprise determining a difference between a mean of each of the location uncertainty models, where the mean of a location uncertainty model is an average of error values in location uncertainty model. If the difference in the uncertainty model spread exceeds 3 mm, the generated notification may comprise a visual indicator that the difference in the uncertainty model exceeds the threshold. Optionally, some methods may further comprise calculating a deblurring ratio by deblurring the PET prescan image of the target region to generate motion-corrected images, and computing a ratio of an activity concentration of the target region in the motion-corrected images to an activity concentration of the target region in the PET prescan images. The method may further comprise determining whether the deblurring ratio is at least a threshold ratio value, e.g., where the threshold deblurring ratio value is at least 0.8, or at least 0.9. if the deblurring ratio is not at or above the threshold ratio value, the generated notification may comprise a visual indicator that the deblurring ratio is below the threshold ratio value.

Also disclosed herein are methods for radiotherapy treatment planning. One variation of a method of radiotherapy treatment planning may comprise acquiring PET imaging data of a target region in a treatment position within an acquisition time window, initializing a location estimation algorithm using PET images reconstructed from the acquired PET imaging data, calculating a location uncertainty model for the initialized location estimation algorithm, and generating a radiotherapy treatment plan comprising a set of firing filters iteratively calculated from the location uncertainty model. The method may further comprise generating motion-corrected PET images by deblurring the reconstructed PET images. In some variations, deblurring the PET images may comprise decomposing the reconstructed PET images into a set of principal components and identifying a dominant principal component from the set of principal components. The location uncertainty model may comprise a location uncertainty cloud comprising a plurality of target region shifts from an initial target region location. In some variations, generating the radiotherapy treatment plan may comprise iterating through candidate firing filters such that when the firing filters are convolved with the location uncertainty model, the radiotherapy treatment plan attains a prescribed radiation dose to the target region while minimizing a dose cost function. The location estimation algorithm may comprise the use of an adaptive correlation filter, and initializing the location estimation algorithm may comprise generating a correlation filter using the motion-corrected PET image. The location estimation algorithm may comprise the use of a maximum likelihood estimation method, and initializing the location estimation algorithm may comprise generating a plurality of probability density functions using the motion-corrected PET images.

In some variations, the location uncertainty model may have a spread defined as 2 times a standard deviation of the location uncertainty model, and the method may further comprise determining whether the spread is below a threshold spread value, and proceeding to generate the radiotherapy treatment plan if the spread is less than or equal to the threshold spread value. The threshold spread value may be 20 mm or less along any dimension of the location uncertainty model. Alternatively, or additionally, the location uncertainty model may have a mean value, and the method may further comprise determining whether the mean value is below the threshold mean value, and proceeding to generate the radiotherapy treatment plan if the mean value is less than or equal to the threshold mean value. The threshold mean value may be 3 mm or less. Some variations of the method may comprise calculating a deblurring ratio by computing a ratio of an activity concentration of the target region in the motion-corrected PET images to an activity concentration of the target region in the reconstructed PET images. The method may further comprise determining whether the deblurring ratio is greater than or equal to a threshold ratio value. The threshold ratio value is greater than or equal to 0.8, e.g., greater than or equal to 0.9.

In some variations, initializing a location estimation algorithm using PET images reconstructed from the acquired PET imaging data may comprise applying filtered back projection to the acquired PET imaging data. Alternatively, or additionally, initializing a location estimation algorithm using PET images reconstructed from the acquired PET imaging data may comprise applying single-slice rebinning and filtered back projection to the acquired PET imaging data. Some variations may optionally comprise aligning the reconstructed PET images with a planning CT image, and/or aligning the motion-corrected PET images with a planning CT image, and/or aligning the reconstructed PET images with a planning MR image. Alternatively, or additionally, a method may further comprise computing an activity concentration (AC) of the target region, and proceeding to generate the radiotherapy treatment plan if the activity concentration is within an acceptable range, e.g., where the acceptable range of the activity concentration may be greater than or equal to 5 kBq/ml. Alternatively, or additionally, a method may comprise computing a normalized target signal (NTS) of the target region, and proceeding to generate the radiotherapy treatment plan if the normalized target signal is within an acceptable range, e.g., where the acceptable range of the normalized target signal may be greater than or equal to 2, greater than or equal to 2.7. In some variations, the radiotherapy treatment plan further comprises a planned fluence map that, when delivered, provides a prescribed dose to the target region.

### BRIEF DESCRIPTION OF THE DRAWINGS

FIG. 1A is a block diagram of one variation of radiotherapy system

FIG. 1B is a schematic perspective view of one variation of a radiotherapy system.

FIG. 2A is a flowchart representation of one variation of a method for generating an estimated location of a target region using imaging data acquired during the radiation delivery session and delivering radiation to the location of the target region.

FIG. 2B is a flowchart representation of one variation of a method for generating an estimated location of a target region and calculating a radiation fluence according to the estimated location.

FIG. 2C is a conceptual diagram depicting how acquired imaging data is analyzed using the methods of FIGS. 2A-2B.

FIG. 2D is conceptual diagram depicting a radiotherapy treatment planning and delivery workflow for determining real-time estimates of a target location.

FIG. 3A is a flowchart representation of one variation of a statistical inference method that may be used to estimate a location of a target region from low-photon imaging data.

FIG. 3B is a flowchart representation of one variation of a method for generating a plurality of probability density functions.

FIG. 3C is a flowchart representation of one variation of a statistical inference method for generating an estimated location of a target region using time-of-flight (TOF) PET data.

FIG. 3D depicts an example of a TOF PET backprojection image of 3 LORs.

FIG. 3E depicts an example of an image of LOR origin points of the 3 LORs depicted in FIG. 3D.

FIG. 3F is a flowchart representation of one variation of a statistical inference method for generating an estimated location of a target region images and/or PDFs in sinogram space.

FIG. 4 is a flowchart representation of one variation of a method that may be used to estimate a location of a target region using time-of-flight PET data.

FIG. 5A is a flowchart representation of one variation of a method for calculating a target anchor location and radiation fluence for delivery using an adaptive correlation filter.

FIG. 5B is a flowchart depiction of one variation of a method for calculating a fluence for delivery using the estimated location of a target region.

FIG. 5C is a flowchart representation of one variation of a method for generating an ACF based on planning CT images and planning PET images.

FIG. 5D is a flowchart representation of one variation of a method for updating the adaptive correlation filter.

FIG. 6A is a conceptual depiction of one variation of a Kalman filter that may be used to generate an estimated location of a target region.

FIG. 6B is a flowchart depiction of one variation of a method for combining a first estimated location and a second estimated location of a target region using a Kalman filter to generate a final estimated location of the target region.

FIG. 7A is a flowchart representation of one variation of a treatment planning method.

FIG. 7B is a flowchart representation of one variation of a method for biology-guided radiotherapy treatment planning using motion-reduced PET imaging data.

FIG. 7C depicts a conceptual diagram of a treatment planning method that uses a location uncertainty model to calculate a firing matrix (P) comprising a set of firing filters (pi).

FIG. 8A is a flowchart representation of one variation of a method for deblurring PET images.

FIG. 8B is a flowchart representation of one variation of a method for generating a training data set comprising PET imaging data and target region location for each motion phase of a target region.

FIG. 8C is a flowchart depiction of one variation of a method for generating a reduced-motion (e.g., “deblurred”) PET image.

FIG. 8D is a conceptual depiction of the various contours and shell regions used in the method of FIG. 8C.

FIG. 8E is a flowchart depiction of a variation of a method for generating a reduced-motion (e.g., “deblurred”) PET image.

FIG. 9A is a flowchart representation of one variation of a method for calculating a tracking error metric for a location estimator method or algorithm.

FIG. 9B is a flowchart representation of one variation of a method for calculating a tracking or location uncertainty model for one or more location estimator algorithms.

FIG. 9C is a flowchart representation of one variation of a method for calculating a tracking or location uncertainty model (e.g., uncertainty cloud) for one or more location estimator algorithms.

FIG. 10A is a flowchart representation of one variation of a method for calculating a tracking or location uncertainty model (e.g., uncertainty cloud) by shuffling (e.g., reassigning) detection time stamps for PET LORs.

FIG. 10B is a conceptual diagram of LTS images of a target region reconstructed from LORs and their original time stamps.

FIG. 10C is a conceptual diagram of LTS images of a target region reconstructed from LORs and their shuffled time stamps.

FIG. 10D is a conceptual diagram of LTS images reconstructed from PET LORs that have not been motion-corrected.

FIG. 10E is a conceptual diagram of LTS images reconstructed from PET LORs that have been at least partially motion-corrected.

FIG. 10F is a conceptual diagram of LTS images reconstructed from PET LORs that have been at least partially motion-corrected and their time stamps shuffled.

FIG. 11A depicts a first image LTS 1 (g₁) and which corresponds with Anchor 1 (g₁). FIG. 11D depicts the associated position shift of Anchor 1 (g₁), e.g., a first LTS image labeled with a first shift.

FIG. 11B depicts a second image LTS 2 (g₂) and which corresponds with Anchor 2 (g₂). FIG. 11E depicts the associated position shift of Anchor 2 (g₂), e.g., a second LTS image labeled with a second shift.

FIG. 11C depicts a third image LTS 3 (g₃) and which corresponds with Anchor 3 (g₃). FIG. 11F depicts the associated position shift of Anchor 3 (g₃), e.g., a third LTS image labeled with a third shift.

FIG. 12A is a conceptual depiction of a location estimation method using MLE with nearest neighbor weighted averaging.

FIG. 12B is a flowchart representation of one variation of a target location estimation method that uses nearest neighbor weighted averaging.

FIG. 13A is a flowchart representation of one variation of a method for radiotherapy treatment planning for PET-guided radiotherapy (e.g., biology-guided radiotherapy).

FIG. 13B is a flowchart representation of one variation of a method for the delivery of PET-guided radiotherapy (e.g., biology-guided radiotherapy).

### DETAILED DESCRIPTION

Disclosed herein are methods for improving the conformality of a delivered radiation dose to one or more target regions using imaging data acquired during the radiation dose delivery. The imaging data acquired during radiation dose delivery may be sparse or noisy, and the methods described herein may analyze the imaging data using a plurality of algorithms to estimate the location of a target region, and direct radiation precisely to the target region by converting an image of the target region location into a deliverable radiation fluence. One of the algorithms that may be used to calculate the location of a target region based on sparse or noisy image data may be a maximum likelihood estimation method that uses a plurality of probability density functions corresponding to a plurality of target region locations and/or shifts. Applying these probability density functions to imaging data acquired in real-time (e.g., by cross-correlating the imaging data with the probability density functions) may help estimate the location of the target region(s). Another variation of a method that may be used to calculate the location of a target region may use time-of-flight (TOF) PET data to determine the location of a centroid of a plurality of lines-of-response. An image of the location of a target region may help improve the precision and conformality of the radiation fluence calculated from the location. The image of the location of the target region may also facilitate the generation of a treatment plan fluence map that is more conformal and less susceptible to imaging data noise and artifacts.

The methods described herein may be used in the context of radiotherapy delivery that is guided by PET imaging data (e.g., PET emission data) acquired in real-time, during a treatment session. Real-time acquired imaging data is used to calculate a radiation fluence for delivering a prescribed dose to a target region, e.g., tumor. Delivering radiation within a short time period after acquiring the imaging data may help the radiotherapy system deliver radiation to the actual location of the tumor, instead of delivering radiation to a previous location of the tumor. One approach to this type of radiotherapy (which may be referred to as emission-guided radiotherapy or biology-guided radiotherapy) is to convert the imaging data into radiation fluence for delivery. However, in this approach, artifacts in imaging data may also be translated into artifacts in the delivered dose, for example, in implementations where the imaging data is linearly transformed into radiation dose. This may occur during treatment planning and/or during radiation delivery. In some circumstances, imaging data of non-tumor tissue (e.g., background imaging data or background noise) may be difficult to reduce or remove, which may result in some radiation dose being delivered to non-tumor tissue. With one or more of these artifacts at play, the result is that the delivered radiation dose may not be as conformal as initially intended, which may overdose healthy tissue, underdose the tumor, and/or create “hot spots” of radiation dose. These undesirable effects may be exacerbated if the tumor moves and/or if there are multiple tumors being treated in a single treatment session.

The methods described herein help improve dose conformality to the size and shape of the target region so that the prescribed radiation dose is delivered to the (moving) tumor (e.g., target region, planning target volume or PTV) and radiation is reduced to non-target tissue. In some variations, the methods may comprise determining the location of the tumor(s) during the radiotherapy treatment session using imaging data acquired during the same treatment session. The imaging data acquired during the treatment session may be used to calculate a radiation fluence that, when emitted by the radiotherapy system, delivers a conformal radiation dose to the tumor(s) that is less susceptible to artifacts and noise in the imaging data. In one variation, the location of the tumor(s) may be estimated during the treatment session using two or more algorithms, and then the two or more estimated locations may be combined to generate a final location that is used to calculate the radiation fluence for delivery. One or more of the algorithms may be statistical methods that may be used to analyze the real-time acquired imaging data. This approach may be helpful in situations where the acquired imaging data is sparse and/or noisy. This may help reduce or eliminate the impact of imaging artifacts on the delivery radiation fluence map (i.e., the fluence map intended for delivery to the target region), which may improve the conformality and/or quality of the delivered dose, thereby depositing more radiation on the target region and less radiation on healthy tissue.

Dose conformality is a measure of how well a radiation dose distribution covers the shape, size, and/or volume of the target region. A radiation dose with high conformality adheres closely to the shape, size, and/or volume of the target region, depositing a desired amount of radiation to the contours of the target region and limits “spill over” dose into non-target tissue, and/or missing dose to (e.g., underdosing) portions of the target region. A radiation dose with low conformality may not follow the shape or volume of the target region well, which may result in higher levels of radiation that “spill over” to surrounding non-target tissue, underdosing portions of the target region, and/or overdosing portions of the target region (creating so-called “hot spots” of dose). A treatment plan or delivered radiation may deposit the desired dose to the entirety of the target region(s), but if significant or elevated levels of dose are deposited outside of the target region(s), such a treatment plan or radiation delivery would be deemed as less conformal than a dose distribution that deposits the desired dose to the target region(s) and limits the irradiation of non-target regions. There are several metrics for quantifying dose conformality, including, but not limited to a conformity index (CI), an inverse conformity index (nCI), a dose gradient index (GI), etc. The CI may be calculated as the ratio of the prescription dose isodose volume to the volume of the target region. The nCI may be calculated as the ratio of the prescription dose isodose volume times the volume of the target region to the volume of the target region that receives at least the prescription dose squared. A smaller nCI value indicates a more conformal dose, and the ideal value (i.e., perfect conformality) would be 1. The GI may be calculated as the ratio of the volume of half the prescription isodose to the volume of the prescription isodose. One or more dose conformality (e.g., conformity) metrics or indices may be evaluated for a treatment plan by simulating the delivery of radiation according to the plan, and calculating the amount of radiation delivered to the target region(s) and non-target regions. One or more dose conformality metrics or indices may be evaluated for a delivered radiation dose by calculating the amount of radiation emitted to the target region(s) and non-target regions during a radiation delivery session, and calculating the deposited dose distribution using imaging data (e.g., CT imaging data) of the target region(s) and non-target regions.

Other methods described herein may include generating a treatment plan based on an image of a tumor location, instead of generating a treatment plan directly based solely on the tumor image. For example, an image of a tumor location may comprise a Gaussian or delta function centered over the location of the tumor convolved with a position uncertainty histogram (e.g., an error bar) that may represent the variation of a tumor location estimation algorithm or method. The image of a tumor or target region location may be referred to as an “anchor point” or “target region anchor”, and the dose delivered by a treatment plan may be “locked” onto the anchor point defined during treatment planning, and then identified during treatment delivery. In some variations, a treatment plan may be generated using a location uncertainty cloud that is centered on the most likely target region location (e.g., anchor point or target region anchor). An uncertainty cloud may be a bounded region in a parameter space that represents the possible values of a system state or variable (e.g., target region location), given measurement noise, modeling error, or incomplete information. Each point within the cloud corresponds to a feasible state estimate, and the geometry or extent of the cloud indicates the magnitude and distribution of uncertainty. For example, a target location uncertainty cloud may comprise a plurality of points representing possible tumor locations. The density of the points in the cloud may be proportional to the likelihood that the tumor location is at those locations. The shape and size of the uncertainty cloud may be calculated from the variability (e.g., uncertainty, imprecision, error rate, etc.) of the location estimation methods or algorithms and/or a location uncertainty model that indicates the variability of an estimation of a target location in different conditions. In some variations, the shape and size of an uncertainty cloud may also be calculated at least in part from the latency in time (actual or estimated latency) between the acquisition of imaging data and the delivery of radiation in response to the acquired imaging data and/or any other known systemic biases or offsets. A treatment plan may comprise one or more of firing filters, planned fluence map(s), planned dose distribution(s), and/or radiotherapy system instructions that deliver the planned fluence map(s) and/or planned dose distribution(s). For example, firing filters (i.e., mapping operators or functions that convert image data into radiation fluence) may be generated during treatment planning based on an image of a tumor location and not directly based on an image of the tumor. Alternatively, or additionally, generating a treatment plan may comprise calculating a planned radiation fluence map and/or firing filters based on an image of the tumor where one or more artifacts have been removed or suppressed. For example, a treatment plan may be generated using an image of the tumor where motion artifacts, such as motion blur, have been suppressed or eliminated. Optionally, the location data of the tumor may be used in conjunction with the reduced-motion (or motion-frozen) image to generate one or more components of a treatment plan. This may help reduce or eliminate the impact of imaging artifacts on the treatment plan, e.g., the planned radiation fluence map, planned firing filters, and/or planned radiation dose. Reduced-motion images of a tumor may be used in conjunction with tumor location data to train one or more of templates, correlation filters, and/or motion models, and/or may also be used to test or estimate the accuracy of location estimation/estimator algorithms before a treatment delivery session. In some variations, a treatment plan and its corresponding firing filters may be generated using a location uncertainty cloud that is centered on the target region anchor.

### Radiotherapy Systems

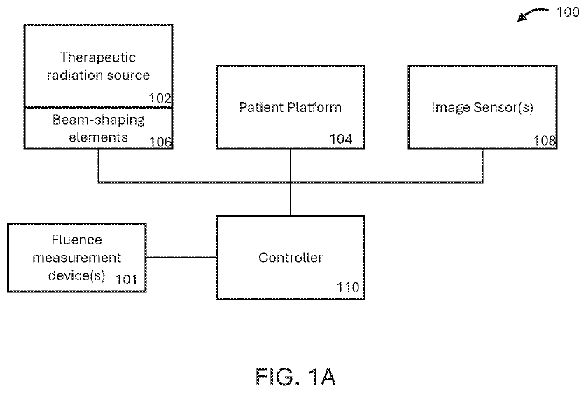

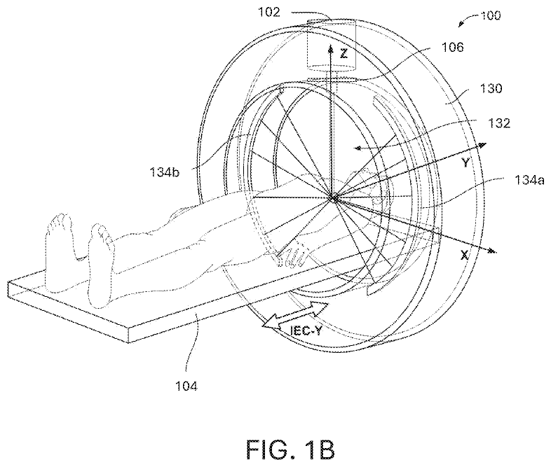

The methods described herein may be used with various radiotherapy systems, for example, any radiotherapy system comprising a therapeutic radiation source configured to emit high-energy photons and a radiation detector (also referred to as a radiation imager) located opposite the therapeutic radiation source. FIG. 1A depicts a functional block diagram of a variation of a radiotherapy system that may be used with one or more of the methods described herein. Radiotherapy system 100 comprises one or more therapeutic radiation sources 102 and a patient platform 104. The therapeutic radiation source may comprise an X-ray source, electron source, proton source, a neutron source, and/or any suitable particles including carbon atoms. For example, a therapeutic radiation source 102 may comprise a linear accelerator (linac), Cobalt-60 source, and/or an X-ray machine. The therapeutic radiation source may be movable about the patient platform so that radiation beams may be directed to a patient on the patient platform from multiple firing positions and/or angles. In some variations, a radiotherapy system may comprise one or more beam-shaping elements and/or assemblies 106 that may be located in the beam path of the therapeutic radiation source. For example, a radiotherapy system may comprise a linac 102 and a beam-shaping assembly 106 disposed in the radiation beam path of the linac. The beam-shaping assembly may comprise one or more movable jaws and one or more collimators. At least one of the collimators may be a multi-leaf collimator (e.g., a binary multi-leaf collimator, a 2-D multi-leaf collimator, etc.), which may comprise a plurality of independently movable leaves. The linac and the beam-shaping assembly may be mounted on a gantry or movable support frame that comprises a motion system configured to adjust the position of the linac and the beam-shaping assembly. In some variations, the linac and beam-shaping assembly may be mounted on a support structure comprising one or more robotic arms, C-arms, gimbals, and the like.

The patient platform 104 may be movable. For example, the patient platform 104 may be configured to translate a patient linearly along a single axis of motion (e.g., along the IEC-Y axis), and/or may be configured to move the patient along multiple axes of motion (e.g., 2 or more degrees of freedom, 3 or more degrees of freedom, 4 or more degrees of freedom, 5 or more degrees of freedom, etc.). In some variations, a radiotherapy system may have a 5-DOF patient platform or a 6-DOF patient platform that is configured to move along the IEC-Y axis, the IEC-X axis, the IEC-Z axis, as well as pitch, yaw, and/or roll.

In the variation shown in FIG. 1A, the radiotherapy system 100 also comprises a controller 110 that is in communication with the therapeutic radiation source 102, beam-shaping elements or assemblies 106, patient platform 104, and one or more image sensors 108 (e.g., one or more imaging systems). The controller 110 may comprise one or more processors and one or more machine-readable memories in communication with the one or more processors, which may be configured to execute or perform any of the methods described herein. The one or more machine-readable memories may store instructions to cause the processor to execute modules, processes and/or functions associated with the system, such as one or more treatment plans, the calculation of radiation fluence maps based on treatment plan and/or clinical goals, location estimation methods or algorithms (e.g., such as those described herein), deblurring methods, dosimetry calculations based on a beam model, acquisition of measurement data and/or analysis of imaging data, segmentation of fluence maps into radiotherapy system instructions (e.g., that may direct the operation of the gantry, therapeutic radiation source, beam-shaping assembly, patient platform, and/or any other components of a radiotherapy system), and image and/or data processing associated with treatment planning and/or radiation delivery. In some variations, the memory may store treatment plan data (e.g., treatment plan firing filters which may be transform functions that convert imaging data into fluence maps, fluence maps, beamlet sequence, planning images, treatment session PET prescan images and/or initial CT, MRI, and/or X-ray images), and instructions for delivering the derived fluence map (e.g., instructions for operating the therapeutic radiation source, beam-shaping assembly and patient platform in concert). In some variations, the controller 110 may comprise a processor for each of the therapeutic radiation source, beam-shaping elements, patient platform, and/or image sensors, with corresponding machine-readable memories. For example, in one variation, there may be an MLC processor and corresponding machine readable memory that generates and/or stores MLC instructions that specify the position, motion, location, timing (e.g., transition or travel time, dwell time, etc.) of each of the leaves of the MLC. The MLC instructions may be generated by a treatment planning system and stored in the memory associated with the MLC processor. Alternatively, or additionally, the MLC instructions may be generated by the MLC processor (e.g., in real-time) by the MLC processor, based on updated fluence maps and/or fluence maps generated based on imaging data (e.g., biology-guided radiotherapy which generates fluence maps from newly-acquired biologically-related imaging data, such as PET data). The controller of a radiotherapy system may be connected to other systems by wired or wireless communication channels. For example, the radiotherapy system controller may be in wired or wireless communication with a radiotherapy treatment planning system controller such that fluence maps, firing filters, initial and/or planning images (e.g., CT images including cone beam CT images and fan beam CT images, MRI images, PET images, 4-D CT images), imaging data, patient data, and other clinically-relevant information may be transferred from the radiotherapy treatment planning system to the radiotherapy system. The delivered radiation fluence, any dose calculations, and any clinically-relevant information and/or data acquired during the treatment session may be transferred from the radiotherapy system to the radiotherapy treatment planning system. This information may be used by the radiotherapy treatment planning system for adapting the treatment plan and/or adjusting delivery of radiation for a successive treatment session.

FIG. 1B depicts one variation of a radiotherapy system 100. Radiotherapy system 100 may include a gantry 130 rotatable about a patient treatment region 132, one or more PET detectors 134a, 134b mounted on the gantry, a therapeutic radiation source 102 mounted on the gantry, a beam-shaping module 106 disposed in the beam path of the therapeutic radiation source (and also mounted on the gantry), and a patient platform 104 movable within the patient treatment region 132. In some variations, the gantry 130 may be a continuously-rotatable gantry (e.g., able to rotate through 360° and/or in arcs with an angular spread of less than about 360°). The gantry 130 may be configured to rotate from about 20 RPM to about 70 RPM about the patient treatment region 132. For example, the gantry 130 may be configured to rotate at about 60 RPM. The gantry may also be configured to rotate at a slower rate, e.g., 20 RPM or less, 10 RPM or less, 1 RPM or less. The beam-shaping module 106 may include a movable jaw and/or a dynamic multi-leaf collimator (MLC) having one or more independently movable leaves. The beam-shaping module may be arranged to provide variable collimation width in the longitudinal direction of 1 cm, 2 cm, or 3 cm at the system iso-center (e.g., the rotation center of the system and on the central treatment or imaging plane, the center of a patient treatment area). The jaw may be located between the therapeutic radiation source and the MLC or may be located below the MLC. Alternatively, the beam-shaping module may include a split jaw where a first portion of the jaw is located between the therapeutic radiation source and the MLC, and a second portion of the jaw is located below the MLC and coupled to the first portion of the jaw such that both portions move together. The therapeutic radiation source 102 may be configured to emit radiation at predetermined firing positions (e.g., firing angles 0°/360° to 359°) about the patient treatment region 132. For example, in a system with a continuously-rotatable gantry, there may be from about 50 to about 100 firing positions (e.g., 50 firing positions, 60 firing positions, 80 firing positions, 90 firing positions, 100 firing positions, etc.) at various angular positions (e.g., firing angles) along a circle circumscribed by the therapeutic radiation source as it rotates. The firing positions may be evenly distributed such that the angular displacement between each firing position is the same.

The relative position between the linac and the beam-shaping assembly may be fixed such that the beam-shaping assembly remains in the beam path of the linac and the linac and beam-shaping assembly move together in concert. In some variations, although the relative position of the beam-shaping assembly and the linac may be fixed, the orientation of the beam-shaping assembly may be variable. For example, the beam-shaping assembly may be affixed to the gantry such that it is in the same motion frame as the linac, but the beam-shaping assembly may be tilted, angled, rotated, and/or shifted (i.e., central axis may be offset) relative to the linac. This variability may not be intentional (e.g., a result of manufacturing tolerances and/or imprecisions and/or motion) for some radiotherapy systems, and may be intentional for other radiotherapy systems. Some radiotherapy systems may have beam-shaping components whose positions and/or orientations are controlled by actuators and/or various mechanical adjustment mechanisms so that their orientations and/or positions may be adjustable relative to the linac. In most of the examples described herein, the radiotherapy system is one in which the position and orientation of at least one component of the beam-shaping assembly is intended to be fixed relative to the linac, and variabilities in position or orientation are unintended effects resulting from assembly, manufacture, and/or motion (e.g., rotational motion of the therapeutic radiation source).

### Methods

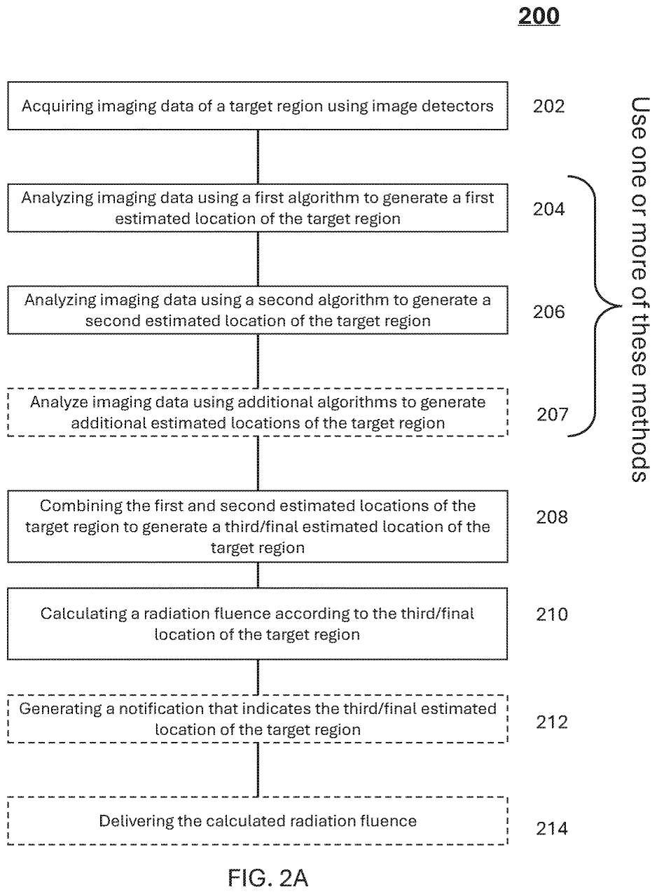

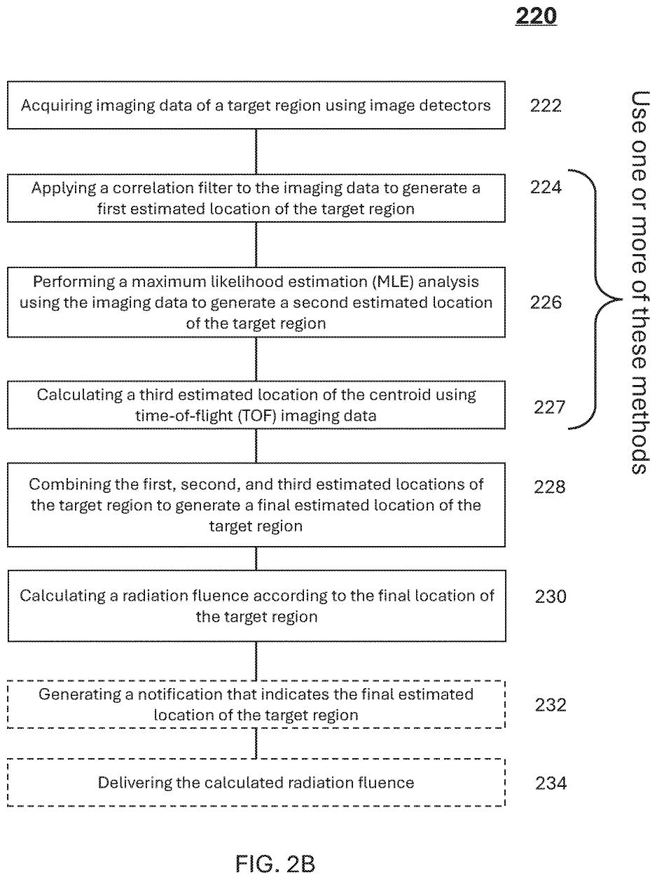

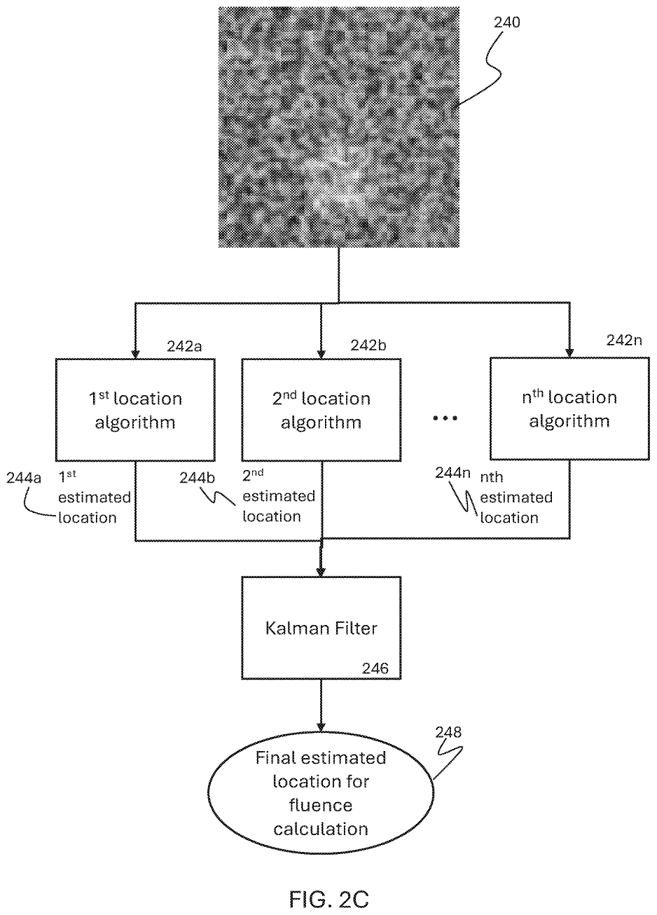

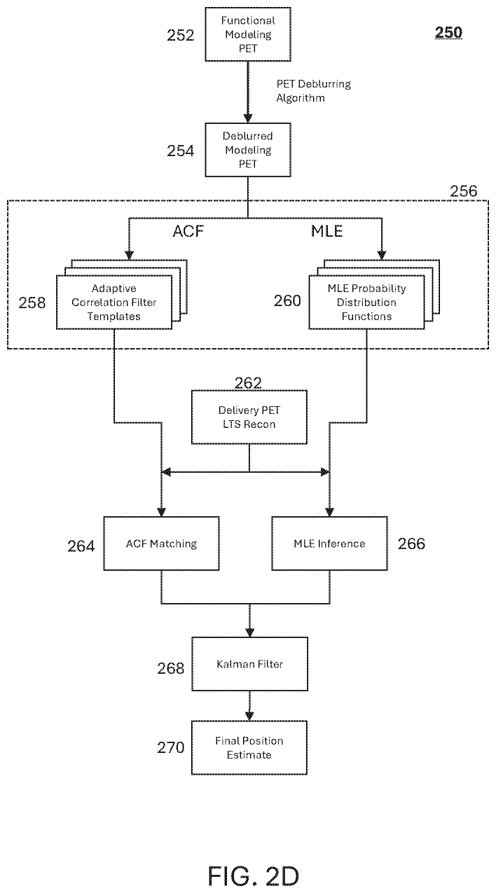

FIG. 2A depicts a flowchart representation of one variation of a method for generating an estimated location of a target region using imaging data acquired during the radiation delivery session and delivering radiation to the location of the target region, where artifacts in the imaging data have little or no impact on the conformality of the delivered radiation dose. Method 200 may comprise acquiring 202 imaging data of a target region using image detectors, analyzing 204 the imaging data using a first algorithm to generate a first estimated location of the target region, analyzing 206 the imaging data using a second algorithm to generate a second estimated location of the target region, combining 208 the first and second estimated locations of the target region to generate a third/final estimated location of the target region, and calculating 210 a radiation fluence according to the third/final location of the target region. The location of the target region may include 3-D (e.g., x, y, z) coordinates of the target region centroid or selected landmark relative to coordinate axis of the treatment room or radiotherapy system. Optionally, some methods may comprise applying “position memory” to the final estimated location of the target region. This may include, for example, using the recent history of target locations to prevent the current position estimate from jumping erratically. Applying “position memory” may rejects target position estimates that are not physiologically plausible. In some variations, the method may comprise applying “confidence rejection” to rejects position estimates with confidence values below a threshold determined during treatment planning. This may include, for example, calculating an uncertainty model based on the acquired 202 imaging data and evaluating the characteristics of the uncertainty model. For example, if the uncertainty model has a spread (e.g., where spread is 2 standard deviations) that exceeds a threshold (e.g., 20 mm or more) and/or a mean value (e.g., a shift amount) that exceeds a threshold (e.g., 3 mm or more), the estimated final location 208 may be considered unreliable. This may help reject position estimates with too much uncertainty. Method 200 may comprise generating a GUI that includes a notification indicating that the final location 210 may not be reliable and prompt the user to take alternative action (e.g., pause delivery, re-acquire imaging data, etc.). In some variations, method 200 may comprise analyzing 207 the imaging data using a third algorithm to generate a third estimated location of the target region, which may be combined 208 with the other estimated locations to generate a final estimated location of the target region. The imaging data may be PET imaging data (e.g., lines-of-response LOR data, positron annihilation emission path data, and/or listmode data which may comprise positron annihilation event data, timing of detection of coincident photons, and/or energy of detected photons, etc.), CT imaging data (e.g., projection data, X-ray data), MR imaging data, ultrasound imaging data, and the like. The imaging data may be acquired 202 using an imaging system that is on-board with the radiotherapy system, which may facilitate the rapid delivery of radiation to the target region before the imaging data and location data of the target region become “stale” (i.e., the imaging and location data no longer indicate the actual, real-time properties of the target region). In one variation, PET imaging data may be acquired 202 using PET detectors mounted on a rotatable gantry, similar to the systems described above.

Calculating 210 a radiation fluence using the third/final location of the target region may comprise generating an image of the location of the target region, and applying (e.g., convolving) the image of the target region location with firing filters calculated during treatment planning. The image of the target region location may also be referred to as an anchor point image. In some variations, an image of the target region location may comprise a Gaussian, delta function, centered over the third/final location of the target region. For example, an anchor point image may comprise a 3D Gaussian having a width of 1 pixel centered over the estimated location of the target region. During treatment planning, the firing filters may be calculated using an image of the target region location (e.g., a Gaussian, delta function located at the target location and/or anchor point) and/or a location uncertainty model (e.g., a location uncertainty cloud) associated with the location estimation methods for the target location and/or anchor point, and the parameters of the image of the target region location may be used during treatment delivery to generate the image of the target region location. For example, if the firing filters were generated during treatment planning using a Gaussian function have a certain mean value and variance value, a Gaussian function with the same mean value and variance value may be used during treatment delivery to generate an image of the target region location. The firing filters may be a mapping operator, conversion function, and/or transformation function that converts imaging data into radiation fluence. In the examples described herein, the image of the target region location (i.e., anchor point image) may be converted to a radiation fluence for delivery by the firing filters. A firing filter may be derived for each firing position of the therapeutic radiation source and during a radiation delivery session, applied a projection of the imaging data onto the respective firing position. In some variations, calculating a fluence for delivery may comprise projecting the anchor point image into beam's eye view projections, and convolving these projections with the firing filters from the treatment plan. The set of firing filters from the treatment plan may comprise a set of per-firing-angle (e.g., beam's eye view) firing filters. The fluence for each firing position may be computed based on the anchor point projections derived from the LTS PET image reconstructed using the PET data in the most recent LTS PET time window period (i.e., 500 ms or 4 sec, depending on the selected tracking mode). Alternatively, or additionally, calculating 210 a radiation fluence using the third/final location of the target region may comprise shifting a planned radiation fluence map to the third/final location of the target region. In some variations shifting the planned fluence map may comprise aligning the portion of the planned fluence map corresponding to the location of the target region to the final estimated location of the target region calculated in step 208.

Method 200 may optionally comprise generating 212 a notification that indicates the third/final estimated location. The notification may comprise a graphical user interface (GUI) that includes a visual depiction of the estimated target region location relative to other landmarks or indicia of interest. For example, the visual depiction may include one or more of the target region contour(s), patient anatomical structures (e.g., specific bones and other rigid landmarks), previous location estimate(s) for the target region, contour(s) and/or location(s) of organs at risk (OARs, and/or any anatomical structures for which radiation exposure should be limited). In some variations, the notification may comprise a visual and/or audible alert indicating whether the estimated location of the target region is within an acceptable range of locations, or whether the estimated location is outside the acceptable range of locations. Method 200 may further comprise delivering 214 the radiation fluence that has been calculated/generated based on the third/final location of the target region. The radiation may be delivered using the therapeutic radiation source of the radiotherapy system.

While method 200 has been described as using multiple algorithms to generate estimates of the target region location, it should be understood that in other variations, a single algorithm may be used to generate an estimate of the target region location. The single location estimate may be used to calculate the radiation fluence for delivery. In such a situation, there may be a sufficiently high level of confidence or certainty that the single location estimation/estimator algorithm when applied to the acquired imaging data will result in an accurate estimate of the target region location. For example, the acquired imaging data may be of sufficient quality for a single location algorithm to estimate the location of the target region with a high degree of confidence. If a plurality of algorithms is used to estimate the location of the target region, these algorithms may be executed simultaneously, sequentially, or over different (e.g., separate, overlapping) time intervals. For example, a first algorithm may be used to generate a location estimate every 500 ms, while a second algorithm may be used to generate a location estimate every few seconds (e.g., 2-5 seconds), and combining these location estimates may comprise corroborating the location estimates from both algorithms to identify whether there is any bias in one of the algorithms, and compensating for that bias using the other of the algorithms. For example, a first algorithm for estimating a target region location may provide an instantaneous location estimate, while a second algorithm may maintain a running average of the location estimates. If the instantaneous location estimate indicates a sudden change or shift from the running average of the location estimates, a signal may be generated to the radiotherapy machine to pause the delivery of radiation until the target region position (e.g., the position of the patient) may be ascertained. This may be performed by a third algorithm, and/or a user verifying that the patient is in place, and/or acquiring additional imaging data of the patient and/or target region.

In some variations, the location estimation/estimator algorithms used in method 200 may also be used to generate estimated locations of one or more organs-at-risk or OARs. The steps 202, 204, 206, 207 (optional) and 208 may be used to estimate the location of an OAR. The estimated location of the one or more OARs may be used to confirm, check, and/or help further refine the estimated location of the target region. In some variations, tracking the location of the one or more OARs may improve the estimation of the target region location. Calculating 210 the radiation fluence for delivery may consider or account for the location(s) of the one or more OARs so that the radiation fluence may be delivered to the target region with little or no irradiation of the OAR(s). For example, the radiation fluence may be specifically calculated to avoid irradiating the OAR(s) while still delivering the prescribed radiation dose to the target region. Optionally, a GUI having a visual depiction (e.g., plot or diagram) of the updated estimate of the target region location may also include an updated estimate of the OAR location, so that the target region and the OAR(s) may be tracked together. The visual depiction may help a therapist or clinician determine whether to continue with radiotherapy treatment or pause treatment to re-position the patient or re-calculate the radiation fluence.

FIG. 2B depicts a flowchart representation of one variation of the method 200 depicted and described in FIG. 2A. FIG. 2C is a conceptual diagram depicting how acquired imaging data is analyzed using the methods of FIGS. 2A-2B to generate an estimated location of the target region. The estimated location of the target region (e.g., an anchor point image), instead of the acquired imaging data itself, may be used to calculate the radiation fluence that is delivered to the patient. This may help limit or eliminate the impact of artifacts of the acquired imaging data on the radiation dose that is delivered to the target region. Method 220 may comprise acquiring 222 imaging data of a target region using image detectors, applying 224 a correlation filter to the imaging data to generate a first estimated location of the target region, performing 226 a maximum likelihood estimation (MLE) analysis using the imaging data to generate a second estimated location of the target region, calculating 227 a third estimated location of the target region location using time-of-flight (TOF) imaging data, combining 228 the first, second, and third estimated locations of the target region to generate a final estimated location of the target region, and calculating 230 a radiation fluence according to the final location of the target region. The methods may comprise generating an anchor point image comprising a delta function or a Gaussian function placed over the location of the target region. For example, an anchor point image may comprise a 3D Gaussian having a width of 1 pixel centered over the estimated location of the target region. In some variations, fewer than three location estimate algorithms may be used. For example, one variation may comprise a first algorithm using a correlation filter and a second algorithm using maximum likelihood estimation analysis. Another variation may comprise a first algorithm using a correlation filter and a second algorithm using time-of-flight PET imaging data to locate a centroid of the target region. Yet another variation may comprise a first algorithm using maximum likelihood estimation analysis and a second algorithm using time-of-flight PET imaging data. Optionally, method 220 may comprise generating 232 a notification that indicates the final estimated location of the target region, and/or delivering 234 the calculated radiation fluence, as depicted and described above for FIG. 2A. Optionally, some methods may comprise applying “position memory” to the final estimated location of the target region. This may include, for example, using the recent history of target locations to prevent the current position estimate from jumping erratically. Applying “position memory” may rejects target position estimates that are not physiologically plausible. In some variations, the method may comprise applying “confidence rejection” to rejects position estimates with confidence values below a threshold determined during treatment planning. This may include, for example, calculating an uncertainty model based on the acquired 202 imaging data and evaluating the characteristics of the uncertainty model. For example, if the uncertainty model has a spread (e.g., where spread is 2 standard deviations) that exceeds a threshold (e.g., 20 mm or more) and/or a mean value (e.g., a shift amount) that exceeds a threshold (e.g., 3 mm or more), the estimated final location 208 may be considered unreliable. This may help reject position estimates with too much uncertainty. In some variations, calculating a fluence for delivery may comprise projecting the anchor point image into beam's eye view projections, and convolving these projections with the firing filters from the treatment plan. The set of firing filters from the treatment plan may comprise a set of per-firing-angle (e.g., beam's eye view) firing filters. The fluence for each firing position may be computed based on the anchor point projections derived from the LTS PET image reconstructed using the PET data in the most recent LTS PET time window period (i.e., 500 ms or 4 sec, depending on the selected tracking mode). Method 200 may comprise generating a GUI that includes a notification indicating that the final location 210 may not be reliable and prompt the user to take alternative action (e.g., pause delivery, re-acquire imaging data, etc.). In some variations, the estimated location may be the location of the target region centroid, but the estimated location may be any tumor landmark and/or feature and/or anchor that may be used to represent the target region location, and may be coordinates, e.g., in 3 dimensions (x,y,z), and/or a 3 dimensional shift vector relative to a predetermined position. The imaging data may be PET imaging data, and the image detectors may be PET detectors. The correlation filter may be, in some variations, an adaptive correlation filter, as described further below and also described in PCT Application No. PCT/US2023/079652 filed Nov. 14, 2023, which is hereby incorporated by reference in its entirety. Maximum likelihood estimation analysis and time-of-flight imaging data analysis for generating an estimated location of a target region are described further below.

FIG. 2C conceptually depicts the steps of methods 200 and 230 from FIGS. 2A and 2B. An example PET image 240 may be acquired by the PET detectors on a radiotherapy system. In some variations, for each patient platform beam station, there may be a plurality of PET images acquired that are similar to image 240. The methods 200, 220 may then comprise analyzing the image(s) 240 using one or more location algorithms 242a, 242b, . . . 242n. These location algorithms may include, but are not limited to, correlation filters, template matching, statistical methods such as maximum likelihood estimation, PET time-of-flight data analysis, and the like. The estimated locations 244a, 244b, . . . 244n from each of these algorithms may be combined, for example, using a Kalman filter 246 to attain a final estimated location 248 for radiation fluence calculations. While a Kalman filter is used in this example to combine the location estimates of the target region from multiple location algorithms, in other variations, the location estimates may be combined by finding the center-of-mass of the plurality of location estimates.

FIG. 2D depicts one variation of a radiotherapy treatment planning and delivery method for determining real-time estimates of a target location during radiation delivery. Method 250 may comprise acquiring 252 a planning PET image (which may be referred to as functional modeling PET image), applying 254 a deblurring algorithm to the planning PET images to obtain a deblurred planning PET image (referred to as a deblurred modeling PET image), and training 256 one or more location estimation methods with the deblurred planning PET image. Deblurring algorithms may correct, reduce and/or remove motion artifacts from the PET images that may cause the target region to appear “blurred”. “Training” a location estimator method may also be referred to as “initializing” a location estimator method, where training or initializing a location estimator method comprises generating and/or calculating the parameters, templates, models, and/or filters of the location estimator method. While some examples described herein use “train” or “training”, it should be understood that those terms may be interchangeable with “initialize” or “initializing”, and/or “update” or “updating” (in variations where the elements of a location estimator method are generated or calculated again, usually based on newly acquired imaging data). The motion-corrected (i.e., “deblurred”) images may better approximate the location of the target region in the planning PET images as compared to the “blurred” images. In some variations, the deblurred images may contain clearer and/or more precise images of the target region than “blurred” images. The location estimation methods described herein may be trained on the motion-corrected images. For example, method 250 may comprise calculating 258 one or more adaptive correlation filters (ACFs; also referred to as templates) based on the deblurred planning PET images and/or generating 260 a plurality of probability density functions for a plurality of shifts using the deblurred planning PET images. The calculated ACFs and generated probability density functions may be used in the location estimation methods that are described further below. In some variations, the location estimation quality of the location estimation algorithms may be evaluated and tested on PET imaging data. The estimated locations of the target region may be compared with the locations of the target region as determined by the deblurring algorithm. The location differences may be used to create a location uncertainty model or uncertainty cloud. The radiotherapy treatment plan may be generated based on the location uncertainty model or uncertainty cloud centered on the location of the target region. The trained location estimation methods and associated templates and/or probability density functions (and/or any other associated location estimation data) and/or uncertainty model (e.g., uncertainty cloud), firing filters may be saved in one or more machine-readable memories of a controller and/or processor, which may then be accessed during a radiation delivery session. During a radiation delivery session when radiation is delivered to a PET-avid target region (of either a phantom during a quality assurance session, or of a patient during a radiotherapy session), the radiotherapy system may access the one or more memories for the trained location estimation methods and associated location estimation data. The location estimation methods may then be applied to PET imaging data acquired during the radiation delivery session (e.g., in real-time) to estimate the location of the target region. In one variation, method 250 may comprise acquiring 262 PET imaging data from the PET-avid target region (which may be referred to as reconstructed limited-time sampled (LTS) PET images acquired during delivery, “Delivery PET LTS recon”), applying 264 a correlation filter to the PET imaging data to generate a first estimated location of the target region, and/or performing 266 a maximum likelihood estimation (MLE) analysis using the imaging data to generate a second estimated location of the target region, combining 268 the first and second estimated locations of the target region to generate a final estimated location 270 of the target region. While the location estimation methods used in method 250 comprise applying a correlation filter and/or performing a MLE analysis, other variations may use one or more other location estimation methods instead of, or in addition to, these methods. Some methods may comprise generating an image of an anchor function based on the final estimated location that may be a delta function or Gaussian function (or any desired distribution) centered at the final estimated location, and calculating the radiation fluence for delivery by applying a firing filter to the image of the anchor function.

Method 250 may also comprise acquiring PET imaging data before radiation delivery commences. This pre-radiation PET imaging acquisition may be referred to as a PET prescan. The PET imaging data from the PET prescan may be used to test/evaluate the trained location estimation methods to determine whether they are able to reliably estimate the location of the target region. In one variation, method 250 may comprise deblurring the PET imaging data from the PET prescan to generate a dataset comprising the PET prescan imaging data (e.g., PET LTS images derived from PET LOR data) binned into motion phases. The PET prescan imaging data may comprise a series of limited-time sampled (LTS) images reconstructed from PET LOR data, and dataset resulting from deblurring the PET prescan imaging data may comprise a plurality of motion phases or bins, LTS images acquired during that motion phase, and location information about the target region. The location information may include target region shift information, such as the magnitude and/or direction of the shift from the previously-known or reference location, in 2D or 3D vectors. The location information may optionally include 2D or 3D coordinate values of the target region location. The dataset from deblurring the PET prescan imaging data may be used to generate location estimation quality metrics of the location estimation methods that have been trained for the radiation delivery session. These location estimation quality metrics may be, for example, a location uncertainty model and/or tracking error metric. These metrics may quantify the amount of confidence that a user may have in the accuracy of the location estimation methods. Additional details of generating an uncertainty model from acquired PET imaging data for location estimation methods are described further below. The uncertainty model generated from the PET prescan data may be compared with the uncertainty model generated during treatment planning. For example, the spread/size of the uncertainty models from the PET prescan and from treatment planning may be compared. Optionally, the average value of the uncertainty models from the PET prescan and from treatment planning may be compared. If the characteristics of the uncertainty models differ or vary beyond an acceptable threshold, a notification may be generated and output to the user to indicate this deviation outside the acceptable range and prompt the user for additional reviews and/or confirmation of whether the user wishes to proceed with radiation delivery. For example, if the location uncertainty model at the time of treatment has a spread and/or average value that is unacceptably different from the spread and/or average value of the location uncertainty model at treatment planning, the user may decide to retrain and/or update the location estimation methods using the PET prescan imaging data, and then re-evaluate the location uncertainty model metrics of the retrained/updated location estimation methods. If the characteristics of the location uncertainty model of the retrained location estimation methods are within an acceptable range, the radiotherapy system controller may generate a notification indicating that radiation delivery may proceed (or at least proceed to another safety evaluation). The evaluation of the reliability and/or confidence level of the location estimation methods may be an important safety check to confirm that the PET signal, anatomical and/or biological state of the patient on the day of treatment allows for sufficiently accurate estimations of the target region. In some variations, the evaluation of the location uncertainty model derived from the PET prescan imaging data may be used as a “GO” or “NO GO” criterion for proceeding with radiation delivery and/or the next stage of safety checks.

As used throughout limited time-sampled (LTS) images may refer to images that are generated based on imaging data acquired within shortened acquisition windows. In some variations, an imaging system (e.g., of a radiotherapy system or a diagnostic imaging system) may have a first mode where limited time-sampled (LTS) images are calculated from imaging data acquired over 500 ms and updated every 100 ms, and a second mode where LTS images are calculated from data acquired over 4 seconds and updated every 100 ms. The acquisition window or mode for LTS images acquired during a planning PET imaging session and/or during a PET-guided radiotherapy session may be selected by the user. For example, the acquisition mode for LTS images during planning and/or delivery may be set to the first mode if a user expects the target region to move at a frequency that approximates breathing, such as for lung tumors and/or tumors in the ribs and sternum. In another example, the acquisition mode for LTS images during planning and/or delivery may be set to the second mode if a user expects the target region to be subject to small sudden patient shifts, such as for bone tumors that are not near the ribs or sternum.

The PET imaging data from the PET prescan may also be used to calculate an estimate of the dose that may be delivered to the target region and/or patient. The PET prescan imaging data may be used to calculate a predicted delivered dose to the target region and/or patient. For example, the location estimation methods may be applied to the PET prescan imaging data to identify the location(s) of the target region anchor, and a delivery fluence may be calculated by applying the planned firing filter(s) to the location(s) of the target region anchor. The delivery fluence may be summed across all beam stations and firing positions to estimate a predicted cumulative delivered dose. For example, this predicted radiation dose may be represented as a dose volume histogram (DVH) for the target region and/or any organs at risk (OARs). The DVH calculated from PET prescan imaging data may be compared to a bounded DVH that includes a lower bound DVH (representing a minimum acceptable dose) and an upper bound DVH (representing a maximum acceptable dose) for the target region and/or organs at risk. If the predicted delivery dose calculated from the PET prescan for the target region and/or OARs is/are not within an acceptable dose range, the radiotherapy system controller may generate a notification (e.g., an audible notification output to a speaker, a graphical notification output to a display device, and the like) to the user indicating that they may wish to stop, pause, and/or adapt the delivery, and/or the predicted delivery dose calculated from the PET prescan is within an acceptable dose range. The evaluation of the predicted delivered dose may be an important safety check to confirm that the PET signal, anatomical and/or biological state of the patient on the day of treatment allows for delivery of the prescribed radiation dose to the target region while not over-irradiating OAR(s). In some variations, the evaluation of the predicted delivered dose derived from the PET prescan imaging data may be used as a “GO” or “NO GO” criterion for proceeding with radiation delivery and/or the next stage of safety checks.

### Maximum Likelihood Estimation (MLE) Location Estimation Methods

In some variations, one or more statistical algorithms or methods may be used to generate an estimated location of a target region using sparse and/or low-resolution imaging data of the target region. The estimated location may then be used to calculate a radiation fluence for delivering a conformal radiation dose to the target region, where the radiation dose may be less susceptible to noise from the imaging data. Statistical methods may be suitable for analyzing sparse, incomplete, and/or noisy imaging data (e.g., with low signal-to-noise ratios, low photon count, low contrast from background, blurry or poorly-defined edges, low resolution, etc.) where it may be difficult or impossible to visually identify the location of a target region centroid. Imaging data of such poor quality may be problematic for methods that rely on ample, complete, and/or high-quality imaging data (e.g., with high signal-to-noise ratios, high photon count, high contrast from background, well-defined edges, etc.) to accurately locate the target region. However, statistical methods may be well-suited to analyze incomplete and/or noisy imaging data to facilitate the identification of the target region location. Statistical methods, such as maximum likelihood estimation (MLE) methods may mitigate the problem of identifying a target region location within a poor quality image, e.g., low-photon count image, images with sparse, incomplete and/or noisy imaging data. Statistical methods are intended to analyze data of stochastic nature, and may therefore be especially appropriate for PET images, since PET images are generated based on the stochastic emissions of positrons by a PET tracer. While the examples and variations disclosed herein related to PET images and imaging data (e.g., lines-of-response LORs data, listmode data, positron annihilation emission path data, etc.), it should be understood that the methods may also be applied to other imaging modalities, including, but not limited to, fluoroscopy, X-ray imaging, MRI, CT, ultrasound, and the like.

One variation of an MLE method for determining the location of a target region from a poor quality image may comprise acquiring a low-photon count image of the target region that has sparse and/or incomplete imaging data and applying a plurality of probability density functions that each correspond to a different target region shift to the low-photon count image. A probability density function (PDF) is a function that assigns a likelihood to each possible value of a continuous random variable. The integral of the PDF over any region specifies the probability that the variable lies within that region, and the shape of the PDF characterizes the statistical distribution of uncertainty. In other words, a probability density function is a statistical expression that defines the probability that a particular condition will occur. For the methods disclosed herein, a probability density function for a particular target region location or shift is an expression that indicates the probability that the target region is at that location or has moved by that shift. In some variations, the probability density function for a particular target region location and/or shift is a distribution that is centered over the specific location and/or shift, and has a spread (i.e., a width) that indicates the uncertainty of detecting that specific location and/or shift. The method may comprise calculating a likelihood value for each target region shift, identifying the target region shift that has a likelihood value that exceeds a threshold (e.g., the largest or maximum likelihood value of all of the different target region shifts), and then calculating the (updated) location of the target region by applying the identified target region shift to an initial location (or last known location) of the target region. The likelihood value for each target region shift may be calculated from the acquired imaging data of the target region and applying a plurality of PDFs to the acquired imaging data. Once the location of the target region is determined using one or more of the methods described above and herein, the radiation fluence to be delivered may be calculated using the target region location (e.g., data or information about the location of the target region). Calculating the radiation fluence for delivery based on a target region location may provide improved dose conformality as compared to a radiation fluence calculated directly from the low-photon count image of the target region.

In some variations, a probability density function may be derived from one or more previously-acquired images of the target region at a single (e.g., initial, nominal) location, where the previously-acquired images may be higher-quality images (e.g., planning PET images, diagnostic PET images, a high-photon count PET image) relative to the poor quality image (e.g., PET images generated using data acquired in short time intervals (e.g., less than 10 seconds, less than 5 seconds, less than 1 second). These images may be acquired at any point before radiation delivery, for example, during treatment planning and/or radiation delivery modeling. In some variations, poor quality imaging data may comprise a population of lines-of-response (LORs or positron annihilation emission paths). A probability density function for a tumor or target location may include a map of the probability (e.g., likelihood) values that the tumor or target region is located at a plurality of possible locations or places. As a tumor or target region moves and/or changes locations, the probability density function will change to indicate those location changes. A plurality of probability density functions may be generated by shifting the target region in an image to a plurality of different locations, and generating a probability density function for each of the images of the different locations and/or shifts. These different probability density functions that correspond to different target region shifts and/or locations may be applied to the low-photon count image to determine the location of the target region within the low-photon count image. Probably density functions may be generated using any imaging data of the target region, including one or more of imaging data acquired during treatment planning, imaging data acquired during a quality assurance (QA) session, imaging data acquired on the day of a treatment session, imaging data acquired just before the emission of radiation from a radiotherapy system, and/or imaging data acquired during radiation delivery to the target region. Some variations may comprise generating a look up table or matrix of the plurality of probability density functions, and the look up table may be stored in machine readable memory of the controller and/or processor that is accessed for estimating the location of the target region using the MLE method. In some variations, the updated location of the target region may be used to generate an additional probability density function that may be included in the plurality of probability density functions, which may be applied to any newly acquired or future acquired imaging data to further update the location of the target region. For example, imaging data acquired at an earlier timepoint during a radiotherapy session may be used to generate a probability density function that may be applied to imaging data acquired at a later timepoint during the same radiotherapy session. Additionally, or alternatively, generating a probability density function may comprise applying a Bayesian model to a previously-calculated or previously-known location of the target region.

In some variations, applying the a probability density function to an image or imaging data may comprise cross-correlating the probability density function with the image or imaging data. Alternatively, or additionally, applying the a probability density function to an image or imaging data may comprise convolving the probability density function with the image or imaging data. The likelihood value calculated from applying a probability density function to imaging data may indicate the probability that the target region is at a location corresponding to the shifted location represented by the probability density function. A high likelihood value associated with a particular target region shift may indicate a high probability that the target region is located at the shifted location corresponding to that probability density function. These operations may be performed by a controller (e.g., processor) for a plurality of probability density functions corresponding to a plurality of target region shifts to obtain a plurality of likelihood values. The target region shift (or location) with the highest likelihood value and/or the likelihood that exceeds a pre-determined threshold may be used to estimate the location of the target region.

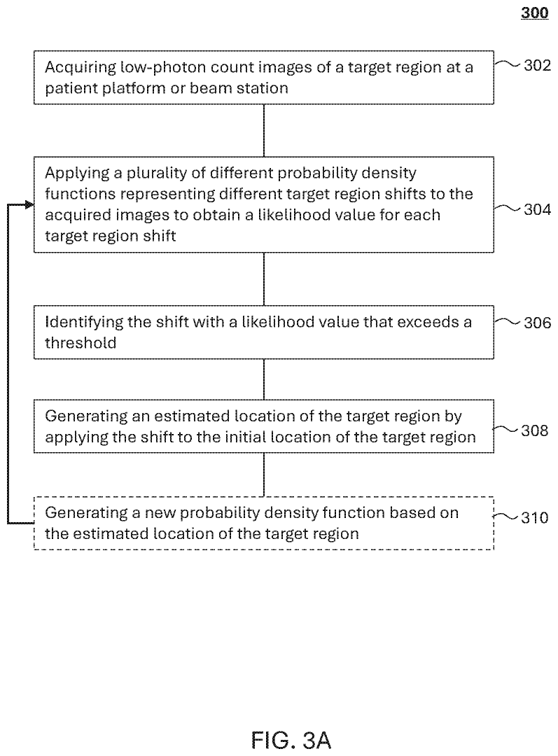

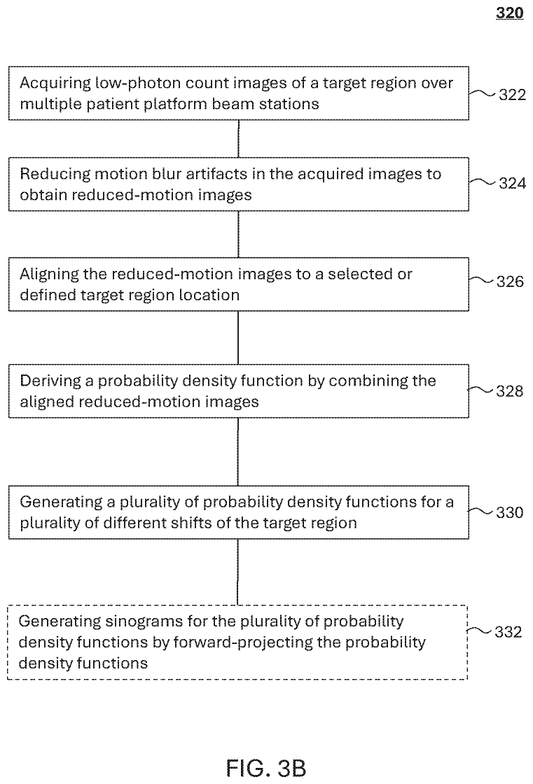

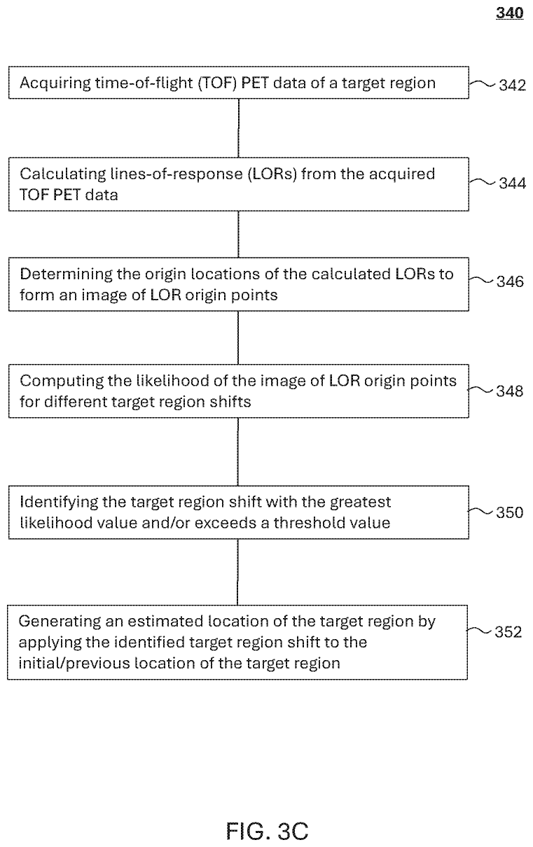

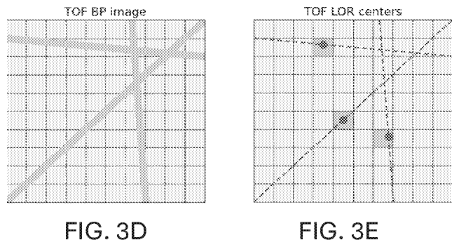

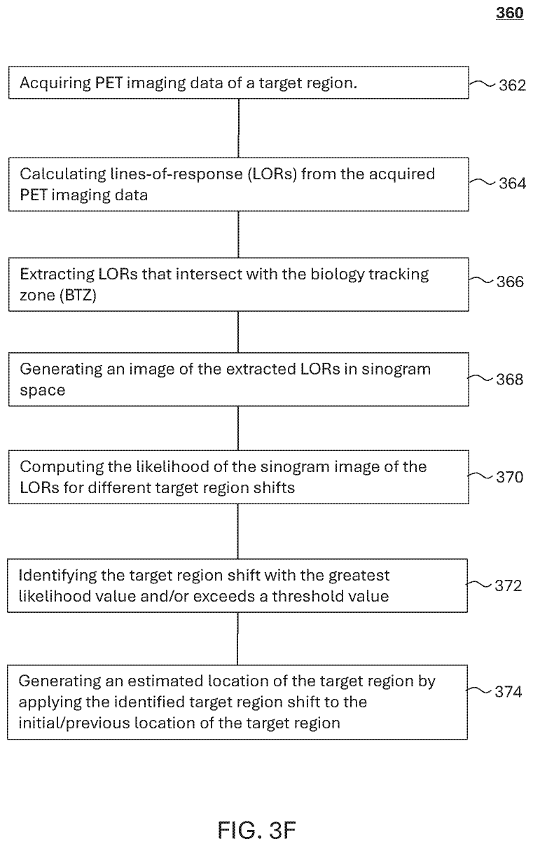

FIG. 3A is a flowchart representation of one variation of a statistical inference method that may be used to calculate a location of a target region from low-photon imaging data. The location of the target region, instead of the low-photon imaging data, may then be used to calculate the radiation fluence for delivery. Method 300 may comprise acquiring 302 low-photon count images or imaging data of a target region at a patient platform location (i.e., beam station), applying 304 a plurality of different probability density functions representing different target shifts to the acquired low-photon count images to obtain a likelihood value for each potential shift of the target region, identifying 306 the shift with a likelihood value that exceeds a threshold, and generating 308 an estimated location of the target region by applying the shift to an initial or previous location of the target. In some variations, low-photon images may be images that have been acquired within a short acquisition time window, such as limited time-sampled images acquired in 10 seconds or less, e.g., 5 seconds or less, 1 second or less, 0.5 seconds or less, etc. In some variations, an imaging system (e.g., of a radiotherapy system or a diagnostic imaging system) may have a first mode where limited time-sampled (LTS) are images are calculated from data acquired over 500 ms and updated every 100 ms, and a second mode where LTS images are calculated from data acquired over 4 seconds and updated every 100 ms. Applying 304 a probability density function to the acquired images may comprise cross-correlating the plurality of probability density functions with the acquired images or imaging data. Alternatively, or additionally, applying 304 a probability density function to the acquired images may comprise computing a dot product of the plurality of probability density functions with the acquired images or imaging data (e.g., sum of the product of the acquired images and the PDFs). In some variations, the plurality of different probability density function(s) may represent the likelihood of different target locations. Any of the methods described herein that use PDFs of different target region shifts may alternatively, or additionally, use PDFs of different target region locations.

In some variations, identifying 306 the shift with a likelihood value that exceeds a threshold, where the threshold represents a confidence level for a particular shift or location. For example, the likelihood threshold may be about 20% or more. That is, if the likelihood value for a probability density function is less than about 20%, the target region is unlikely to have moved by that shift value, and is therefore, unlikely to be present at that shifted location. If there are many shifts and probability density functions with likelihood values greater than about 20%, the identifying the shift may comprise identifying the shift with the highest likelihood value amongst all of the potential shifts with confidence levels above 20%. The estimated location of the target region may be coordinates, e.g., in 3 dimensions (x,y,z), and/or a 3 dimensional shift vector relative to a predetermined position. In some variations, the location of a target region may be the location of the centroid of the target region, and/or any tumor landmark and/or feature and/or anchor that may be used to represent the target region location. The target region centroid, landmark, feature and/or anchor may be selected or defined by a clinician or user during treatment planning. Generating 308 the estimated location of the target region may comprise by applying the shift to an initial or previous location of the location of a centroid of the target region.

Alternatively, or additionally, method 300 may be used with time-of-flight (TOF) PET imaging data. In one variation, method 300 may comprise acquiring TOF PET data of a target region, calculating LORs from the acquired TOF PET imaging data, generating an image (e.g., a back projection image) using the calculated LORs, and applying a plurality of different probability density functions representing different target region shifts to the generated back projection images to obtain a likelihood value for each target region shift. As described previously, this variation may comprise identifying the shift with a likelihood value that exceeds a threshold and generating an estimated location of the target region by applying the shift to the initial location of the target region.

Optionally, method 300 may comprise generating 310 a new probability density function based on the estimated location of the target region, which may be stored in a look up table with the pre-existing probability density functions. The new probability density function may be generated by multiplying the probability density function of the identified shift with the probability density function of previous shift or an adjacent shift. The newly generated or updated probability density function may be added to the set of different probability density functions and applied 304 to the next set of acquired images to generate the next estimate of the location of the target region. In some variations, images or imaging data may be acquired of the target region at the start of a treatment session before radiation is emitted. Such “prescan” images or imaging data may optionally be used to generate probability density functions for use in step 304 and/or may be used to identify the initial location of the target region. For example, a prescan image may be a patient localization image and/or any image acquired at the time a patient is localized to the radiotherapy system, and may have sufficient imaging data (e.g., high signal-to-noise ratio, high photon count, high contrast from background, well-defined edges, etc.) to identify the initial location of the target region.

Method 300 may be repeated throughout a radiation delivery or treatment session to continuously calculate the real-time location of a target region. In some variations, method 300 may be performed at least once for each patient platform position or beam station, where the patient platform is stopped during therapeutic beam delivery. Method 300 may be repeated after the patient platform is moved to another position or beam station, until radiation has been delivered at all planned beam stations. Alternatively, or additionally, method 300 may be performed continuously during radiation delivery, even while the patient platform is moving. In some variations where the patient platform is moved through the treatment beam multiple times during radiation delivery, where each crossing through the treatment beam is referred to as a “pass” and the repeated passes referred to as “shuttling” the patient platform or “shuttle passes”, one or more of the steps of method 300 may be performed at the end of a completed shuttle pass or prior to the beginning of the next shuttle pass. For example, generating 310 a new probability density function may use the estimated target location or shift from a previous pass (e.g., the just-completed pass) may occur between consecutive passes such that the next pass can use the newly generated probability density function to facilitate the generation of an updated estimated location of the target region.

The probability density functions used during a radiation delivery session may be generated before the radiation delivery session, for example, during treatment planning. The portion of the tumor to be used for estimating the tumor location may be defined or selected during treatment planning. In some variations, the portion of the tumor that is used to represent and estimate the tumor location may be a tumor centroid, landmark, feature and/or anchor, which may be a part of the tumor or anatomically correlated and/or co-localized with the tumor. One or more probability density functions that represent the likelihood of the defined tumor/target region landmark or tumor/target region anchor to be at different locations may be generated by simulating different shifts of the tumor/target region anchor to the different locations. Alternatively, or additionally, the one or more probability density functions may be generated from imaging data acquired during the treatment planning process. For example, treatment planning may comprise acquiring PET imaging data of the target region(s), which may be acquired on a diagnostic PET imaging system and/or on a radiotherapy system with an on-board PET imaging system. In some variations, PET imaging data of the target region(s) may be acquired on the radiotherapy system with an on-board PET imaging system, and may be acquired using similar acquisition settings as PET imaging data acquisition that occurs during a treatment session. For example, during a treatment session, there may be a plurality of limited or shortened acquisition time windows for detecting PET imaging data at each beam station, resulting in “limited time sampled” or LTS images acquired at a plurality of beam stations. These LTS images may be low-photon count images, possibly with low signal-to-noise ratios, low photon count, low contrast from background, blurry or poorly-defined edges, etc. In some variations, the PET LTS images may be reconstructed using time-of-flight filtered back projection (TOF-FBP), for example, single-slice rebinning and filtered back projection (SSRB-FBP). During treatment planning, the PET imaging data of the target region(s) may be acquired in a similar manner and used to generate probability density functions for plurality of target region shifts and locations. The probability density functions may be stored in a look up table that is in machine readable memories of the controller and/or processor.

The probability density functions may be in the image space or may be in the sinogram space. As used herein, “sinogram space” refers to a data domain in which tomographic acquisition measurements are parameterized by projection geometry rather than object spatial coordinates. In sinogram space, each datum represents the measured response along a defined ray path (e.g., a line of response, “LOR”) characterized by one or more projection parameters such as angle, radial offset, detector-pair index, projection index, etc. In time-of-flight (TOF) PET systems the ray path definition may also include a temporal offset. By contrast, “image space” denotes a domain indexed by spatial coordinates (e.g., pixel or voxel positions in x, y, z), where each value corresponds to an estimate of an object property (e.g., tracer activity concentration or attenuation) at a specific location. Sinogram space typically contains projection data organized by image acquisition geometry, whereas image space contains reconstructed object representations. A PDF in image space may indicate the probability or likelihood(s) of a target region shift or location in terms of spatial coordinates (e.g., coordinates in x, y, z), while a PDF in sinogram space indicates the probability or likelihood(s) of a target region shift or location in terms of detection or projection parameters (e.g., angle, offset).

FIG. 3B depicts a flowchart representation of one variation of a method for generating a plurality of probability density functions. Method 320 may comprise acquiring 322 limited time sampled (e.g., low-photon count) images of a target region over multiple patient platform beam stations, correcting, reducing, and/or removing 324 motion blur artifacts from the acquired images to obtain reduced-motion images, aligning 326 the reduced-motion images to a selected or defined target region location, deriving 328 a probability density function by combining the aligned reduced-motion images, and generating 330 a plurality of probability density functions by simulating a plurality of different target region shifts. The probability density functions may be stored in a look up table that is in machine readable memories of the controller and/or processor. The selected or defined target region location may be a portion of the tumor or target region defined by a clinician, and may be a target region centroid, landmark, feature and/or anchor, and/or part of the target region or anatomically correlated and/or co-localized with the target region. For example, the defined target region location may be the centroid of a planning target volume (PTV) or a target region anchor point where a PET signal has an elevated level of intensity (e.g., a standard uptake value or PET image pixel intensity above a predetermined threshold). The selected or defined target region location may be an initial location of the target region at the start of the image acquisition procedure, which may serve as a reference location from which target region shifts may be calculated. In some variations, aligning 326 the reduced-motion images may comprise aligning the images to a location selected in an image, e.g., a CT image, PET image, or any planning image.

Combining the reduced-motion images to derive a probability density function may comprise stitching the images together and/or overlaying the aligned images and summing them together. In some variations, the images may have different but adjacent, or overlapping, fields of view because they were acquired at different beam stations. These images may be stitched together to create a field of view broader than an individual image, e.g., the field of view may span across multiple beam stations. When combined, the pixel (or voxel) intensities of the aligned reduced-motion images sum together to represent the probability that a target region is in a particular location. For example, a PET imaging data of a target region that has taken up a PET tracer may have higher pixel or voxel intensities in the location of the target region, and lower pixel or voxel intensities elsewhere. Elevated PET imaging intensity values may be correlated with a higher probability that the target region is at a location while lowered PET imaging intensity values may be correlated with a lower probability that the target region is at a location. These PET images with different pixel or voxel intensities may be used to derive 328 a probability density function. Without wishing to be bound by theory, each probability density function may be considered a pre-defined, shifted template of what the target region looks like when it is in specific positions within the BTZ. Alternatively, or additionally, a probability density function may be derived 328 from a location uncertainty model (e.g., a location uncertainty cloud). The location uncertainty model may be calculated from measurements and/or simulations that characterize the performance (e.g., precision) of determining the target region location. In some variations, the peak or high values of the uncertainty model may be at the location with the highest likelihood of being the correct location of the target region. For example, the uncertainty model may be centered on the location of the target region. The uncertainty model may have a certain spread and average values for different potential locations of the target region, or may be consistent across all potential locations. The uncertainty model may be derived by applying the location estimator algorithms to a set of imaging data with known target region motion to calculate the estimated locations of the target region at different points in time/motion phases, and compare the estimated locations with the known target region locations. The difference between the estimated locations and the known “ground truth” locations may be used to derive the uncertainty model. Additional details of generating a location uncertainty model (e.g., location uncertainty cloud) are provided below.

A map of these probabilities over the pixels (or voxels) of the aligned reduced-motion images comprises a probability density function. In some variations, aligning 326 the reduced-motion images may comprise superimposing the reduced-motion images so that the target region in each of the images are centered or aligned with an initial image of the target region. Alternatively, or additionally, the method may comprise identifying the contour of the target region in each reduced-motion image, and then aligning and combining the images by superimposing the images such that the contours of the target region in each image are aligned with each other. One variation of the method 320 may comprise generating a stitched image of the acquired images of the target region (e.g., by aligning adjacent images and fusing the images at their boundaries) and reducing motion blur artifacts in the stitched image. The resultant stitched image may be used or converted into a probability density function. Generating 330 a plurality of probability density functions for a plurality of different shifts of the target region may comprise generating a first probability density function corresponding to a first shift by shifting the derived probability density function from step 328 by the first shift, and generating a second probability density function corresponding to a second shift by shifting the derived probability density function from step 328 by the second shift. This may be repeated for as many shifts as desired, for example, to as many shifts as needed to provide a desired coverage of a tumor's motion envelope or trajectory. The number of shifts and corresponding probability density functions may be determined at least in part on the desired precision level at which a target region's location is to be estimated. If a greater level of precision is desired (e.g., where the location estimation is precise up to a certain number of pixels or a certain resolution), then the method may comprise generating a larger number of probability density functions for a larger number of possible shifts. If a lower level of precision is desired, the method may comprise generating a smaller number of probability density functions for a smaller number of possible shifts. In some variations, the number of shifts and corresponding probability density functions may be determined by the size of the target region motion envelope or trajectory, and the distance interval between the shifts. Once the desired number of probability density functions have been generated for the desired number of target region shifts, they may be stored in a controller memory that may be accessed by the treatment planning system and/or treatment delivery system (i.e., radiotherapy system) during a radiation delivery session. During a radiation delivery session or radiotherapy treatment session, additional images of the target region(s) and associated location data of the target region(s) acquired during the session may be used to generate additional probability density functions for different target region shifts. For example, the set of probability density functions used in method 300 may be solely generated using imaging and location data acquired during a radiation delivery session or radiotherapy treatment session, or also may include probability density functions derived during treatment planning. Alternatively, the set of probability density functions used in method 300 may be solely generated during treatment planning.

In some variations, the generated plurality of probability density functions in step 330 are in the image space. Optionally, method 320 may comprise generating 332 sinograms for the plurality of probability density functions by forward-projecting the probability density functions. The sinograms of the plurality of probability density functions may also be referred to as probability density functions in sinogram space.

Reducing or removing 324 motion blur artifacts in the acquired images may use one or more of the deblurring methods described herein. For example, reducing the motion blur artifacts in the acquired images may use principal component analysis methods, as described further below.

In some variations, the acquired images may be high-quality images, such as PET imaging data with high signal-to-noise ratios, high photon count, high contrast from background, well-defined edges, etc. In some variations, the acquired images may include time-of-flight (TOF) data. Time-of-flight PET data may comprise listmode data of the detected positron annihilation photons, comprising time stamps indicating when photons (e.g., photons emitted from the positron annihilation event) were detected by the PET imaging system. Such timing data may be used to generate enhanced or clearer PET images, and/or may be used to calculate the location of a target region. In variations where the acquired images of the target region are of sufficient quality to identify the location and/or contours of a target region, it may not be necessary to remove any motion blur artifacts or apply additional image processing before aligning the images for deriving a probability density function.

Some variations of location estimation methods using statistical inference (e.g., MLE location estimation methods) may use backprojection PET images generated from TOF PET data. However, such statistical inference methods presume that the data points (e.g., voxels in the image) are statistically independent. FIG. 3D depicts an example of a TOF PET backprojection image of 3 LORs. A single LOR fills many pixels of a PET backprojection image, where each pixel of the LOR is correlated to the adjacent pixel of the LOR and may form a continuous line. The plurality of pixels of a single LOR may represent a single positron annihilation event, and not a plurality of uncorrelated positron annihilation events. Based on statistical inference methods, the correlation between pixels of an LOR may give the inaccurate impression that multiple positron annihilation events occurred (e.g., one event per pixel), even though it is just a single positron annihilation event. This may impact the precision of confidence metric values and/or location uncertainty estimates of this location estimation method.

Some variations of MLE methods for target location estimation may use an image of the origin point of a positron annihilation event calculated from TOF PET data, instead of a backprojection image. For example, the TOF PET data may be used to generate lines-of-response data (i.e. LOR) and the origin location point (e.g., a center point along the LOR, any point along the LOR) on the LOR where the positron annihilation event occurred may be calculated using the TOF time stamp data. In some variations, there may be some uncertainty about the exact location of the positron annihilation event, which may result in an uncertainty cloud of locations where the annihilation event may have occurred. In this example, the origin point may be selected as the point that is in the center of the uncertainty cloud of locations, or may be any point that is within the uncertainty cloud of locations. The origin points for a collection of LORs may form a cluster at a location where a target is likely to be located. FIG. 3E depicts an example of an image of LOR origin points (e.g., center or origin location points of the 3 LORs depicted in FIG. 3D). Some variations of a location estimation method that uses statistical inference (e.g., MLE) may comprise calculating the likelihoods (e.g., probabilities) that the cluster of LOR centers in the image arises from a target region located at set of potential locations, and selecting the target location from the set of potential locations that has the greatest likelihood (e.g., highest probability). Without wishing to be bound by theory, a MLE location estimation method finds the solution to the question “What is the probability that a PET-avid tumor (or target region) at a particular location would result in the pattern of positron annihilation location points in the image?” for a plurality of possible tumor/target locations. Alternatively, or additionally, the MLE location estimation method may comprise calculating the probability that the pattern of LOR origin points in the image would occur for a number of potential tumor/target region locations. In some variations, TOF PET data acquired in limited or shortened acquisition time windows (e.g., at each beam station) may be used to generate an image of LOR origin points (or centers) instead of generating an LTS image. The image(s) of LOR origin points may be used to calculate a radiation fluence for delivery instead of using LTS image(s). While variations may not use LTS images to calculate the radiation fluence for delivery, they may still generate LTS images as may be desirable (e.g., for generating a full image periodically during radiation delivery, and/or at the end of a radiation delivery session).

FIG. 3C is a flowchart representation of one variation of a statistical inference method (e.g., maximum likelihood estimation) that may be used to calculate a location of a target region from TOF PET imaging data. In some variations, the imaging data used in the MLE described herein may comprise PET imaging data comprising time-of-flight (TOF) data from which the origin location of the positron annihilation photons (i.e., the location where the positron annihilation event occurred) may be calculated. Method 340 may comprise acquiring 342 TOF PET data of a target region, calculating 344 lines-of-response (LORs) from the acquired TOF PET data, determining 346 the origin locations of the calculated LORs to form an image of LOR origin points, computing 348 the likelihood of the image of LOR origin points for different target region shifts, identifying 350 the target region shift with the greatest likelihood value and/or exceeds a threshold value, and generating 352 an estimated location of the target region by applying the identified target region shift to the initial/previous location of the target region. In some variations, computing 348 the likelihood of the image of origin points may comprise using probability density functions for each target region shift, such as the probability density functions generated using method 320 and described in FIG. 3B. The probability density functions may be in image space or sinogram space. Computing the likelihood of the image of origin points may comprise applying a probability density function corresponding to a plurality of target region shifts or locations to the acquired images. In some variations, this may comprise cross-correlating the plurality of probability density functions with the acquired images or imaging data.

While some variations of a statistical inference method (e.g., maximum likelihood estimation) that may be used to calculate a location of a target region may be implemented in image space, other variations may be implemented in sinogram space. As described above, an LOR represented in image space may fill many pixels in the image, in which adjacent pixels are not uncorrelated because they form a continuous line. However, in sinogram space, a LOR may fill a single pixel or possibly 2 pixels (i.e., one pixel for each detected coincident photon of a photon pair that may define an LOR). Such pixels may be considered statistically independent, which may improve the accuracy of an MLE method for estimating the location of a target region. However, because there may be positron annihilation events that arise from non-target region tissue, e.g., background PET activity, the location estimation methods may comprise a filter or mask that eliminates PET data and/or LORs that do not intersect the target region and/or a motion envelope of the target region. For example, a biology targeting zone (BTZ) may be a region around the target region that acts as a filter or mask such that PET data that does not originate from an annihilation event within the BTZ is excluded from the location estimation calculation. The BTZ may be defined in image space and/or may be defined in sinogram space. A BTZ in sinogram space may be used with a statistical inference method (e.g., MLE) for computing the estimated target location in sinogram space. Without wishing to be bound by theory, computations in sinogram space may be desirable because such computations may be less impacted by artifacts from backprojection image reconstructions methods, and/or the points in a sinogram plot have more statistical independence from each other as compared to pixels in an image, which may improve the accuracy of a statistical inference method such as MLE. Computing the target region location using statistical inference methods in sinogram space may preserve the statistical independence of the points in the sinogram plot without requiring TOF PET data. For example, such methods may be used with PET LOR data that does not contain TOF PET data.

FIG. 3F is a flowchart representation of one variation of a statistical inference method (e.g., maximum likelihood estimation) that may be used to calculate a location of a target region from PET imaging data. In this variation, the calculations are performed in sinogram space and the method may comprise filtering or masking out PET imaging data originating from positron annihilation events that are outside a defined region, for example, outside a biology tracking zone (BTZ). The PET imaging data in this method may comprise TOF PET data or may not comprise TOF PET data. Method 360 may comprise acquiring 362 PET imaging data of a target region, calculating 364 lines-of-response (LORs) from the acquired PET imaging data, extracting 366 LORs that intersect with the biology tracking zone (BTZ), generating 368 an image of the extracted LORs in sinogram space, computing 370 the likelihood of the sinogram image of the LORs for different target region shifts, identifying 372 the target region shift with the greatest likelihood value and/or exceeds a threshold value, and generating 374 an estimated location of the target region by applying the identified target region shift to the initial/previous location of the target region. The acquired PET imaging data may comprise TOF PET data or may not comprise TOF PET data. Extracting 366 LORs that intersect with the BTZ may comprise excluding LORs that do not intersect with the BTZ. Computing 370 the likelihood of the sinogram image of the LORs for different target region shifts may use probability density functions in sinogram space for each shift. For example, computing the likelihood of the sinogram image of the LORs may comprise applying a probability density function corresponding to a plurality of target region shifts or locations to the sinogram image. In some variations, this may comprise cross-correlating the plurality of probability density functions with the sinogram image. The probability density functions for a plurality of different shifts of the target region may be generated using any method, such as method 320 depicted in FIG. 3B.

MLE with Nearest Neighbor Weighted Averaging

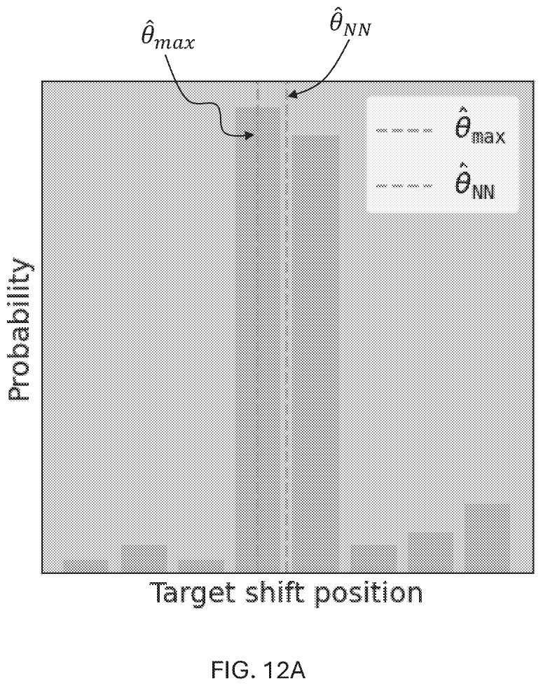

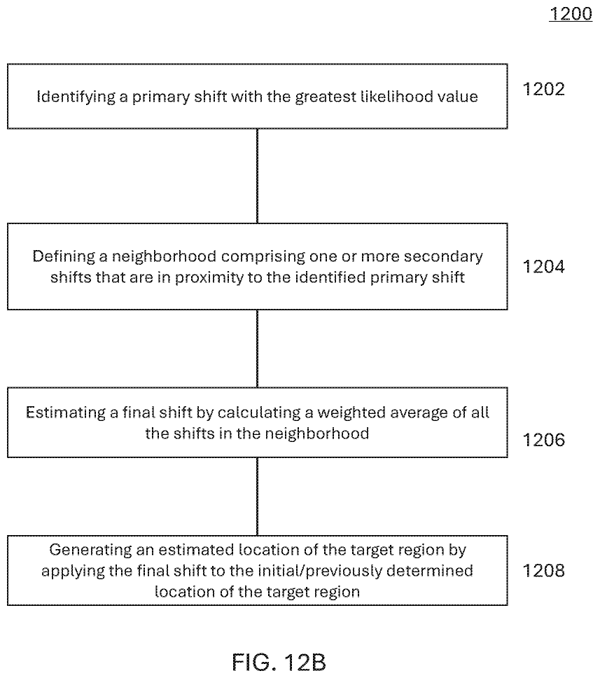

The methods described in FIGS. 3A, 3C, and 3F generate an estimate of the target region location by selecting a shift (or location) that has the maximum likelihood of resulting in the acquired PET imaging data, in either image space or sinogram space, and applying that shift to a previously-calculated location estimate of the target region. This is performed using a set of probability density functions (PDFs) that correspond to a set of potential target region shifts. The set of potential target region shifts may be selected or pre-determined by the user or controller based on the computational bandwidth or complexity of the controller(s) and/or processor(s); that is, the larger the set of potential target region shifts, the greater the number of PDFs that need to be applied to the PET imaging data, and the greater amount of time or computational bandwidth needed. There may be a tradeoff between the granularity/resolution of the shift (or location) and the amount of time and/or processor bandwidth that may be allocated to calculating a set of likelihood values and to identify one or more likely target region shifts. In other words, the set of potential target region shifts (or locations) may be a finite, discrete number; the target region shift values may be discretized by the selected set of potential target region shifts (or locations). However, it is possible that in some situations, the target region may have shifted by a value that is in between the discrete shift values of the set, and it may be desirable to be able to identify that in between shift value in order to generate a more accurate estimate of the target region location. One variation of an MLE target location estimation method may comprise calculating a weighted average of the target region shifts in a neighborhood of the shift with the maximum likelihood value to attain a shift value that is between the shift values in the set of potential, discrete shift values. FIG. 12A is a conceptual depiction of a location estimation method using MLE with nearest neighbor weighted averaging. For example, a MLE location estimation method with nearest neighbor weighted averaging may comprise identifying a primary shift (\hat{θ}ₘₐₓ) in a set of discrete shifts that has that maximum (e.g., largest, best, threshold-exceeding) likelihood value, selecting one or more secondary (e.g., “neighbor”) discrete shifts in the set of discrete shifts that is near the primary shift, and calculating an average of the primary and secondary shifts by weighting the shifts according to their respective likelihood values to attain a final shift (\hat{θ}_{NN}) that is between the discrete shifts in the set. Alternatively, or additionally, selecting the one or more secondary discrete shifts may comprise selecting the shifts that have likelihood values that are near the likelihood value of the primary shift.

This method may allow for the calculation of a target region shift value that is not limited to the discrete, pre-selected shifts in the set of potential target region shifts, and may facilitate a more precise and/or accurate target region location estimate. This method may address discretization issues where adjacent regions or shifts may have similar probabilities of the tumor being located there where simply selecting the largest correlation value may be less precise because the location could fall between two discrete regions (as conceptually depicted in FIG. 12A); this method may help calculate the true position instead of being forced to select a single shift. In some situations, there may be many neighboring shift positions which are equally likely to be tumor shift (or location). For example, there may be multiple shifts in a set of potential target region shifts that have high probability or likelihood values. In such a scenario, the target may be most likely be in the middle of these shifts (or locations). MLE with nearest neighbor weighted averaging may be able to arrive that that “in-between” shift value. Using nearest neighbor weighted averaging may generate a more precise target shift or location estimate while using less computational resources than having a larger set of potential target region shifts, which may increase the number of likelihood values that need to be calculated and stored in controller and/or processor memory.

FIG. 12B depicts one variation of an MLE target location estimation method that uses nearest neighbor weighted averaging. Method 1200 may comprise identifying 1202 a primary shift (i.e., of the target region) with the greatest likelihood value, defining 1204 a neighborhood of one or more secondary shifts that are in proximity to the identified primary shift, estimating 1206 a final shift by calculating a weighted average of all the shifts in the neighborhood, and generating 1208 an estimated location of the target region by applying the final shift to the initial (or previously determined) location of the target region. Identifying 1202 the primary shift may use any of the methods described previously, e.g., as described and depicted in FIGS. 3A, 3C, 3F. In some variations, the primary shift may be the target region shift with a likelihood value that exceeds a predetermined threshold and/or one or more other criteria. Defining 1204 a neighborhood may comprise selecting one or more shifts in the set of potential target region shifts that are closest in value to the primary shift. For example, the neighborhood may comprise the two shifts in the set that are adjacent to the primary shift, e.g., one shift value greater than the primary shift value and one shift value less than the primary shift value. The neighborhood may comprise three or more shifts, e.g., two shifts greater than the primary shift value, and one shift less than the primary shift, or vice versa, and so on, with 4 or more shifts, 9 or more shifts, 12 or more shifts, etc. The number of potential target region shifts in a neighborhood may be determined by a user and/or by a clinic's desired setting or protocol. Alternatively, or additionally, the neighborhood may comprise one or more shifts that have likelihood values that are in proximity to the likelihood value of the primary shift. Estimating 1206 the final shift may comprise deriving the weight for each shift from its corresponding likelihood value, and using the derived weight to compute the final (e.g., average) shift. In some variations, the weight for each shift may be the likelihood value for that shift. In other variations, the weight for each shift may be the likelihood scaled by a factor or the likelihood normalized against the likelihood value of the primary shift (e.g., normalized against the maximum likelihood value), and/or the weights may be the likelihood values normalized to 1 (e.g., such that the weights sum to 1). One variation of calculating the weighted average may comprise multiplying each shift value in the neighborhood by its weight to obtain a shift product, summing the shift products across the neighborhood, and dividing the summed shift products by the number of shifts in the neighborhood. Generating 1208 the estimated location of the target region may be performed using any of the methods described herein, for example, as described above in FIGS. 3A, 3C, and 3F.

### Time-of-Flight (TOF) PET Location Estimation Methods

Another algorithm or method for estimating the location of a target region for calculating a radiation fluence may comprise the use of time-of-flight (TOF) PET imaging data. Time-of-flight PET data may include sufficient time difference information to calculate the location of the positron annihilation event along a detected line-of-response (LOR). In some variations, time-of-flight PET data for a plurality of lines-of-response defined by a plurality of coincident photon pairs may comprise timing data of when each photon in a pair of coincident photon pairs was detected by the PET detectors. A PET tracer injected into a patient and taken up by a tumor will emit positrons which will annihilate with a nearby electron. The positron annihilation event will generate two 511 keV photons that move in opposite directions, forming a line-of-response. The two photons (which may be referred to as coincident photons or a photon pair) may be detected by PET detectors located opposite to each other. The PET detectors configured for time-of-flight PET may have sufficient sensitivity and/or precision to detect the exact time in which each of the photons strikes the detector within the coincidence window (a window of time within which detected photons are considered coincident). The time difference in which coincident photons are detected may be used to calculate an estimate of the location of the positron annihilation event along the line-of-response. In some variations, the time-of-flight PET detectors may have some degree of timing imprecision, which corresponds to a similar degree of location imprecision. This location imprecision may be used to expand one or more target region contours (e.g., a biology tracking zone in biology-guided radiotherapy) to ensure that PET signal from the tumor is not disregarded. The location imprecision may be represented as a probability cloud centered on the estimated location of the positron annihilation emission event, where the size of the probability cloud is correlated with the timing imprecision with which the PET detectors detect photons. PET detectors may acquire time-of-flight PET data for a plurality of lines-of-response. The system controller may calculate the estimated location of the positron annihilation event for each of the lines-of-response using the time-of-flight PET data. The plurality of locations of the positron annihilation events may comprise a distribution or pattern of positron annihilation event locations. In some variations, this distribution or pattern of positron annihilation events may include the probability clouds for the location of each positron annihilation event. Regions of the PET image where there is a higher density of positron annihilation event locations and/or greater overlap of probability clouds may have an increased likelihood of being the tumor or target region. The distribution or pattern of positron annihilation events may be considered a probability map representing the likelihood that the target region is in a particular location. For example, the distribution or pattern of positron annihilation events may be considered as a probability density function. In some variations, the estimated location of the target region may be a centroid of the distribution or pattern of positron annihilation events.

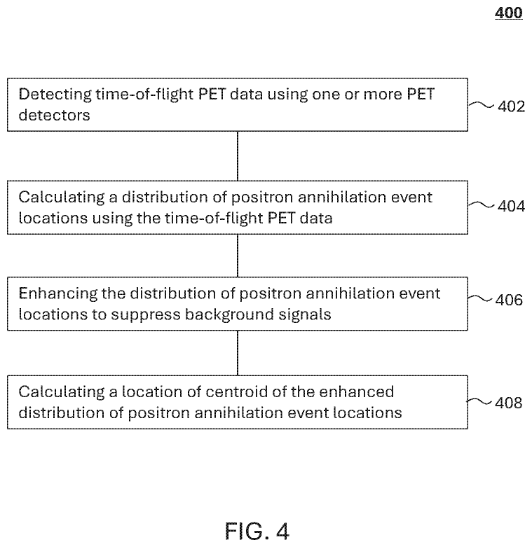

FIG. 4 is a flowchart depiction of one variation of a method that may be used to calculate a location of a target region using time-of-flight PET data. Method 400 may comprise detecting 402 time-of-flight PET data using two or more PET detectors, calculating 404 a distribution (or pattern) of positron annihilation event locations using the time-of-flight PET data, enhancing 406 the distribution of positron annihilation event locations to suppress background signals and/or enhance signals that are likely to originate from the target region(s), and calculating 408 a location of centroid of the enhanced distribution of positron annihilation event locations. Calculating 404 a distribution of positron annihilation event locations may comprise calculating, for each detected line-of-response, the location along the line-of-response where the positron annihilation event occurred (optionally, with a probability cloud representing a range of locations based on the uncertainty of PET detector time-of-flight data). For a plurality of lines-of-response, there may be a plurality of positron annihilation event locations, which may comprise a distribution or pattern of positron annihilation event locations. These may be collated together onto a plot that may resemble an image or scatterplot. Enhancing 406 the distribution or image of the positron annihilation event locations may comprise one or more of applying a difference of Gaussians filter to the distribution or image to sharpen edges, enhance the signal from the target region, and/or suppress or remove noise (e.g., background noise). Alternatively, or additionally, enhancing 406 the distribution or image of positron annihilation event locations may comprise applying an uncertainty kernel (e.g., a function that represents the uncertainty of the time-of-flight PET data) to the distribution or image to expand the possible positron annihilation event locations, and in turn, expand or include the possible location(s) of the target region(s). Optionally, the contours of the target region(s) and/or motion envelopes (e.g., biology tracking zone) may be adjusted according to the enhanced distribution or image of the positron annihilation event locations. In some variations, the enhanced image of the positron annihilation event locations and/or the location of the centroid may be backprojected to the firing positions of the therapeutic radiation source. The location of the centroid may be the estimated location of a target region. The estimated location of the target region as calculated 408 may be used in any of the methods herein (e.g., in the methods of FIGS. 2A-2C) to determine a final estimated location of the target region. The final estimated location of the target region may be used to calculate a radiation fluence for delivery to the target region, as described above.

In some variations, the method 400 may be used to process time-of-flight PET data acquired during treatment planning, where no therapeutic radiation is delivered to the target region(s). During a treatment planning imaging session, time-of-flight PET data is acquired as a target region moves, and method 400 may be used to calculate centroid locations during the target region motion. The time-of-flight PET data in conjunction with the centroid locations may be used to generate motion models for the target region, and/or to test any of the location estimation methods or algorithms described herein. For example, the distributions or images of positron annihilation event locations may be used to simulate PET imaging data that may be acquired during a treatment session. The calculated location of the centroid may be considered as “ground truth” for the target region location. Any of the target region location estimation methods described herein may be applied to these distributions or images to generate an estimated location. The estimated locations may be compared with the “ground truth” locations (e.g., the calculated centroid locations), and the comparison may be used to determine a confidence metric and/or uncertainty model that may reflect the accuracy and/or precision of the location estimation method.

Since time-of-flight PET imaging data uses precise detection data of photons and timing resolution provided by the PET detectors, the error of the estimated location of the target region may depend greatly on the function (e.g., sensitivity) of the PET detectors. PET detectors that are included onboard with a radiotherapy system may be susceptible to afterglow effects arising from the high-energy photons emitted by the therapeutic radiation source (e.g., linac). These afterglow effects may compromise the accuracy of the time-of-flight timing data and timing discrimination between the reception/detection time of coincident photons. As such, some variations of radiotherapy systems having on-board PET imaging systems may also comprise components that measure indicia of PET detector afterglow effects. For example, the bias current levels of PET detectors, temperature, dark count rates, ambient radiation levels and/or energies, and/or radiation output (e.g., pulses) of the therapeutic radiation source, etc. may be indicative of the amount of afterglow artifact impacting the PET detectors. If high levels of afterglow are detected by the radiotherapy system, in some variations, the controller may not use the time-of-flight method to estimate the location of the target region, and/or may use other location estimation methods or algorithms to confirm or check the time-of-flight location estimation method. For example, one method may comprise using time-of-flight PET data at the beginning of a radiation delivery session when PET detector afterglow effects are less likely to impact the precision, sensitivity, and/or accuracy of the PET detectors, e.g., when the time-of-flight PET data has the highest likelihood of being accurate and precise. If and when afterglow effects on the PET detectors exceed a predetermined threshold and/or are outside an acceptable operating range, the method may alternatively or additionally use location estimation methods or algorithms that do not rely on time-of-flight PET data. In one variation, time-of-flight PET data may be used to estimate the location of a target region at the beginning of a treatment session, and when the afterglow impact on PET detectors exceeds a threshold, the system may use PET LOR data without time-of-flight data with MLE methods and/or correlation methods, alone or in combination with the method of FIG. 4, to estimate the location of the target region. Adjusting the location estimation methods or algorithms as desired within a treatment session or radiation delivery session, in response to the quality of acquired imaging data, imaging (e.g., PET) detector function, and/or changing operating conditions may help facilitate more accurate estimations of the target region(s), which may in turn promote the conformal delivery of radiation to the target region(s).

### Adaptive Correlation Filter Location Estimation Methods

Another algorithm or method for estimating the location of a target region may comprise applying a correlation filter to imaging data of the target region. The estimated location may then be used to calculate a radiation fluence for delivering a conformal radiation dose to the target region, where the radiation dose may be less susceptible to noise from the imaging data. In one variation, a method may include acquiring imaging data of a target region, generating a filtered image by applying a tracking filter (such as an adaptive correlation filter) to the imaging data, determining a target region location from the filtered image, and calculating a radiation fluence to be delivered to the target region using the target region location. In some variations, the location of the target region may be the location of a target anchor. A target anchor may be the centroid of the target region or any selected or user-defined landmark and/or feature and/or structure that is associated with the target region, and whose location is used to represent the location of the target region. A tracking filter (which may also be referred to as a tracking template), such as an adaptive correlation filter, may be generated during a treatment planning session using one or more planning images, a target region contour defined on the planning images, and selected target anchor location. The tracking filter may be applied to imaging data acquired during a radiation delivery session (e.g., radiotherapy session) to calculate an estimate of the real-time location of the target region(s). The estimated location of the target anchor location may be used to update the radiation fluence for delivery, which may help ensure that the prescribed radiation is delivered to the actual location of the target region, while limiting the impact of imaging data artifacts on the dose delivered to the target region or patient. In some variations, an adaptive correlation filter may optionally be updated during the treatment session (e.g., at the start or multiple times throughout the treatment session), which may help further refine its estimation of the target anchor location (e.g., improve the accuracy and/or confidence of identifying the target anchor location). Additional details on variations of a correlation filter and adaptive tracking filters may be found in PCT Application No. PCT/US2023/079652 filed Nov. 14, 2023, which is hereby incorporated by reference in its entirety.

In some variations, applying the tracking filter to imaging data may comprise convolving the tracking filter with imaging data. The adaptive correlation filter may be an imaging template of a target region that may be applied to imaging data to identify the target region within that image. Identifying the target region using an adaptive correlation filter may include determining the location of the target region within an image that has been filtered by the adaptive correlation filter. For example, in some variations, using an adaptive correlation filter to process imaging data may generate an estimated location of a target region anchor, e.g., tumor centroid or any user or controller/processor selected location. In other words, filtering or processing the imaging data using an adaptive correlation filter may generate a filtered image that has a high-intensity region at the location of the target anchor location. Conceptually, applying an adaptive correlation filter template to imaging data may be similar to pattern matching; the template may be shifted across the imaging data to find the region of the image that correlates most strongly with the “template” pattern (e.g., highly correlated with an image of the tumor). Once the region of the image that correlates strongly with the planning image of the tumor, that region is “highlighted” while regions with lower correlation are suppressed or ignored. In other words, the noise and/or artifacts in the original imaging data may be eliminated or suppressed in the filtered image of the target anchor location. The anchor of a target region is a location that is defined relative to a contour of the target region during treatment planning. During treatment planning, firing filters may be generated based on CT imaging data, PET imaging data, and/or one or more images of the target region anchor (e.g., a Gaussian or delta function or any similar or desired distribution, centered over the defined location of the target region anchor). An adaptive correlation filter may also be generated during treatment planning. During radiation delivery, the firing filters may be applied to the estimated location of the target anchor to generate a radiation fluence for delivery. In some variations, the firing filters may be applied across multiple firing positions of the therapeutic radiation source to projections of the images of the target anchor location to that firing position. The estimated location of the target anchor may be determined from imaging data that has been filtered (and/or cross-correlated) with the adaptive correlation filter, and an image of the target anchor location may comprise a Gaussian or delta function centered over the estimated location of the target anchor. Because the firing filters are applied to an image of the target anchor location (e.g., applied to a projection of the image of the target anchor location on the respective firing position), and not to the imaging data directly, the resultant radiation fluence may be less susceptible to the effects of imaging noise and artifacts. In some variations, the image of the target anchor location may be a de-noised version of the filtered image, where the region of the image that corresponds with the location of the anchor is sharpened or intensified, and the image noise is suppressed. The image of the target anchor location may be a combination of empirically acquired imaging data and simulated imaging data, for example, a Gaussian function (simulated data) centered over the location of the anchor and suppressed imaging noise from the acquired imaging data. Alternatively, or additionally, the planned radiation fluence may be shifted according to the estimated location of the target region anchor. Moreover, in some variations, the adaptive correlation filter may be trained with additional imaging data (i.e., further refining the imaging template) so that it provides a better estimate of the target anchor location. The additional imaging data may be imaging data acquired during the beginning of a treatment session, e.g., PET prescan imaging data, or earlier on the same day as the treatment session.

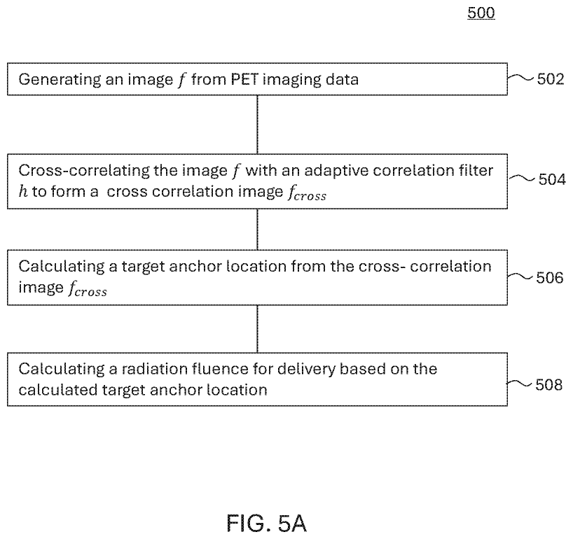

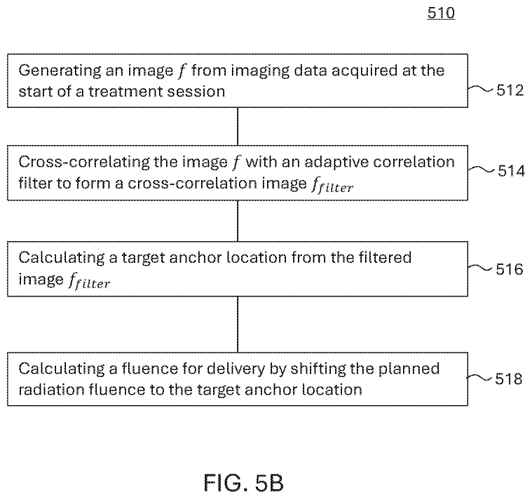

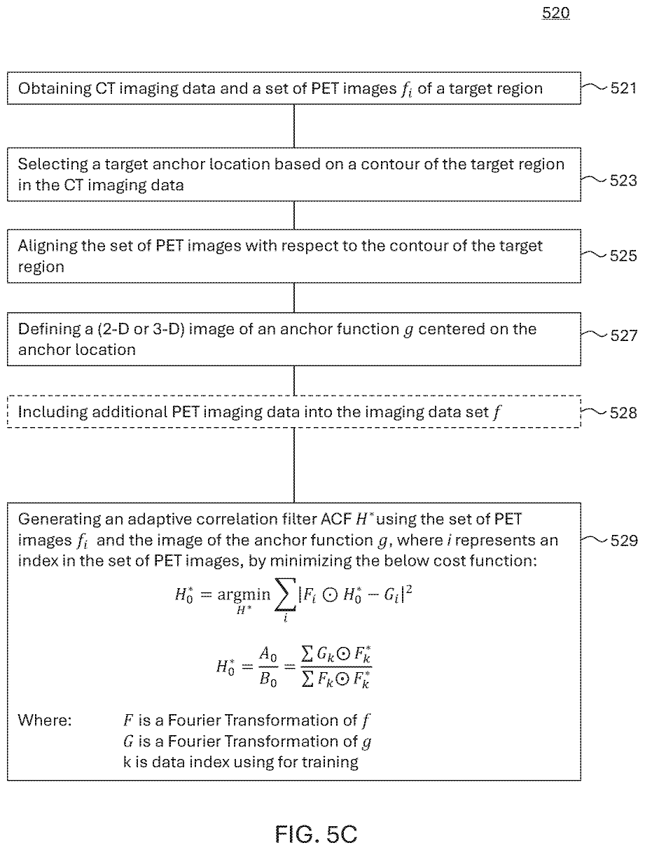

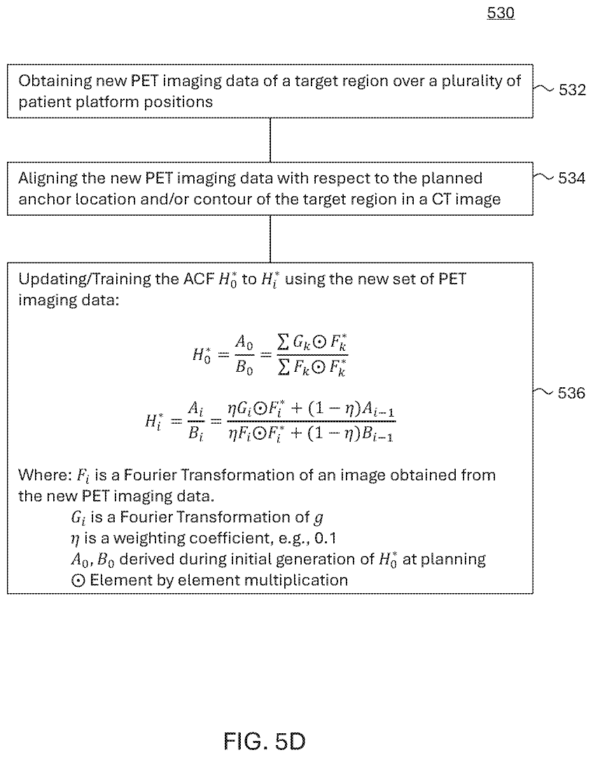

FIG. 5A depicts a flowchart representation of one variation of a method for calculating a target anchor location and radiation fluence for delivery using an adaptive correlation filter using PET imaging data acquired during a treatment session. The method 500 includes generating 502 an image f from acquired PET imaging data, cross-correlating 504 the image f with an adaptive correlation filter to form a cross correlation-image, calculating 506 a target anchor location from the cross-correlation image, and calculating 508 a radiation fluence for delivery based on the calculated target anchor location. The PET imaging data may be acquired at a particular beam station or control point, e.g., patient platform position. The image f may be a two-dimensional matrix as described above. Optionally, method 500 may comprise removing noise from the image f, for example, using a generative adversarial network (GAN) and/or one or more filters such as low-pass filters. In some variations, cross-correlating 504 the image f may comprise cross-correlating the image matrix f with the ACF h. The cross-correlation image may also be referred to as a filtered image. In one variation, the target anchor location may correspond to indices i, j of f_{filter}(i, j) (herein, such indices are also referred to as coordinates) at which the filtered image has a maximum value (e.g., {i, j}→max (f_{filter}(i, j)). Calculating or determining 506 the target anchor location from the cross-correlation image may comprise by calculating a centroid of the filtered (i.e., cross-correlation) image and/or a centroid of a region of pixels or voxels that have an intensity value above a specified threshold. Alternatively, or additionally, calculating the target anchor location may comprise averaging the location of pixels that have an intensity value above a specified threshold. For example, calculating 506 the target anchor location may include identifying the high-intensity pixel(s) or voxel(s) in the cross-correlation image f_{filter}. The location of the target anchor may be the location of the highest-intensity pixel or voxel, and/or may be the location of the centroid of a cluster of high-intensity pixels or voxels. Method 500 may optionally comprise generating an image of the target anchor location, and using the image of the target anchor location to calculate 508 the radiation fluence for delivery. An image of a target anchor location may be a Gaussian function g (or a function similar to a Gaussian function, or a delta function) which may have a maximum value at (or in close proximity of) the target anchor location. In some variations, the image of a target anchor location may be an image of a delta function where the peak or maximum value is centered on the target anchor location determined from the cross-correlation image. Alternatively, or additionally, the image of a target anchor location may be an image having a plurality of delta functions where the peaks are in proximity to the target anchor location. The characteristics of the Gaussian function and/or delta function used to generate the image of the target anchor location may be the same as the Gaussian function and/or delta function used during treatment planning. For example, if treatment planning was based on an image of the target anchor that comprises a Gaussian function of a particular RMS width (or spread) and peak located over the location of the target anchor, generating an image of the target anchor location during radiation delivery may use the same Gaussian function with the same width and peak. In some variations, the image of the target anchor location may be a de-noised version of the filtered image, where the region of the image that corresponds with the location of the anchor is sharpened or intensified, and the image noise is suppressed. The image of the target anchor location may be a combination of empirically acquired imaging data and simulated imaging data, for example, a Gaussian function (simulated data) centered over the location of the anchor and suppressed imaging noise from the acquired imaging data.

For example, one variation of the method 500 may comprise acquiring imaging data (e.g. PET imaging data), generating a filtered image by applying (or cross-correlating) an adaptive correlation filter with the acquired imaging data, calculating the target anchor location by identifying the location(s) of the high-intensity pixels or voxels in the filtered image, generating an image of the target anchor location by using a Gaussian or delta function centered over the calculated target anchor location, and calculating the radiation fluence for delivery using the image of the target anchor location (e.g., by convolving the image of the target anchor location with firing filters, by convolving the projection of image of the target anchor location on the firing position with the corresponding firing filter for that firing position, shifting the planned radiation fluence to the target anchor location). Optionally, before applying the adaptive correlation filter to the acquired imaging data, the noise in the imaging data may be removed or reduced using, for example, a denoising GAN model (e.g., a GAN that has been trained on multiple prior images of the patient and target region(s)) and/or one or more noise-reduction filters.

In one variation, calculating 508 the radiation fluence for delivery may include applying a firing filter to the target anchor location and/or the image of the target anchor location. A firing filter may be a mapping function that converts imaging data (e.g., an image of the target anchor location) into a corresponding radiation fluence. The radiation fluence for delivery from a particular firing position may be calculated by applying the firing filter for that firing position to the projection of the image of the target anchor location onto that firing position. The firing filter may be calculated based on previously-acquired imaging data of the target region (e.g., CT imaging data, PET imaging data, and/or an image of the target anchor) during treatment planning, as described herein. In some variations, applying the firing filter to the target anchor location comprises convolving an image of the target anchor location (e.g., a Gaussian function and/or a delta function centered over the location of the target anchor) with the firing filters. Alternatively, or additionally, the firing filter may be applied by convolving the filtered image f_{filter} with the firing filter. An example description of calculating firing filters during treatment planning and applying the calculated firing filters to a suitable image (e.g., the filtered image f_{filter} or a PET image of the target region or an image of the target anchor location) is provided in U.S. Pat. No. 10,688,320 filed May 30, 2018, and International Patent Application No. PCT/US2021/044405, filed Aug. 3, 2021, which are hereby incorporated by reference in their entirety. In some variations, calculating the radiation fluence includes shifting a planned radiation fluence according to the target anchor location. For example, the planned radiation fluence may be defined relative to the target anchor location based on planning images during treatment planning. Prior to radiation delivery, the location of the target region and the target anchor location may have changed. The updated location of the target anchor may be calculated by applying the ACF to the imaging data, as described herein. Then, for the radiation delivery, the planned radiation fluence may be shifted to the calculated target anchor location. That is, the changes and shifts of the target anchor location from the planning phase to the treatment session may be applied to the planned radiation fluence so that the radiation is delivered to the updated location of the target. For example, if the target anchor location shifted by 1 cm, the radiation fluence for delivery may be calculated by shifting the planned radiation fluence by 1 cm. In some cases, the planned radiation fluence comprises a segmented multi-leaf collimator (MLC) leaf pattern and calculating the delivery radiation fluence comprises shifting the segmented MLC leaf pattern according to the calculated target anchor position. Alternatively, or additionally, calculating the radiation fluence for delivery may comprise using a lookup table that maps anchor target locations to corresponding MLC leaf patterns, and updating the MLC leaf pattern based on the lookup table data. In some variations, the methods described herein may be used to track changes in the target anchor location before and/or during a treatment session, and then apply those changes (e.g., positional shifts) to the radiation fluence for delivery and/or machine instructions. Since the relative location between the target anchor and the target region contour may be defined during treatment planning, changes in the target anchor location may be used to estimate changes in the position of the target region. Adjusting the radiation fluence for delivery according to changes in the target anchor location may facilitate the delivery of the prescribed dose of radiation to the actual location of target region.

Optionally, the method 500 may further include delivering the calculated radiation fluence to the target region with a therapeutic radiation source.

In some variations, an adaptive correlation filter may be trained on imaging data acquired moments just before radiation delivery, and/or during an initial portion of the treatment session where imaging data is acquired to confirm the safety of proceeding with radiation delivery. The location of the target region (e.g., target anchor) may be used as a landmark to which the planned fluence map may be shifted. FIG. 5B is a flowchart depiction of one variation of a method for calculating a fluence for delivery using the estimated location of a target region. This method may be used during an intensity modulated radiation therapy (IMRT) and/or stereotactic body radiation therapy (SBRT) and/or biology-guided radiation therapy treatment session. In some variations of IMRT or SBRT treatment sessions, no PET imaging data is acquired and/or no biologically-related imaging data is acquired. Method 510 may include generating 512 an image f from imaging data (e.g., CT images) acquired during the SBRT/IMRT procedure, cross-correlating 514 the image f with an adaptive correlation filter to form a cross correlation image, calculating 516 a target anchor location from the cross-correlation image, and calculating 518 a radiation fluence for delivery based on the calculated target anchor location. Optionally, method 510 may comprise removing noise from the image f, for example, using a generative adversarial network (GAN) and/or one or more filters such as low-pass filters. The imaging data may be acquired at the start of the treatment session and may comprise, for example, CT imaging data acquired to localize the patient on the patient platform (i.e., register the location of the patient relative to the coordinate system of the radiotherapy system) and/or PET imaging data acquired as part of a prescan before treatment. The image f is a two-dimensional matrix as described above. In some variations, cross-correlating 514 the image f may comprise crosscorrelating—the image f with the ACF h. The cross-correlation image may also be referred to as a filtered image (and an image filtered by an adaptive correlation filter may be referred to as a cross-correlation image). i, j of fritter (i, j) (herein, such indices are also referred to as coordinates) at which the filtered image has a maximum value (e.g., {i, j}→max (f_{filter}(i, j)). Alternatively, any other suitable approaches may be used for determining the target anchor location based on the filtered image f_{filter}. For example, the target anchor location may be a centroid of the filtered image and/or a region of image pixels that have an intensity value above a specified threshold. In some variations, an image of the target anchor location may be generated using the cross-correlation image f_{filter}.

The method 510 further includes calculating 518 a radiation fluence to be delivered to the target region based on the calculated target anchor location. In one implementation, the radiation fluence may be calculated by applying a firing filter to an image of the target anchor location, as described above. Alternatively, or additionally, the radiation fluence may be calculated by shifting the planned radiation fluence to the target anchor location.

Optionally, the method 510 further includes delivering the calculated radiation fluence to the target region with a therapeutic radiation source.

During an IMRT or SBRT or biology-guided radiotherapy treatment session, imaging data may be acquired not just at the beginning of the treatment session, but optionally at one or more time points after radiation delivery has begun. In some variations, methods 500, 510 may be repeated multiple times during the treatment session, to multiple collections or boluses of imaging data. This may help to ensure that the target anchor location is routinely updated throughout the treatment session so that the delivered radiation tracks the actual location of the target region and artifacts and/or noise in the acquired imaging data have little or no impact on delivered dose conformality. For example, methods 500, 510 may be repeated after each patient platform shuttle pass, where imaging data acquired during the shuttle pass and/or at the end of the shuttle pass is used to update the location of the target anchor. The updated location of the target anchor may then be used to calculate the radiation fluence for delivery in the next shuttle pass. Optionally, some variations may comprise depicting a PET image at the end of a shuttle pass that generated from PET imaging data acquired during the shuttle pass. For example, at the start of the first shuttle pass, the controller may output the PET prescan image to a display device, at the end of the first shuttle pass and/or the start of the second shuttle pass, the controller may output a PET image generated from the PET imaging data acquired during the first shuttle pass, at the end of the second shuttle pass and/or the start of the third shuttle pass, the controller may output a PET image generated from the PET imaging data acquired during the first second pass, and so on.

A correlation filter or tracking filter may be calculated before the radiation delivery or radiotherapy treatment session, for example, during a treatment planning procedure. For example, an adaptive correlation filter may be generated using an initial set of imaging data and then further updated (i.e., adapted, trained) based on additional imaging data. For radiotherapy methods, the adaptive correlation filter may be generated based on target region contours that are defined using planning images (e.g., CT, MR, and/or PET images) and a target anchor (e.g., tumor centroid) for each target region, both defined at least in part by a clinician (with or without the aid of auto-contouring tools). For example, the target region contour and target anchor (including its location) may be defined on planning CT images. The relative position between the target anchor and the target region contour may be fixed during treatment planning. The treatment planning system may then calculate a planned fluence that is associated with the target region contour and is referenced to the target anchor. That is, the target anchor may be a feature that functions as a reference point or landmark, and the planned radiation fluence may be associated or “locked onto” the target anchor. During treatment delivery, the location of the target anchor may be determined using any of the methods described herein. The planned radiation fluence may be adjusted to the target anchor location and/or the target anchor location may be used to calculate a radiation fluence for delivery.

In the case of treatment planning for biology-guided radiotherapy, the firing filters may be calculated based on an image of the anchor location (or an image of an anchor function derived from the anchor location), and/or an uncertainty model of the location estimator algorithm(s), instead of PET images. That is, instead of generating firing filters by optimizing for a prescribed radiation dose as associated with PET imaging data, firing filters may be generated by optimizing for a prescribed radiation dose associated with an image of the target anchor location and/or a location uncertainty model (where optimization includes the process of minimizing at least one cost function and/or constraint on dose to attain the prescribed radiation dose). In some variations, an adaptive correlation filter may be generated based on a target region contour and anchor defined using planning CT images and planning PET images (or any biologically-related imaging data) aligned to the planning CT images. Planning PET images may be aligned to the planning CT images by, for example, aligning the high-intensity regions of the PET image (or any region of the PET image that is likely to be the target region and/or anchor point) with the target anchor location on the planning CT images.

FIG. 5C depicts a flowchart of one variation of a method 520 for generating an ACF h based on planning CT images and planning PET images fᵢ. While the method 520 is described as being performed during biology-guided radiotherapy treatment planning (i.e., before a biology-guided radiotherapy treatment session), in some variations, method 520 may be performed during a biology-guided radiotherapy treatment session, for example, as part of online or real-time treatment adaptation. In some variations, the adaptive correlation filter may be generated using an initial set of PET imaging data acquired just prior to the biology-guided radiotherapy treatment session (e.g., PET prescan). Method 520 includes obtaining 521 the CT imaging data and a set of PET images fᵢ of a target region, selecting 523 a target anchor location based on a contour of the target region, aligning 525 the PET images with respect to the contour of the target region, defining 527 an image of an anchor function centered on the anchor location, and generating 529 an adaptive correlation filter using the PET images and the image of the anchor function. In some variations, the obtained CT imaging data may be used to generate one or more CT images, and the target region contour may be defined on the one or more CT images. The CT imaging data may be acquired over a plurality of patient platform positions and in some examples, may be obtained using a diagnostic CT imaging system. The set of PET images may optionally be obtained using a diagnostic PET imaging system and/or the PET detectors on a biology-guided radiotherapy PET imaging system. The PET images may be reduced-motion PET images (e.g., “motion frozen” or “deblurred” PET images where motion artifacts are removed or suppressed). The contour of the target region may be defined by a clinician, with or without the aid of contouring tools and algorithms (e.g., auto-contouring software). In some variations, the contour of the target region may be a planning target volume (PTV) contour, an internal target volume (ITV) contour, a biology-tracking zone (BTZ) contour, a clinical target volume (CTV) contour, a gross tumor volume (GTV) contour, and/or the contour or boundary of a PET-avid region.

Aligning 525 the PET imaging data to the target contour, which may be, in some variations, aligning a central portion of the PET imaging data over the selected target anchor location. In some variations, aligning 525 the PET images with respect to the target region contours (on CT images, for example) may include registering the images such that corresponding regions in space are situated in corresponding locations in the image, which may or may not result in concentric alignment of the target contour and the PET imaging data. For instance, a selected landmark (e.g., anatomical structure) in the CT image may be aligned with the corresponding landmark in the PET image. In some variations, a region of the PET image having a cluster of high-intensity pixels (e.g., pixels having an intensity value above a threshold value) may be aligned with the target region contour in the CT image, as it is likely that a PET-avid region is a tumor which is to be irradiated.

In some variations, the adaptive correlation filter that is generated during treatment planning may be updated (e.g., trained, adapted) with additional imaging data. In the case of biology-guided radiotherapy, the adaptive correlation filter may be updated using PET prescan imaging data acquired at the beginning of a treatment session. The adaptive correlation filter may be updated or trained using reduced-motion PET images (e.g., deblurred or motion-frozen images). In some variations, an adaptive correlation filter may be tested using datasets comprising imaging data and corresponding target region location data (e.g., “ground truth” location of the target region). The adaptive correlation filter may be applied to the imaging data to generate an estimated target region location, and the estimated target region location may be compared with the “ground truth” location of the target region. If the estimated target region location generated using the adaptive correlation filter differs from the “ground truth” location (e.g., difference is outside an expected or acceptable range), the adaptive correlation filter may be trained/updated further on additional imaging and location data of the target region. Optionally, the cumulative PET imaging data as part of biology-guided radiation delivery may be used to generate a treatment time PET image (i.e., PET image generated at the time of treatment) and the treatment time PET image may be used to further update the adaptive correlation filter (e.g., for the next treatment session). In some variations, the quality of the treatment time PET image may be evaluated before it is used to update the adaptive correlation filter. That is, in order to use a treatment time PET image to update the adaptive correlation filter, the regions of the PET image having the highest intensity levels may need to have substantial overlap and/or alignment with the target region contour on a planning and/or localization CT. Alternatively, or additionally, a confidence metric may be used to evaluate whether an image (e.g., filtered image, cross-correlation image) may be used to update the adaptive correlation filter. For example, a confidence metric value of an image may be calculated and if its value is above a threshold value, the image may be used to update the adaptive correlation filter. Alternatively, or additionally, an error metric may be used to evaluate whether an image (e.g., filtered image, cross-correlation image) may be used to update the adaptive correlation filter. For example, an error metric value of an image may be calculated and if its value is below a threshold value, the image may be used to update the adaptive correlation filter.

FIG. 5D depicts one variation of a method 530 for updating the adaptive correlation filter ACF h based on PET imaging data acquired during a biology-guided radiotherapy treatment session. The method 530 includes obtaining 532 new PET imaging data, aligning 534 the new PET imaging data with respect to a planned anchor location and/or contour of the target region in a CT image, and updating 536 the ACF h using a new set of PET imaging data. The new PET imaging data may be acquired over various patient platform positions. In some cases, the new PET imaging data may be obtained during a prescan procedure (e.g., the PET images may be obtained before starting the biology-guided radiotherapy treatment session). In some cases, the new PET imaging data may be obtained using a diagnostic PET imaging system.

In some variations, aligning 534 a PET image with respect to the target region contours (on CT images, for example) may include selecting a landmark (e.g., anatomical structure) in the PET image and aligning the selected landmark with the corresponding landmark in the CT image. Various alignment approaches, as previously described in relation to step 525 of method 520 may be used for the aligning step 534.

In some variations the updating 536 of the ACF h may include updating H* (herein, as before, H*=[FT(h)]*). The H* may be updated iteratively based on an additional PET image fᵢ. For example, if

$${H}_{i - 1}^{*}$$

is a complex Conjugate of a Fourier transform of the ACF h before the update, the complex conjugate of the updated Fourier transform is denoted as

$${H}_{i}^{*}$$

and may be given by

$${H}_{i}^{*} = {A}_{i} / {B}_{i} , {A}_{i} = η {G}_{i} ⊙ {F}_{i}^{*} + ( 1 - η ) {A}_{i - 1} , \\ and {B}_{i} = η {F}_{i} ⊙ {F}_{i}^{*} + ( 1 - η ) {B}_{i - 1} , \\ and {A}_{0} = {\sum }_{1}^{{k}_{m}} {G}_{k} ⊙ {F}_{k}^{*} , {B}_{0} = {\sum }_{1}^{{k}_{m}} {F}_{k} ⊙ {F}_{k}^{*} .$$

Herein, as described above, Gᵢ=G_{i,uv} and Fᵢ=F_{i,uv} are two dimensional matrices (for two dimensional images), and operator ⊙ implies elementwise multiplication

$${G}_{i} ⊙ {F}_{i}^{*} = {G}_{i , uv} \cdot {F}_{i , uv}^{*} .$$

It should be noted that in some cases, Gᵢ and Fᵢ may be three-dimensional images matrices Gᵢ=G_{i,uvw} and F_{i,uvw} corresponding to a volumetric representation of the PET image fᵢ and operator ⊙ implies elementwise multiplication

$${G}_{i} ⊙ {F}_{i}^{*} = {G}_{i , uvw} \cdot {F}_{i , uvw}^{*} .$$

Herein, and elsewhere, as described before Gᵢ=FT(gᵢ), and Fᵢ=FT(fᵢ). Further, parameter n describes contribution (e.g., weight) of the new PET image fᵢ when updating Hᵢ. For example, when parameter η=0, no update is performed, and when parameter η=1, only new PET image fᵢ is considered when calculating updated Hᵢ. In general n may be a relatively small number (e.g., n may be in the range of about 0.01 to about 0.2). In some cases, η~0.1. Once Hᵢ is computed, the updated ACF hᵢ can be computed as hᵢ=FT⁻¹(Hᵢ). Further, note that images indexed k=1 to kₘ (e.g., images represented by Fₖ) and functions Gₖ correspond respectively to a training set of images and delta functions for generating an initial estimate for the ACF h (herein, kₘ is the number of images).

While some methods for updating or training an adaptive correlation filter (e.g., Hᵢ) may include calculations that rely on the previously-calculated adaptive correlation filter (e.g., Hᵢ₋₁), other variations for updating/training an adaptive correlation filter may use the initial adaptive correlation filter (e.g., H₀), and not require the previously-calculated adaptive correlation filter. In other words, some methods for updating or training an adaptive correlation filter do not comprise iterative training steps, but instead update or train the adaptive correlation using the current set of PET imaging data (e.g., stitched PET images). Instead of calculating an updated ACF

$${H}_{i}^{*}$$

using a previous value of ACF

$${H}_{i - 1}^{*} ,$$

the updated ACF

$${H}_{i}^{*}$$

may be calculated directly from the initial ACF

$${H}_{0}^{*}$$

and the newly acquired imaging data P and the initial anchor function G. For example, new and/or updated PET image p and its Fourier transform P may be denoted as:

$$p \approx {\sum }_{i = 1}^{N} {f}_{i}$$

$$P \approx {\sum }_{i = 1}^{N} {F}_{i}$$

With this, updating an ACF using N additional PET imaging data to an updated ACF H_{N} need not include previous values of the ACF Hᵢ₋₁, but may instead be calculated from the initial ACF H (which is given by

$${H}_{0} = \frac{{A}_{0}}{{B}_{0}} )$$

and the additional PET imaging data, as expressed below:

$${H}_{N} = \frac{{A}_{N}}{{B}_{N}} = \frac{{A}_{0} + \frac{1}{N} G * P}{{B}_{0} + \frac{1}{N} P * P}$$

Where H_{N} is the updated ACF that has been trained on N additional PET images, P is the Fourier transform of a PET image (e.g., a PET image stitched together from multiple partial PET images), A₀, B₀, are initial components or templates of the initial ACF H₀, and G is the Fourier transform of the initial anchor function g, * is the component-wise product.

Some variations of methods for training and/or updating an adaptive correlation filter may comprise using deblurred images (i.e., reduced-motion images) where the target location in each image is aligned to the same position. In this variation, a constant anchor function g (i.e. an anchor function at a single location or position) may be used to train and/or update the ACF. Conceptually, this method trains the ACF (e.g., templates for the ACF) using images of the target at a single position; that is, the ACF is trained using static, deblurred images and a static anchor function (e.g., at zero shift). Alternatively, or additionally, a variation of a method for training and/or updating an adaptive correlation filter may comprise using non-static, non-deblurred images and a dynamic anchor function comprising a positional shift of the target. The positional shift may be determined using deblurred images of the target, using any of the methods described herein. The positional shift may be applied to the baseline or constant anchor function g to attain a dynamic anchor function gᵢ. Each image used for training may be paired with its corresponding dynamic anchor function gᵢ which encodes the target shift identified by deblurring the image of the target. That is, each non-deblurred LTS image fᵢ is associated with a dynamic anchor function gᵢ.

For example, a method for updating and/or training an ACF based on LTS images may use the original (i.e., not deblurred) LTS images and a dynamic anchor function comprising a target positional shift. The target positional shift may be empirically measured and/or calculated using deblurred LTS images. The method may comprise acquiring and/or loading LTS images of a target at an initial position and while it is moving along a motion trajectory, where the LTS images are non-deblurred LTS images (e.g., may contain motion artifacts), calculating a positional shift of the target from the initial position for each section of the motion trajectory, shifting an anchor function for each section of the motion trajectory by the corresponding positional shift, and generating an adaptive correlation filter

$${H}_{N} = \frac{{A}_{N}}{{B}_{N}}$$

using the non-deblurred LTS images and the shifted anchor function for each section of the motion trajectory. The positional shift may be calculated using any of the methods described herein, and/or may be empirically measured using a position sensor placed on the patient or phantom while acquiring the imaging data.

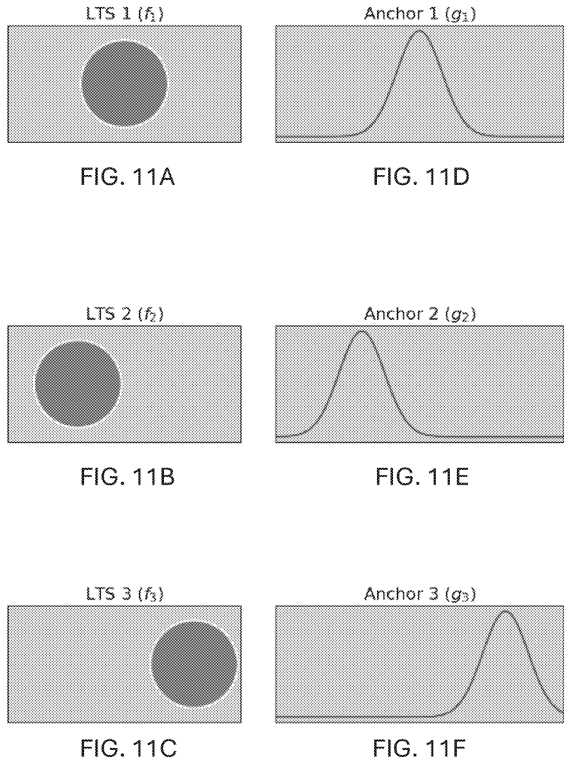

FIGS. 11A-11F conceptually depict the training data that may be used to update an adaptive correlation filter. Each training data may comprise a non-static, non-deblurred image (fᵢ) such as a LTS image and a dynamic anchor function (gᵢ) that is derived from a baseline or constant anchor function that has been shifted by the shift corresponding to that LTS image. For example, FIG. 11A depicts an “original” image LTS 1 (g₁) and which corresponds with Anchor 1 (g₁) with its associated position shift (i.e., the anchor function is labeled with the shift determined using any of the methods described herein), shown in FIG. 11D. The second training data may comprise an “original” image LTS 2 (g₂) depicted in FIG. 11B and which corresponds with Anchor 2 (g₂) with its associated position shift, shown in FIG. 11E. The third training data may comprise an “original” image LTS 3 (g₃) depicted in FIG. 11C and which corresponds with Anchor3 (g₃) with its associated position shift, shown in FIG. 11F. Without wishing to be bound by theory, this method may avoid updating or training an adaptive correlation filter on images that may have certain image processing artifacts, such as circshift artifacts caused by wrapping one end of the volume to the other. For example, some variations of deblurring a plurality (e.g., collection) of LTS images comprise applying a circshift to each LTS image (e.g., shifting the pixels of the image circularly, meaning pixels that shift off one end are re-inserted at the beginning). Applying a circshift to an image may introduce artifacts caused by wrapping one end of the volume to the other. Such circshift artifacts may bias the template(s) of the ACF. Because the method described above does not train and/or update the ACF using deblurred images that may have circshift artifacts, the resultant template and/or ACF will not be affected by such artifacts. More generally, updating or training an adaptive correlation filter on “raw” or “original” images may help tune the filter to more precisely identify the target region in its surrounding environment, which may vary depending on the location of the tumor region along the motion trajectory. This may help the adaptive correlation filter be more robust when processing imaging data that may have greater variability in background noise, artifacts, and target region geometries (e.g., the perceived size and shape of a tumor region could vary along its motion trajectory).

### Template Dot Product Location Estimation

Another algorithm or method for estimating the location of a target region in a PET image may comprise creating a plurality of image templates of the target region that each represent a different location (or shift from a nominal or center position) of the target region within the BTZ, calculating a dot product of each image template with the PET image, and identifying the target region location that corresponds with the image template that results in the highest dot product. In some variations, the PET image may comprise a plurality of partial PET images acquired during short acquisition time windows (e.g., limited time-sampled images or LTS images), and calculating the dot product may comprise calculating the dot product of each image template with a partial PET image to identify the target region location (or shift) within the partial PET image. In some variations, the target region shift (or location) may be calculated using Equation 3:

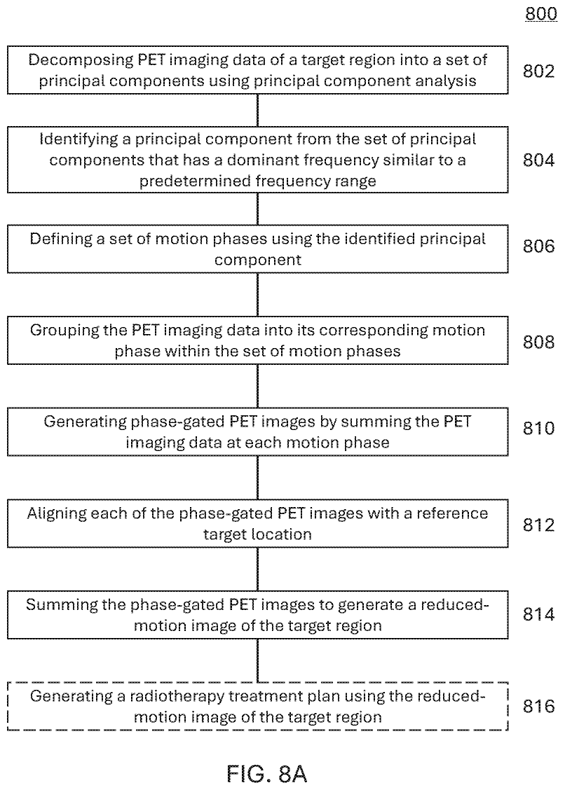

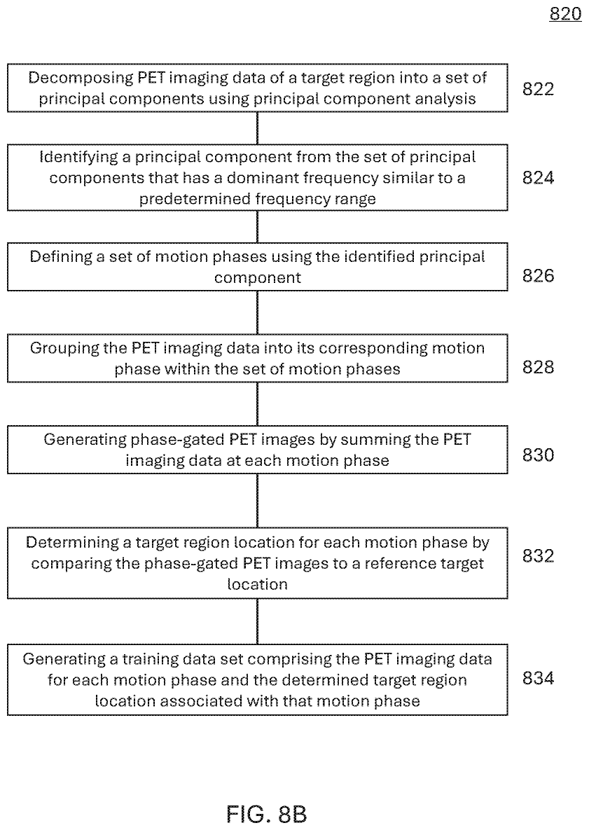

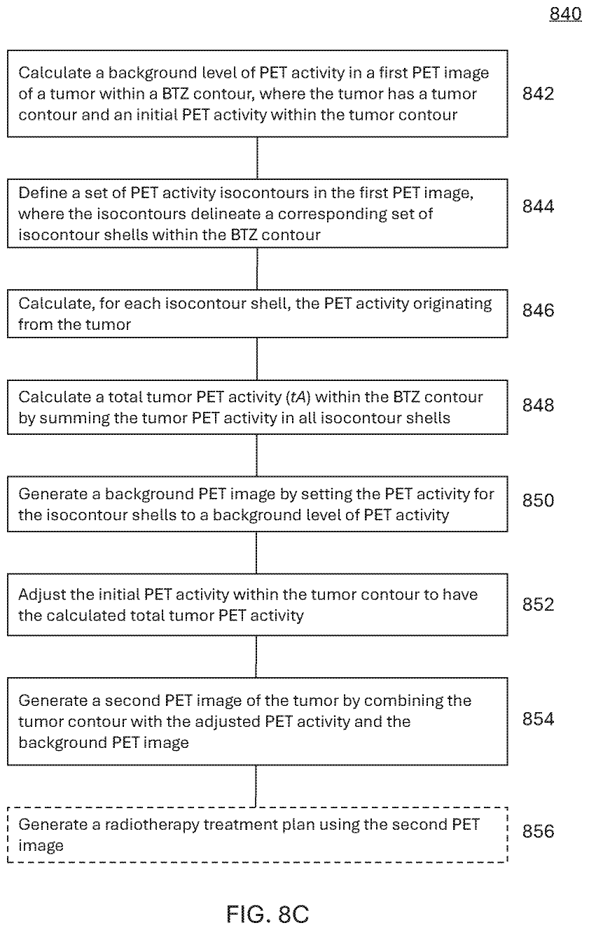

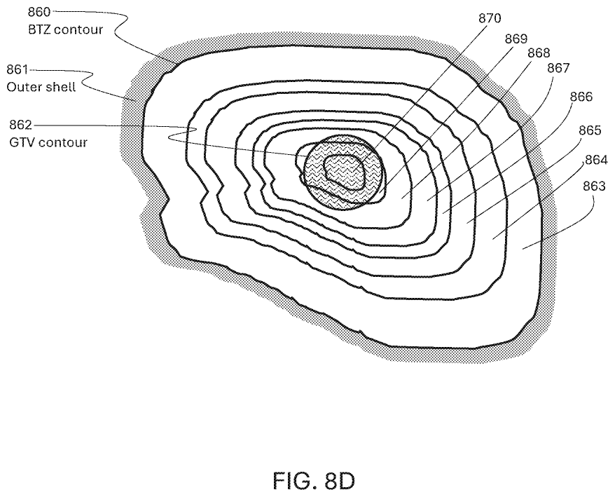

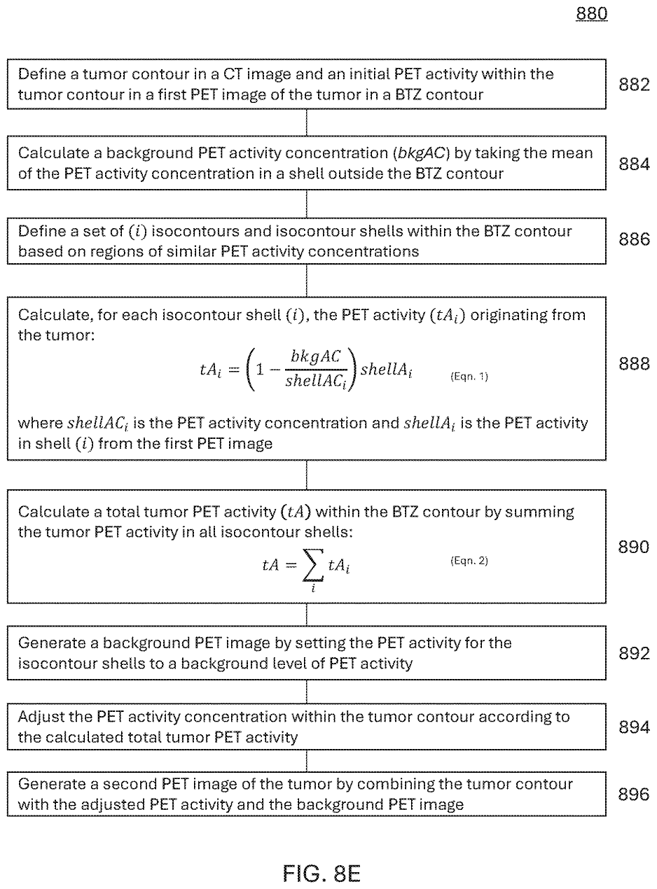

θₑₛₜ [symbol] argmax_{θ}(Σᵢnᵢp_{i,θ}) (Equation 3)

where θₑₛₜ is the estimated target region shift (or estimated target location), θ is a set of possible target region shifts (or possible target region locations) inside the BTZ, p_{θ} is a set of image templates each corresponding to a certain shift and/or location of the target region, n is the acquired imaging data (e.g., a LTS image, partial PET image) and i is the image voxel index. In some variations, a ramp filter may be applied to PET images for generating one or more template images and/or to process the acquired imaging data. Alternatively, or additionally, the image templates may be generated from deblurred or reduced-motion PET images, which may be derived using the methods described below (e.g., in FIGS. 8A-8E), and/or any other suitable method. For example, template images may be generated by deblurring PET images of a target region to attain a contour of the target region, generating a first template image corresponding to a first shift or location by shifting or moving the target region contour to a first location within the BTZ, generating a second template image corresponding to a second shift or location by shifting or moving the target region contour to a second location within the BTZ, and repeatedly shifting or moving the target region contour to a desired number of locations to generate the desired number of template images.

### Kalman Filter

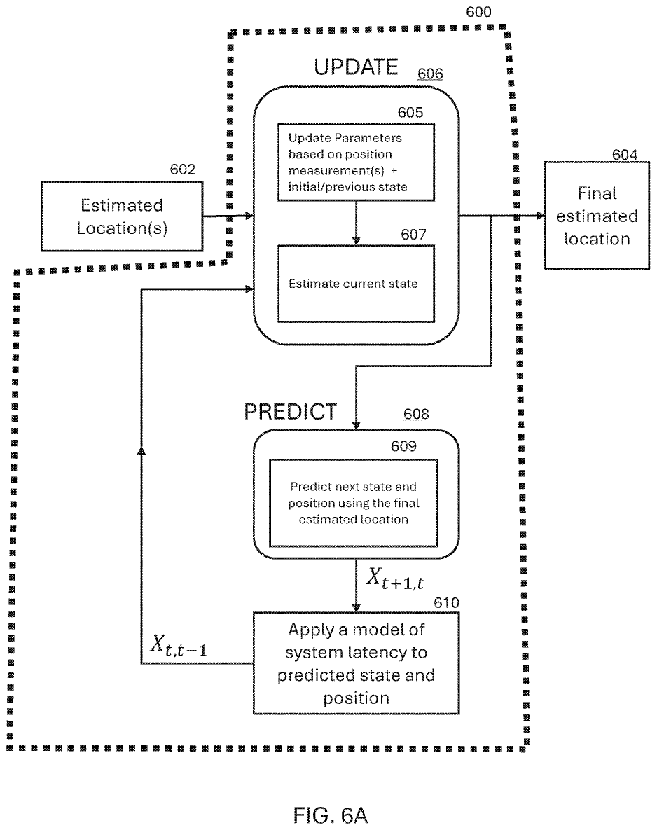

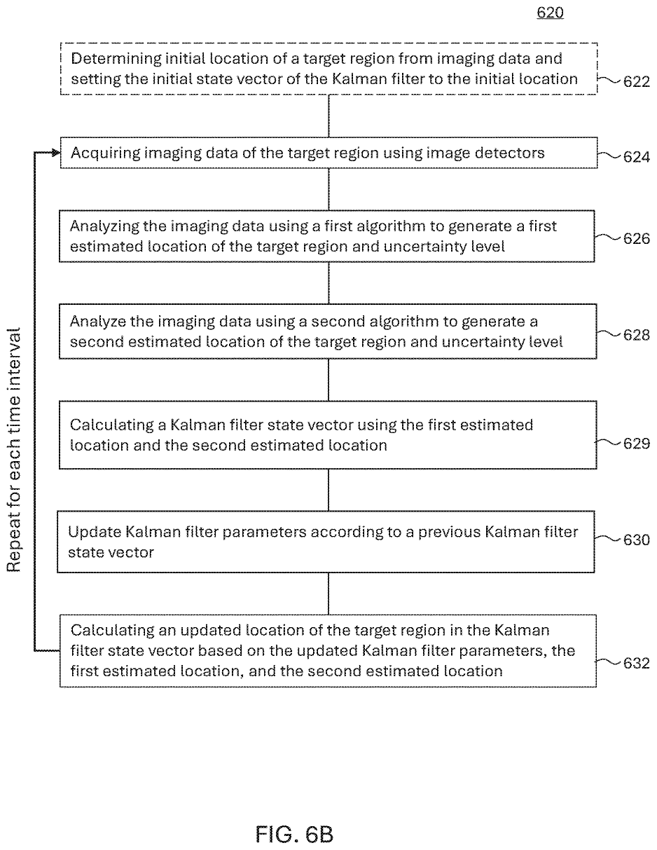

Kalman filtering is a linear quadratic estimation method that generates an estimate of an unknown variable based on a series of measurements of that variable. The measurements may have varying levels of noise and/or inaccuracies, but when combined using a Kalman filter, the final estimate of the variable may be more accurate than any one of those measurements. The Kalman filter may be considered an optimal estimator that intelligently fuses these multiple data streams. It may weigh each location estimate based on its calculated uncertainty (confidence) metric or model. It may also use a velocity and acceleration model of the target region's motion to produce a final estimate that is smoother, more accurate, and more physiologically plausible than either of the individual location estimates. FIG. 6A is a conceptual block diagram of one variation of an implementation of a Kalman filter that may be used to generate an estimated location of a target region by combining one or more location estimates from other location estimation methods or algorithms (e.g., any one or more of the methods described herein). A Kalman filter 600 may comprise a plurality of parameters whose values may be initially set by a user, and then updated as the Kalman filter is applied to additional data. In one variation, a Kalman filter may comprise parameters including a state vector (x), a state transition matrix (F), an observation matrix (H), a process noise covariance matrix (Q), and a measurement noise covariance matrix (R). The Kalman filter state vector (x) comprises the target region position, velocity, and acceleration. These values may be represented in 3D coordinates, such that the state vector (x) has one element for each of x, y, z values for position, x, y, z, values for velocity, and x, y, z, values for acceleration. The state transition matrix (F) may comprise kinematics equations that model motion properties of the target region. Observation matrix (H) may contain a mapping that indicates the relationship of the Kalman filter state vector to the input measurements (in this example, the input measurements are the estimated locations calculated from a plurality of location estimation methods or algorithms. The process noise covariance (Q) includes physical noise values in the system and the measurement noise covariance matrix (R) includes measurement noise values. For example, each location estimation method or algorithm may generate an estimated location along with a corresponding confidence metric and/or error metric that represents the accuracy and/or precision of the estimation method or algorithm, and the measurement noise covariance matrix (R) may be updated to include the confidence metric and/or error metric for each estimated location. Locations generated by a method with a high degree of confidence and/or low likelihood of errors may be given more weight in the calculation of the final estimated location than locations generated by a method with a low degree of confidence and/or high likelihood of errors. Estimated location(s) 602 may be inputs to the Kalman filter 600 and may be used, along with the previous values of the filter parameters (if available), to update one or more of the filter parameters. The updated Kalman filter parameters which represent a combination of the estimated location(s) 602 of the target region may be used to calculate a final estimated location 604 of the target region. FIG. 6A depicts one variation of a Kalman filter 600 that may combine estimated locations 602 to generate a final estimated location 604 of the target region by updating 606 the filter parameters, predicting 608 the next values of the Kalman filter state, modeling 610 imaging system latencies in the predicted Kalman filter state, and which may then be used with the next set of estimated locations 602 to update 605 the filter parameters. Updating 606 the filter parameters may comprise updating 605 parameters based on the input position measurement(s) and initial/previous state, and estimating 607 the current state of the Kalman filter. For the first iteration, the state (i.e., state vector values) may be assigned to an initial condition of the target region, i.e., initial position, initial velocity, initial acceleration), but for successive iterations, the state may be calculated from a previous state or iteration of the Kalman filter 600. Updating 605 parameters of the Kalman filter may comprise updating the observation matrix (H) and measurement noise covariance matrix (R) according to the input estimated locations 602 and their corresponding confidence metrics and/or error metrics and/or uncertainty metrics. The position values of the estimated current state 607 may be output as the coordinates of the final estimated location 604.

Estimating 607 the current state of the Kalman filter may comprise calculating the values of the state vector elements using the updated observation matrix (H) and measurement noise covariance matrix (R). In some variations, the measurement noise covariance matrix (R) may be a static, hard-coded sensor uncertainty. Alternatively, or additionally, the measurement noise covariance matrix (R) may comprise a dynamic uncertainty that may be calculated during treatment planning/prescan/delivery (e.g., using the confidence metrics and/or error metrics and/or uncertainty metrics derived from training the ACF and/or MLE methods) and/or updated 605 according to the input estimated locations and their corresponding confidence metrics and/or error metrics and/or uncertainty metrics. For example, one method for computing a dynamic uncertainty may comprise applying one or more location estimation methods, such as the ACF location estimation algorithm and/or the MLE location estimation algorithm, to imaging data that has been deblurred (i.e., processed to have motion artifacts removed or reduced, resulting a reduced-motion image) to determine a target region shift, and computing a location uncertainty quantity (e.g., uncertainty cloud, error bar, confidence metric, etc.) from the determined target region shifts over multiple sets of imaging data. This location uncertainty may be used as an input to the Kalman filter, for example, during initialization of the Kalman filter. The estimated current state 607 may also be used together with the coordinates of the final estimated location to predict 609 the next state (including the position elements X_{t+1,t} of the state vector). A model of imaging system latencies (e.g., from signal transmission times, detector imperfections, timing constraints, etc.) may be applied 610 to the predicted next state of the Kalman filter. The predicted next state, including a predicted position X_{t,t-1} (which may be considered the current state of the Kalman filter for the next iteration), may be used to update 606, 605 Kalman filter parameters in conjunction with a new set of input position measurement(s).

FIG. 6B is a flowchart depiction of one variation of a method for combining a first estimated location and a second estimated location of a target region using a Kalman filter to generate a final estimated location of the target region. Method 620 may comprise acquiring 624 imaging data of the target region using image detectors, analyzing 626 the imaging data using a first algorithm to generate a first estimated location of the target region and first uncertainty level/metric, analyzing 628 the imaging data using a second algorithm to generate a second estimated location of the target region and second uncertainty level/metric, calculating 629 a Kalman filter state vector using the first estimated location and the second estimated location, updating 630 Kalman filter parameters according to a previous Kalman filter state vector, and calculating 632 an updated location of the target region in the Kalman filter state vector based on the updated Kalman filter parameters, the first estimated location, and the second estimated location. The updated location of the target region and/or the Kalman filter state vector may be used in a following iteration or application of the Kalman filter, as a “previous” state. Optionally, some variations of method 620 may comprise determining 622 an initial location of the target region from imaging data and setting the initial state vector of the Kalman filter to the initial location. In some variations, the imaging data may comprise PET imaging data acquired during a planning imaging scan and/or a PET prescan just prior to radiotherapy delivery. The imaging data may be acquired 624 using PET detectors and may be PET imaging data such as LOR data, listmode data, time-of-flight PET data, and any positron annihilation event data. The first and second location estimation algorithms may be any of the algorithms or methods described herein, e.g., maximum likelihood estimation methods, adaptive correlation filter methods, template matching methods, and/or methods that use time-of-flight PET data for calculating the centroid of a PET-avid target region. Calculating a 629 a Kalman filter state vector and/or calculating 632 an updated location of the target region in the Kalman filter state vector may optionally use confidence metric and/or error metric and/or uncertainty levels for each of the estimated locations. In some variations, the uncertainty level/metric may be determined in advance and input as a setting or factor into the Kalman filter. For example, there may be a certain level of uncertainty due to the radiotherapy system latency of about 500 ms from the time a PET LOR is determined or detected to when a radiation beamlet may be emitted in response to that LOR. This may introduce some uncertainty and/or error in the location estimation, and since the latency of about 500 ms is known, the location uncertainty and/or error in the location estimation may be deterministic. Method 620 may be repeated multiple times during a radiation delivery session. For example, method 620 may be repeated for multiple time intervals during a treatment session during the delivery of radiation. Calculating a final estimated location of the target region by combining the first and second estimated locations using a Kalman filter may be more accurate and/or precise than the estimates on their own. The final estimated location of the target region may have a higher confidence metric and/or lower error or uncertainty metric than the estimates on their own. The accurate and/or precise location of a target region in real-time during a radiation delivery session may improve the dose conformality of the delivered radiation, so that the prescribed radiation dose is delivered to the target region while limiting the irradiation of non-target tissue. Moreover, calculating the radiation fluence for delivery using the location of the target region instead directly calculating the radiation fluence from the imaging data may help limit the effect of imaging artifacts and/or noise on the quality of the radiation dose delivery.

Optionally, some methods may comprise testing the accuracy and/or precision of the Kalman filter using datasets comprising imaging data and corresponding target region location data (e.g., “ground truth” location of the target region). For example, the imaging data and corresponding target region location data may be time-of-flight PET data in conjunction with the centroid locations, and/or any simulated imaging data. The estimated locations from multiple location estimation methods may be combined using a Kalman filter as described above to generate a final estimated location. The final estimated target region location may be compared with the “ground truth” location of the target region. If the estimated target region location generated using the Kalman filter method described herein differs from the “ground truth” location (e.g., difference is outside an expected or acceptable range), the Kalman filter may be trained/updated further on additional imaging and location data of the target region. In some variations, the comparison may be used to determine a confidence metric that may reflect the accuracy and/or precision of the location estimation method. The confidence metric and/or error metric values and/or uncertainty levels from testing the location estimation methods prior to radiation delivery may also be measured during radiation delivery when the location estimation methods are applied to imaging data acquired during radiation delivery. The confidence metric and/or error metric values and/or uncertainty levels during radiation delivery may be compared to the same metrics during pre-treatment testing. If these metric values differ beyond an acceptable range, the radiotherapy system controller may generate a notification to the clinician to evaluate whether the radiation delivery should proceed.

### Treatment Planning Using Reduced-Motion Imaging Data

Typically, radiotherapy treatment planning methods for IMRT and SBRT include acquiring images of the target region, defining one or more contours around the target region(s), defining one or more contours around organs-at-risk (OARs) and/or non-target tissue, calculating a planned radiation fluence map that delivers a prescribed radiation dose to the target region(s) while limiting radiation exposure to OARs and/or non-target tissue, and generating a set of radiotherapy system instructions from the planned fluence map that, when executed by the radiotherapy system, delivers the prescribed dose to the target region(s) and limits irradiation of non-target tissue. The treatment planning images may include CT images, and optionally, MR images, PET images, ultrasound images, and the like. This imaging data may facilitate the definition of the various contours and result in the generation of a treatment plan that may result in the delivery of the prescribed dose to the target region(s), if the target region(s) remain in the position that they were in during treatment planning.

In biology-guided radiotherapy, treatment planning images may include CT images and PET imaging data, where the PET imaging data has been acquired using the PET detectors that are on-board the radiotherapy system that will be used to deliver the radiation dose. Treatment planning for biology-guided radiotherapy comprises calculating a mapping function that converts imaging data into radiation fluences. The mapping function designates the relationship between imaging data and radiation fluence and is configured to transform imaging data into a corresponding radiation fluence. In some variations, the mapping function comprises firing filters. The mapping function (or firing filters) is calculated for each target region during treatment planning based on a prescribed dose to a target region, the defined contours, and imaging data of the target region (e.g., PET imaging data, an image of a target anchor, CT imaging data, etc.), and/or a location uncertainty model, and by iteratively solving for a fluence map that minimizes at least one cost function while still providing the prescribed dose to the target region. During a biology-guided radiotherapy treatment session, the firing filters are applied (e.g., convolved) with the acquired PET imaging data to generate a corresponding radiation fluence. The radiotherapy system controller then segments (e.g., converts, translates) this radiation fluence into radiotherapy machine instructions (e.g., instructions for the MLC, linac, gantry, patient platform, etc.) so that the radiation fluence can be quickly delivered to the target region. Since tumors may change their position between planning and treatment, and/or may move during a treatment session, the real-time acquired PET imaging data may help guide the delivered radiation to the actual location of the target region(s). However, as described above, artifacts and/or noise in the real-time acquired PET imaging data may affect the quality (e.g., conformality) of the delivered radiation dose. The methods described herein may help mitigate the impact of imaging noise and/or artifacts by determining the location of the target region (e.g., location of a tumor centroid or any tumor feature or landmark), generating an image of the target region location which may have little or no imaging artifacts and/or noise (i.e., fewer than the real-time acquired imaging data), and convolving the image of the target region location with firing filters. In the variations described herein, calculating the firing filters during biology-guided radiotherapy treatment planning may use “motion-frozen” or “deblurred” PET images that have little or no motion artifacts. In some variations, calculating the firing filters may use images of the location (of the target region) in conjunction with the reduced-motion PET images. Alternatively, or additionally, calculating the firing filters may use a location uncertainty model generated for the one or more location estimator methods described herein. Calculating the firing filters using reduced-motion PET images and/or images of the target region location may help improve the conformality of the delivered radiation dose, and/or maintain the conformality of the delivered radiation dose in the presence of variabilities and/or errors of the location estimation method(s) used to determine the location of a target region. Without wishing to bound by theory, the improvement in dose conformality when calculating firing filters using reduced-motion PET images may arise because during a treatment session, the acquired PET imaging data are also reduced-motion PET images, so the conversion of imaging data to radiation fluence aligns or “matches” better with the treatment plan. In other words, the firing filters may convert the real-time PET imaging data and/or location image to the planned fluence more accurately when the firing filters are calculated based on similar imaging data.

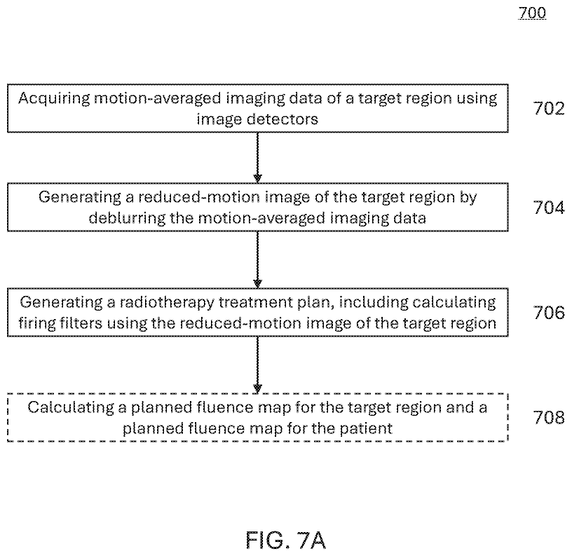

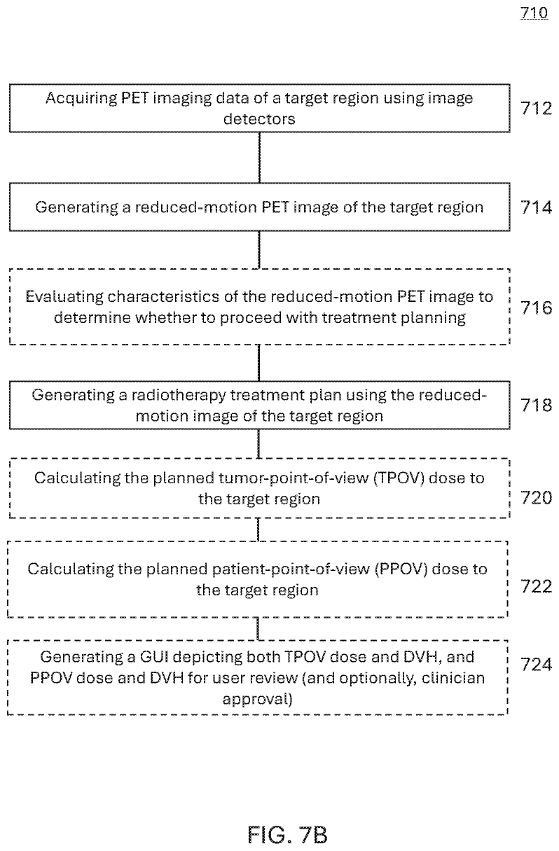

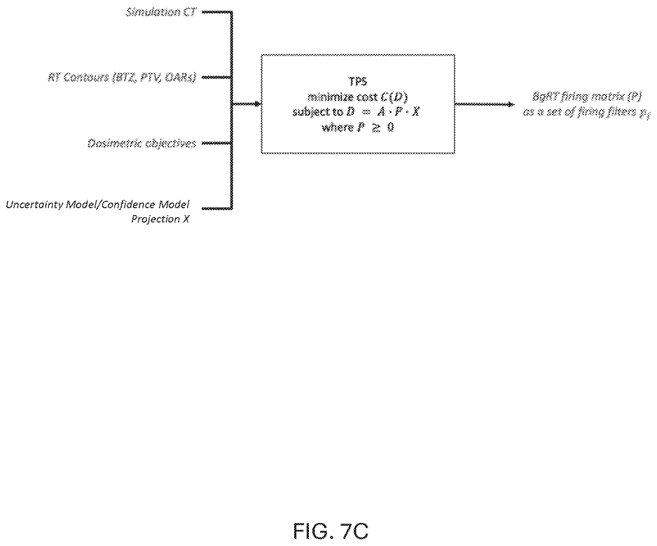

FIG. 7A depicts a flowchart representation of one variation of a treatment planning method. The method 700 may be used for any type of radiotherapy treatment planning, including SBRT, IMRT, and/or biology-guided radiotherapy. Method 700 may comprise acquiring 702 motion-averaged imaging data of a target region using image detectors, generating 704 a reduced-motion image of the target region by deblurring the motion-averaged imaging data, and generating 706 a radiotherapy treatment plan using the reduced-motion image of the target region. In some variations, the imaging data may be PET imaging data, and the motion-averaged imaging data of the target region may be acquired using PET detectors of a PET imaging system that may be a standalone imaging system or an onboard imaging system on a radiotherapy system. Generating 704 a reduced-motion image of the target region may comprise decomposing the imaging data into a set of motion phases, grouping the imaging data into the corresponding motion phase, aligning and summing the imaging data for each motion phase to attain a phase-gated PET image, and aligning and summing the phase-gated PET images over all motion phases to obtain a reduced-motion image of the target region. Variations of this method will be described in further detail below. In some variations, reduced-motion images of the target region may be available, in which case, steps 702 and 704 may be skipped and a radiotherapy treatment plan may be generated from the available reduced-motion images of the target region. Alternatively, or additionally, instead of generating a treatment plan from imaging data of the target region, the treatment planning system may generate a treatment plan using an image of the target region location, with or without including one or more uncertainty factors representing the variability of detecting a target region location. For example, an image of a tumor location (e.g., a target region anchor) may comprise a Gaussian function centered over the location of the tumor convolved with an uncertainty histogram (e.g., an error bar) that may represent the variation of a tumor location estimation algorithm or method, variability of measurement equipment and/or imaging systems, variability of image data acquisition and/or processing time, and other such systematic variations that may include imprecisions, errors, and/or timing latencies. Alternatively, or additionally, a target region anchor may comprise a Gaussian distribution (or any desired distribution) centered over the location of the tumor, without including an additional uncertainty model (e.g., uncertainty cloud).

Generating 706 a radiotherapy treatment plan may comprise calculating firing filters and a planned fluence map derived from the reduced-motion image of the target region. The firing filters and planned fluence map may optionally be derived from images of the location(s) of the target region over time. Methods of calculating firing filters and/or planned fluence map(s) are described below and additional details are provided in PCT Application No. PCT/US2023/079652 filed Nov. 14, 2023, U.S. Pat. No. 10,688,320 filed May 30, 2018, and PCT Application No. PCT/US2021/044405, filed Aug. 3, 2021, which are hereby incorporated by reference in their entirety. Alternatively, or additionally, instead of generating a treatment plan using a motion-reduced image of the target region, some variations may comprise generating a radiotherapy treatment plan using an image of the tumor location (e.g., an image of the target region location may comprise a Gaussian or delta function centered over the location of the target region), which may be referred to as a target region anchor or location function. Some variations of treatment planning may comprise calculating a planned fluence map and/or firing filters using the target region anchor. The target region anchor may comprise a distribution/function (e.g., a delta distribution/function, a Gaussian distribution/function, etc.) centered on the tumor location. For example, the target region anchor or location function may be a point, delta function, Gaussian function or the like in 3D coordinates superimposed onto the tumor or target region location. The spread of the distribution may be 1 or more voxels around the tumor location, with the location of the peak of the distribution aligned with the tumor location.

In some variations, treatment planning may comprise calculating a planned fluence map and/or firing filters using a location uncertainty model associated with the variability of detecting the location of the target region anchor. For example, an uncertainty model (e.g., error histogram, an error bar, uncertainty cloud) may represent the variation of a tumor location estimation algorithm or method an image of a tumor location. In some variations, these location variations and/or errors and/or imprecisions may be cumulatively encapsulated in the location uncertainty model, which may comprise a location uncertainty cloud or error bar. The uncertainty cloud or error bar/histogram may each have a certain spread and average uncertainty/error value. For example, an uncertainty model (e.g., error bar) may be derived from multiple sources of uncertainty (e.g., error, variability, etc.), and may include information that encompasses various sources of uncertainty (e.g., errors, variabilities), including but not limited to measurement noise, modeling error, system limitations or latencies, and/or incomplete information. In some variations, the location uncertainty model may be at least partially derived from the performance and/or confidence levels/metrics of the location estimation methods after such methods have been trained and/or updated on tumor imaging data. The uncertainty model may comprise an uncertainty cloud or error bar having a certain size or spread and/or an average value. Treatment planning may comprise calculating a planned fluence map and/or firing filters based on the convolution of the target region anchor with the uncertainty model. Alternatively, or additionally, treatment planning may comprise calculating a planned fluence map and/or firing filters based on the uncertainty model associated with a target region anchor. Additional details of various methods for calculating a location uncertainty cloud, location error bar or metric, and/or other quantitative indicators of the precision and confidence of an estimation of a target region location are described below.

Optionally, some variations of method 700 may comprise calculating 708 a planned fluence map for the target region and a planned fluence map for the patient. The planned fluence map for the target region may solely include the radiation fluence and/or exposure to the target region. The planned fluence map for the patient may include the radiation fluence and/or exposure to the entire patient.

The mapping function (e.g., firing filters) may be calculated based on an image of the target region location (e.g., the location of a target region anchor, the location of a tumor centroid, user or controller/processor selected location, etc.), and/or reduced-motion PET images, instead of motion-averaged PET images which may be blurred from the motion of the target region “smearing” across its motion envelope. That is, instead of generating firing filters by optimizing for a prescribed radiation dose as associated with motion-averaged PET imaging data, firing filters may be generated by optimizing for a prescribed radiation dose associated with an image of the target region location and/or a reduced-motion or “motion-frozen” (i.e., static) PET image. In some variations, the firing filters may be generated using an image of a tumor location (i.e., target region anchor) that includes the location of the tumor. Alternatively, or additionally, the firing filters may be generated using a location uncertainty model associated with determining the location of the tumor. In one variation, the firing filters may be generated based on the convolution of the target region anchor with the location uncertainty model. Without wishing to be bound by theory, including the location uncertainty model into the generation of firing filters may help the resultant firing filters/treatment plan to account for location estimation variabilities and errors. This may help ensure that the radiation delivery delivers the prescribed dose to the target region despite errors and/or variabilities that may occur during the treatment session. In the examples described herein optimization includes the process of minimizing at least one cost function or constraint on dose to attain the prescribed radiation dose. The planned fluence map is a radiation fluence map that when delivered, would provide the prescribed dose to the target region(s) and may be generated (e.g., calculated) through an iterative process using the dose prescription determined by a medical professional and a dose calculation matrix. In one variation, the dose prescription determined by the medical professional may include, for example, dose goals and objectives for a patient target region and/or OAR. Radiation dose (i.e., the amount of radiation absorbed by a subject) and radiation fluence FM (i.e., the amount of radiation emitted by a radiation source, and usually designated by radiation beams or beamlets) are related to each other (e.g., mapped to each other) by a dose calculation matrix A. That is,

D=A·FM

A dose calculation matrix represents a dose contribution from each of a plurality of radiation beamlet to each voxel of a patient target region (and/or OAR). For example, a dose calculation matrix A may be a (k×n) matrix where n may be a number of possible radiation beamlets {bi} and k may be the number of pre-selected voxels for a patient target region. An i-th column of the dose calculation matrix A (which has k elements) represents a dose contribution from a unity-weighted beamlet bi to each of the k voxels. Dose calculation matrix A may be calculated column-by-column, for example, by ray-tracing each beamlet's aperture along the path through a patient target region and calculating the contribution of a unity-weighted beamlet to each of the k voxels. A beamlet aperture may be an MLC aperture defined by a single MLC leaf opening (i.e., of a binary MLC or a 2-D MLC). Examples of algorithms for calculating a dose calculation matrix that may be used in any of the methods described herein may include Monte-Carlo simulation, collapsed-cone convolution superposition, pencil-beam convolution, and others. Each patient target region and/or OAR may have its own dose calculation matrix.

The planned radiation fluence map FM may be the radiation fluence to be emitted to the patient at each firing position in order to deliver the prescribed dose D. In one variation of treatment planning and delivery for biology-guided radiotherapy, the planned radiation fluence map FM may be represented by a firing filter p convolved with an image N of a function (e.g., a location function such as a delta function or Gaussian function, an image of a target anchor at its defined location, target region anchor, location function) centered over the selected target region location:

D=A·FM=A·(p*N)

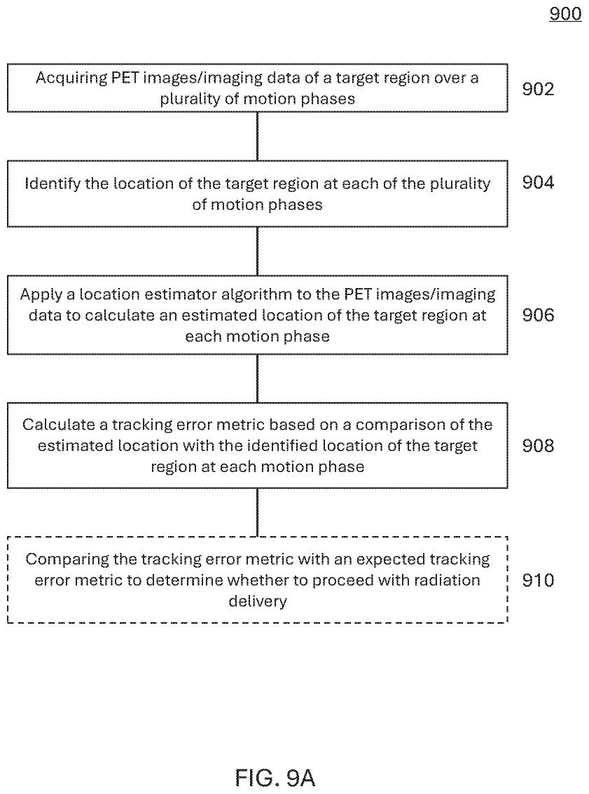

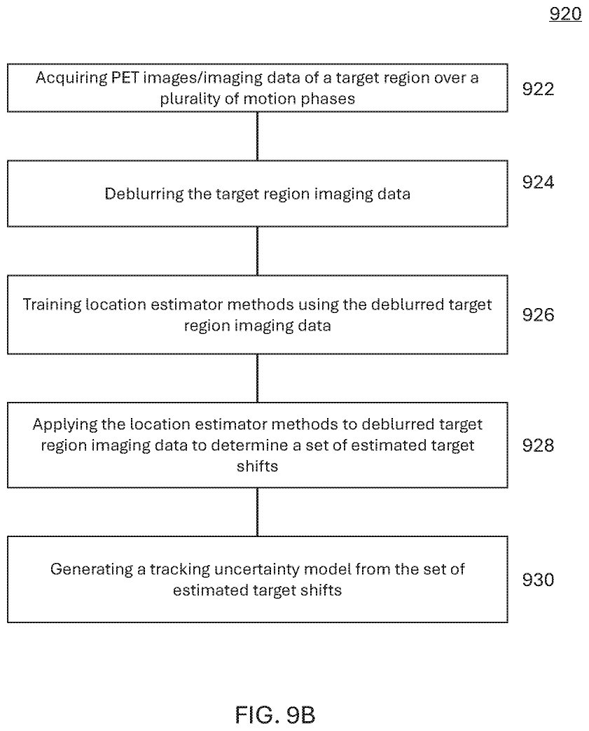

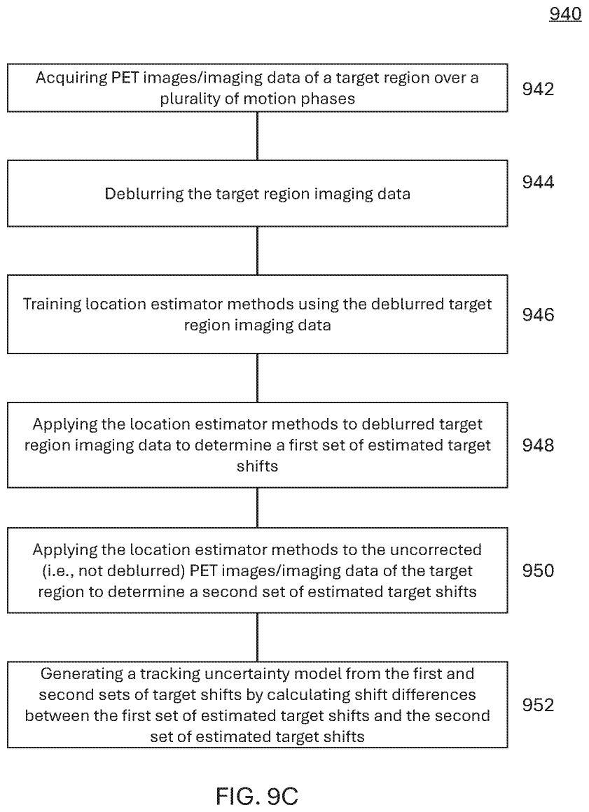

In some variations, the image N may be imaging data (e.g., PET imaging data) of the target region. Alternatively, or additionally, the image N may be a reduced-motion PET image, and/or a Gaussian or delta function centered over the location of the tumor convolved with an uncertainty histogram (e.g., an error bar) that represents the variation of a tumor location estimation algorithm or method. That is, the image N may be a target region anchor convolved with a location uncertainty model that represents the variation of a tumor location estimation algorithm or method. In some variations, the image N may be an uncertainty model that includes uncertainties and/or imprecisions of determining the exact location of the tumor using one or more location estimation methods. The uncertainty model may be, for example, an uncertainty cloud comprising likelihood values for tumor locations, with the more likely tumor locations having the higher likelihood values, and the less likely tumor locations having the lower likelihood values. As a conceptual illustration, the location of a tumor may be at (x,y,z), but the variability (e.g., uncertainty, error) of detecting its precise location may be +/−2 units, so the image N may be an uncertainty model that includes (x±2, y±2, z±2). In one variation, the image N may comprise a location function such as a delta function or Gaussian function centered on the location at (x,y,z), and an uncertainty model centered over the location function, where the uncertainty model may have a width or spread that represents the variability of determining the location. The variability (e.g., uncertainty model, error bar or metric, confidence metric) of determining the correct location of a tumor may be calculated during treatment planning and/or training of the location estimation methods. A model of the uncertainty of the one or more location estimation methods may be incorporated into the image N of the tumor, for example, as a blurred image of the tumor, a probability or likelihood distribution, probability density cloud, and/or an uncertainty cloud centered on the most-likely (or estimated) location of the tumor, and the like. Methods for calculating the variability of the location estimation methods (e.g., an uncertainty histogram or an error bar or probability density cloud) are described further below, e.g., in FIGS. 9A-9C. In addition to defining a dose prescription, a medical professional may set one or more constraints and/or cost or penalty functions C (D, FM) that specify characteristics of the dose distribution and/or radiation fluence map. Examples of cost functions may include, but are not limited to, minimum dose to target region, average or maximum dose on OARs, and/or fluence smoothness, total radiation output, total tissue dose, treatment time, etc. In one variation, a radiotherapy treatment planning system may be configured to calculate a radiation fluence map FM such that the dose prescription and constraints C (D, FM) are met. The radiotherapy treatment planning system may iterate through different radiation fluence values and/or radiation fluence maps to find the fluence values and/or fluence maps that minimize the cost function C (D, FM) while still meeting the dose prescription requirements.

To calculate firing filters p, a radiotherapy treatment planning system may set up an optimization problem for minimizing the cost function C (D, FM), given a dose calculation matrix A and an image N (which may be a function centered over the target region location, or any of the images listed above). In some variations, the anchor function in the image N used for treatment planning and/or optimization may be the same anchor function that is used to generate an image of the target anchor location (i.e., anchor point, target region anchor) during a radiation delivery session. Calculating firing filters p may comprise iterating through different firing filter values such that the cost function C (D, FM) is minimized while still attaining dose goals and objectives in accordance with the dose prescription. Firing filters may be calculated for each firing position of a therapeutic radiation source, by optimizing the dose for each firing position using projections of the images N and projections of the fluence map to each respective firing position. Generating firing filters by an iterative process based on an image of a target region location (e.g., target region anchor, location function), and/or an uncertainty model, and/or a reduced-motion PET image, and/or any of the images N described above, instead of imaging data (which may contain noise, unwanted artifacts, and/or diffuse or insufficient imaging data or photons) may help de-couple quality (e.g., conformality) of the radiation delivery from the quality of the imaging data acquired on the day of treatment. That is, calculating the location of the target region (e.g., target region anchor) and calculating the delivery fluence using an image of the target region location, instead of the newly acquired imaging data itself may help suppress or remove the effects of imaging noise on the quality of dose delivery. The image of the anchor location, which has less noise than the acquired imaging data (and in some variations, no imaging noise at all), may then be convolved with the firing filters calculated here to generate the radiation fluence for delivery.

FIG. 7C depicts a conceptual diagram of an optimization process for calculating a firing matrix comprising a set of firing filters using an uncertainty model. Optionally, prior to optimization, some methods may comprise evaluating the quality of the planning PET image deblurring method. Evaluating the quality of the deblurring method may comprise calculating a PET deblurring ratio and determining whether the deblurring ratio is within a predetermined acceptable range, calculating a spread of the uncertainty model and determining whether the spread is less than a threshold value (e.g., having units of length such as mm, cm) along any dimension, and/or calculating a mean of the uncertainty model and determining whether the mean is less than a threshold value (e.g., having units of length such as mm, cm) along any dimension. For example, the threshold value for the PET deblurring ratio may be greater than or equal to the threshold of 0.9, the threshold value for the spread (which may be defined as 2 times the standard deviation) of the location uncertainty model may be less than or equal to 20 mm along any dimension, and the threshold value of the mean of the location uncertainty model may be no more than 3 mm away (along any dimension) from the centroid of the target region. In some variations, in order to proceed with optimization, one or more of these thresholds/criteria must be met (e.g., all of these thresholds/criteria must be met). Without wishing to be bound by theory, not meeting one or more of these thresholds may indicate that the deblurring process has failed and that a clinically satisfactory and/or safe treatment plan and/or delivery is unlikely to be possible. Some methods may comprise generating an error message in the form of a GUI that is output to a display device and/or an audio notification that is output to a speaker device if one or more of these criteria are not met, where the user may then decide whether to proceed with treatment planning. Optionally, some methods may comprise halting or blocking treatment planning if any of these criteria are not met. The optimization process may be executed on a controller and/or processor of a treatment planning system. In this variation, the optimization process takes a simulation CT, one or more user-defined radiotherapy contours (BTZ, PTV, organs at risk (OARs), etc.) known as an RT structure set, dose constraints on the structures, and an uncertainty model (e.g., uncertainty cloud, confidence model) as inputs. The uncertainty model may comprise a beam's eye view projection volume (e.g., as derived from a planning PET image and/or PET images acquired on a BgRT radiotherapy system). The location uncertainty model (Iₚₗₐₙ) may be masked by the BTZ and reprojected to form a beam's eye view (BEV) uncertainty model projection volume (Xₚₗₐₙ), i.e., onto a firing position of the therapeutic radiation source. During the optimization process, instead of directly solving for a planned fluence (Fₚₗₐₙ), the methods finds the optimal linear mapping from Xₚₗₐₙ to fluence (Fₚₗₐₙ) that achieves the desired dose distribution, based on the cost function C(D) formed from user-specified dose constraints and objectives. This linear mapping may be referred to the firing matrix (P) and may comprise a set of per-firing-angle firing filters (pⱼ). These firing filters may be transferred to the delivery system (e.g., the BgRT radiotherapy system) where it may be stored in machine-readable memory. The firing filters may be later used during a radiation delivery session to calculate the machine deliverable fluence from the PET LTS images collected during the radiation delivery session.

FIG. 7B depicts a flowchart representation of one variation of a method for biology-guided radiotherapy treatment planning using motion-reduced PET imaging data. The method 710 may comprise acquiring 712 PET imaging data of a target region using PET detectors, generating 714 a reduced-motion PET image of the target region, and generating 718 a radiotherapy treatment plan using the reduced-motion image of the target region. Generating 714 a reduced-motion PET image may comprise deblurring PET imaging data using any of the methods described herein. Alternatively, or additionally, the PET images may have little or no motion artifacts, and method 710 may comprise generating the radiotherapy treatment plan from the PET images without processing or analyzing the PET images to remove or reduce motion artifacts. Generating 718 a treatment plan may comprise calculating firing filters, as described above, as well as a planned fluence map by using the reduced-motion PET image, and/or a target region anchor (which may be derived from the reduced-motion PET image), and/or a location uncertainty model, alone or in combination with the target region anchor. In some variations, the treatment plan may comprise a planned fluence map specifically for the target region and/or a planned fluence map for the entire patient. Optionally, method 710 may comprise evaluating 716 characteristics of the reduced-motion PET image (and/or the acquired 712 PET imaging data) to determine whether to proceed with treatment planning. The image characteristics may include one or more of standard uptake value (SUV) of the PET tracer by the target region, the activity concentration (AC) of the PET tracer in the target region, the PET signal contrast between the target region and background (e.g., the PET signal within the target region normalized to the PET signal outside the target region, normalized target signal or NTS), the PET signal to noise ratio for the target region (e.g., target to background ratio or TBR which may be the ratio of the activity concentration of the target region to the activity concentration of the background), and the like. Optionally, method 710 may comprise evaluating the quality of the PET deblurring process, as described herein, which may comprise calculating and evaluating the PET deblurring ratio, the spread of the uncertainty model, and/or the mean of the uncertainty model. If the image characteristics and/or the quality of the PET deblurring process do not meet predetermined thresholds for one or more of the characteristics, the treatment planning system (e.g., controller) may generate a notification to the clinician that indicates whether each characteristic met or did not meet the predetermined thresholds, and optionally, a recommendation as to whether the target region(s) are suitable for treatment. Additional details on methods for evaluating the quality of the PET deblurring process and/or evaluating the characteristics of the planning PET image (e.g., motion-corrected or motion-retained) are provided below.

Method 710 may optionally comprise calculating 720 a planned tumor-point-of-view (TPOV) dose to the target region. In one variation, the planned TPOV dose to the target region may be derived from the reduced-motion PET image and a dose volume histogram (DVH) curve. That is, the target region may be the static reference frame, and the dose calculation may be performed for the target region based on the assumption that the target region remains (i.e., is stationary) at its planned reference position. Without wishing to be bound by theory, treating the target region as stationary may help create a stable, consistent reference frame for optimization, and dose calculations for the target may be computed as if the tumor did not move relative to the planned isocenter. Frequent and precise estimations of a target location during radiation delivery may have the effect of “freezing” a moving target region as if it were stationary, i.e., delivering dose to a stationary target region. A DVH curve indicates the dose amounts (e.g., in Gy) across a proportion of the target region volume (e.g., ranging from 0 to 100%). The DVH curve may be calculated from the planned fluence map calculated using the reduced-motion PET image. Method 710 may optionally comprise calculating 722 the planned patient-point-of-view (PPOV) dose to the target region. In one variation, the planned PPOV dose to the target region may be derived from the acquired PET imaging data and a DVH curve. The DVH curve may be calculated from the planned fluence map calculated using the acquired PET imaging data that was not processed to remove the motion artifacts. Optionally, after radiation delivery (e.g., in a treatment session), the PPOV dose may be calculated using delivery parameters recorded by the radiotherapy system, for example, including gantry rotation angle, couch position, and MLC openings. Method 710 may optionally comprise generating 724 a graphical user interface (GUI) depicting the TPOV dose and corresponding DVH, and the PPOV dose and corresponding DVH. These depictions may comprise one or more of plots, diagrams, and/or color intensity maps overlaid on the acquired PET imaging data and/or the reduced-motion PET image. In some variations, the GUI may be output to a display (i.e., monitor) of a treatment planning system. This may facilitate clinician review, and optionally, approval of the generated treatment plan and associated dose distributions.

### Methods for Deblurring Images

In some variations, the images of the target region provided or acquired for treatment planning may have motion artifacts. These motion artifacts may arise from the motion of the target region during the acquisition of the imaging data by imaging detectors, and the image(s) of the target region may appear as smeared or blurred over its motion trajectory. Imaging modalities that rely on longer acquisition times for clear images may be particularly susceptible to such artifacts, since it may be difficult for a patient to remain still during the acquisition time interval and/or physiological activities (e.g., digestion, breathing, etc.) may cause involuntary shifts in the location of the target region. As described above, generating a radiotherapy treatment plan using reduced-motion or deblurred images, instead of blurry images, may result in a plan that is capable of delivering a more conformal dose to the target region. In some instances, the images of the target region may be accompanied with motion data, which may help create reduced-motion or deblurred images. For example, treatment planning images may be acquired in conjunction with a breathing monitor that measures the position of a patient's chest wall as they breathe during image acquisition. The image acquisition timing may be synchronized with the timing of the chest wall position. This data may be used by the system controller to create a set of motion phases or bins over time, and assign the imaging data to the phases or bins according to the time it was acquired. The imaging data for each phase may be summed together to obtain a clearer, less smeared, i.e., reduced-motion, or motion-corrected, image of the target region. These reduced-motion images may be overlaid or summed together across multiple phases or bins to obtain one or deblurred images that may be used for treatment planning. However, in some situations, the treatment planning images/imaging data may not have corresponding motion data, and/or may have incomplete motion data. For example, during the acquisition of the planning images/imaging data by the imaging system, the initial location and the final location of the target region may be known and stored in the system controller, while there may be little or no location data in the intervening acquisition time interval. Images or imaging data in which motion artifacts have been reduced, removed, and/or corrected may be referred to as “deblurred”, “motion-corrected,” and/or “reduced-motion” images or imaging data interchangeably throughout this description.

The methods of deblurring imaging data disclosed herein may be used in situations where imaging data of a target region is acquired over an acquisition time interval where there may be incomplete data about the motion and/or location of the target region. In one variation, generating a reduced-motion image of the target region may comprise decomposing the imaging data into a set of motion phases or bins, grouping the imaging data into the corresponding motion phase, aligning and summing the imaging data for each motion phase to attain a phase-gated image, and aligning and summing the phase-gated images over all motion phases to obtain a reduced-motion image of the target region. The motion phases may be derived from applying principal component analysis to the imaging data. The motion phases or bins may be defined or specified as time intervals, where images acquired during that time interval are grouped together with a common time stamp and/or time interval. For example, a motion phase may be specified by a time in which the phase starts and a duration, where the duration is the same for all motion phases. Alternatively, or additionally, a motion phase may be specified by two timestamps that indicate the beginning and the end of the range, where images acquired within that range are grouped or binned together. In either case, specific motion phases are defined by one or more image acquisition time stamp information or time data. Image data acquired during a specific motion phase is “assigned to” and/or “tagged with”, and/or “binned in”, and/or otherwise associated with that motion phase. In some variations, the reduced-motion image of the target region may be used to derive radiotherapy treatment plans that may have better conformality to the target region than treatment plans derived from images having motion artifacts. Alternatively, or additionally, the phase-gated images, in conjunction with location information of the target region at each motion phase, may comprise a dataset that may be used for testing and/or training any of the location estimation methods or algorithms. The location information may include shift information that indicates how much the target region shifted from a reference location (e.g., a series of shift vectors from the reference location and/or from a previously known or calculated location), and/or may include the coordinates of the actual location of the target region. For example, the dataset may comprise the location of the target region at each motion phase (which may include time stamp information and/or time data as described above) and the imaging data acquired during that motion phase. In some variations, the dataset may be a list comprising one or more of target region locations and/or shifts, the motion phases associated with each location and/or shift, and/or the imaging data acquired during each motion phase. The imaging data may be motion-corrected or may not be motion-corrected. This dataset may be used for generating one or more location estimation quality metrics, such as a location uncertainty model and/or tracking error metric, as described further below. The location and/or shift of the target region at each motion phase may be considered the “ground truth” location, and may be compared with the estimated locations calculated by one or more location estimation methods. The differences between the estimated locations and the “ground truth” location may have the purpose of testing, evaluating, and/or characterizing the precision of the location estimation method(s). The location estimation methods may be used to analyze an imaging data set to identify the location of the target region at a particular motion phase. The location identified by the location estimation method may then be compared to the “ground truth” location (e.g., as determined by the deblurring process) to evaluate the accuracy of the location estimation method. The difference between the identified location and the “ground truth” location may be used to calculate an error metric (e.g., an error bar), confidence metric, and/or uncertainty model of the location estimate method (collectively, location estimation quality metrics). The value of one or more of these metrics may indicate the quality of the location estimation provided by the method or algorithm. A clinician may view these metrics to determine whether to rely on the location estimate generated by the location estimation method for radiation delivery. In some variations, if the location estimation quality metrics indicate that the location estimation method is not sufficiently accurate or precise, the dataset used to test it may also be used to train the models, filters, etc. of the location estimation methods. Optionally, the location estimation methods may be re-tested using additional imaging data and location data of the target region.

FIG. 8A is a flowchart depiction of one variation of a deblurring method for PET images. Although the examples provided herein pertain to PET imaging data, it should be understood that these methods may also be applied to other imaging modalities, including CT imaging data, MR imaging data, optical imaging data, ultrasound data, and the like. Method 800 may comprise decomposing 802 PET imaging data of a target region into a set of principal components using principal component analysis, identifying 804 a principal component from the set of principal components that has a dominant frequency similar to a predetermined frequency range, defining 806 a set of motion phases using the identified principal component, grouping 808 the PET imaging data into its corresponding motion phase within the set of motion phases, generating 810 phase-gated PET images by summing the PET imaging data at each motion phase, aligning 812 each of the phase-gated PET images with a reference target location, and summing 814 the phase-gated PET images to generate a reduced-motion image of the target region. Method 800 may optionally further comprise generating 816 a radiotherapy treatment plan using the reduced-motion image of the target region. The PET imaging data may comprise lines-of-response data, time-of-flight PET data, listmode data, and the like, optionally reconstructed in sinogram space and/or image space. In some variations, the PET imaging data may be time-of-flight filtered back projection limited time sampled (TOF-FBP LTS) images. The PET imaging data may be acquired using PET detectors within a specified acquisition time interval, where the acquisition time interval may be about 5 seconds or less, e.g., 5 seconds or less, 4 seconds or less, 1 second or less, 500 ms or less, etc. In some variations, there may be more than one identified principal component that may be used to define the set of motion phases. For example, the set of principal components may comprise 3 or more principal components. The set of principal components may comprise a component (or components) which has the greatest PET imaging data variation. For example, the identified principal component may be a dominant principal component where its magnitude at a desired frequency (or frequency range) is greater than the magnitude of the same frequency (or frequency range) of other principal components. Identifying a principal component that has a dominant frequency similar to the predetermined frequency range may comprise applying a Fourier transform to the set of principal components. In some variations, defining the set of motion phases comprises applying a Hilbert transform to the identified principal component. This may be used to derive the instant phase for the PET imaging data acquired during that phase. The predetermined frequency range may include the frequency range of physiological processes such as breathing/respiration and/or peristaltic motion, e.g., from about 0.05 Hz to about 0.6 Hz. The set of motion phases may comprise any number of motion phases, for example, 4, 6, 8, 12, 13, 15, 16, 18, 20, or more motion phases. The reference target location may be the last known location of the target region, and/or may be the initial location of the target region determined based on imaging data acquired at the beginning of the acquisition time interval. Alternatively, or additionally, the reference target location may be an average location of the target region in the PET imaging data. For example, the PET imaging data may have a region of PET avidity that corresponds to the target region, but that region of PET avidity may be “smeared” or blurred because of target region motion. The average location of the target region may be the centroid location of the PET-avid region, for example, the average extent along the x, y, and z axes, optionally weighting the average based on target region dwell time. In some variations, method 800 may further comprise determining a phase-specific location of the target region for each phase of the set of motion phases based on the phase-gated PET images, and calculating a motion vector for each group of PET imaging data based on the phase-specific location of the target region in the set of motion phases. The phase-specific location of the target region in each phase may be used as the reference target location during alignment of the phase-gated PET images.

Aligning each of the phase-gated PET images with the reference target location may comprise determining a shift vector for each of the phase-gated PET images by comparing the phase-gated PET images with the reference target region location, and shifting each of the phase-gated PET images according to the shift vector. The shift vector may be a 3-D shift vector that provides a magnitude and direction of the shift in x, y, z space. Some variations may comprise summing the phase-gated PET images over multiple motion phases to obtain a single deblurred image of the target region using the imaging data acquired over the multiple motion phases. Determining the shift vector for each phase may comprise determining the location difference between a reference target location (e.g., the location of the highest pixel/voxel intensity for each phase-gated PET image, the location of the centroid of the PET-avid region of the image) and the location of the highest pixel/voxel intensity of the single deblurred image. The radiotherapy treatment plan may comprise a planned fluence map and/or one or more mapping functions (e.g., firing filters) that may be applied to imaging data acquired during the treatment session to convert imaging data into radiation fluence for delivery. In some variations, the shift vector and/or shift offset of a particular motion phase/bin may be associated with the PET images in that same motion phase/bin. The PET images and their corresponding shift vector/offset may be used as training data for one or more location estimation methods and/or generating a location estimation quality metric, e.g., a location uncertainty model and/or tracking error metric.

Optionally, method 800 may comprise acquiring or detecting PET imaging data of the target region using PET detectors. In some variations, method 800 may comprise applying a difference of Gaussians filter to the PET imaging data before decomposing the PET imaging data into the set of principal components.

One example of a deblurring method for PET images may comprise applying principal component analysis (PCA) to a series of PET images of a target region, selecting the first three principal components, selecting the dominant frequency of the target region motion by identifying the largest peak in the Fourier spectrum within the physiological range of 0.05 to 0.5 Hz (corresponding to about 3-30 breaths per minute), assigning a specific motion phase index assigned to each of the PET images, grouping the PET images according to their assigned phase indices, calculating a 3D shift of the images in each group by comparing the PET images in each group to a reference target location (e.g., the average location of the target region in the PET images), and applying the calculated 3D shifts to the corresponding PET images to obtain a series motion-corrected or deblurred PET images. The series of 3D shifted, motion-corrected or deblurred PET images may be combined to generate a full, motion-deblurred PET image of the target region. The PET images of the target region may be, for example, TOF-FBP LTS images (e.g., single-slice rebinned and filtered back projection LTS images) from an imaging session on the BgRT radiotherapy system. Applying PCA to these images may help to identify the most significant patterns of variation. In some variations, the first three principal components may capture the dominant motion. The range of about 0.05 to about 0.5 Hz may correspond to respiratory rates of 3 to 30 breaths per minute. In some variations, the Hilbert transform may be used to assign motion phase indices to the PET images. The motion phase index may correspond to a time interval or range in which the imaging data is grouped together. This time interval may be linked to the PET images acquired during that time interval, which may be used as training data for the one or more location estimation methods described herein. In some variations, this timestamped PET image data may be used to generate a location uncertainty model and/or calculate location error metrics, etc. Optionally, the target deblurred image may be used for aligning planning PET images with other planning images (e.g., CT images, MR images) as part of the treatment planning process. In some variations, the method may comprise calculating a 3D shift for each LTS image by comparing these phase-grouped images to the average PET position. This set of timestamped shifts may represent the 3D timestamped trajectory of the target region and may serve as a “ground truth” placeholder that may be used again later in the generating a location uncertainty model. Each of the calculated 3D shifts may be applied back to the corresponding LTS image to correct it for motion. The result is a series of motion-corrected or “deblurred” PET LTS images. Optionally, these deblurred PET LTS images may be combined to generate a full motion deblurred image of the target region.

Some variations may comprise generating a graphical representation or graphical user interface (GUI) of the deblurred image of the target region, and output the GUI to a display (e.g., monitor). The GUI may include a combination image comprising the deblurred PET image of the target region while the background of the PET image (i.e., the portion of the image that is not the target region) remains blurred (i.e., without motion correction). For example, the GUI may have a combination image comprising a deblurred image of the tumor within the BTZ, and a non-deblurred image outside the BTZ. The GUI may also provide notifications to the user while deblurring the PET images. For example, the GUI may have a motion indicator that notifies the user as to whether or not motion was identified in the expected frequency ranges in the PET imaging data. In one variation, if no motion is identified in the expected frequency range (e.g., no motion identified in the frequency ranges that correspond with physiological functions), a GUI having a first informational message may be output to a display to indicate that no motion has been detected. If motion is detected, a second informational message may be displayed to the user to indicate that motion has been detected.

The imaging data and motion data generated as part of deblurring a PET image may be used to generate a training data set that may be used to test and/or train one or more of the location estimation methods or algorithms. The training data set may also be used to generate an error metric or location uncertainty model, as described below. FIG. 8B is a flowchart depiction of one variation of a method for generating a training data set comprising PET imaging data and target region location for each motion phase of a target region. Method 820 may comprise decomposing 822 PET imaging data of a target region into a set of principal components using principal component analysis, identifying 824 a principal component from the set of principal components that has a dominant frequency similar to a predetermined frequency range, defining 826 a set of motion phases using the identified principal component, grouping 828 the PET imaging data into its corresponding motion phase within the set of motion phases, generating 830 phase-gated PET images by summing the PET imaging data at each motion phase, determining 832 a target region location for each motion phase by comparing the phase-gated PET images to a reference target location, and generating 834 a training data set comprising the PET imaging data for each motion phase and the determined target region location associated with that motion phase. In some variations, the training data set may comprise deblurred PET imaging data and target region location or shift data for each motion phase or bin. The descriptions for like steps in method 800 also apply to the corresponding steps in method 820. In some variations, the PET imaging data may comprise PET lines-of-response (LOR) data, and the training data set may comprise PET LOR data for each motion phase and the determined target region location associated with that motion phase. Alternatively, or additionally, the PET imaging data may comprise PET imaging data acquired during a series of acquisition time intervals, and the training data set may comprise PET imaging data acquired during specific acquisition time intervals within a motion phase and the determined target region location associated with that motion phase.

In some variations, the training data set may be used to train an adaptive correlation filter, as described above. Alternatively, or additionally, the training data set may be used to evaluate the location estimation accuracy of the maximum likelihood estimation methods and/or time-of-flight PET data centroid location methods.

In one variation, generating a reduced-motion image of the target region (referred to as a deblurred image) from an image having motion artifacts (e.g., motion blur) may comprise segmenting the target region and/or a contour around the target region (e.g., a biology-tracking zone BTZ contour around a gross tumor volume GTV contour) in a PET image into a set of PET activity isocontours that each define an isocontour shell, extracting the PET activity from the target region in each of the isocontour shells, setting the PET activity within the contour of the target region to the combined PET activity of the extracted target region PET activity from the set of isocontours, generating a background PET image by setting the PET activity of each isocontour shell to a background PET activity level, and generating a motion-reduced image by overlaying the contour of the target region (with the combined target region PET activity) on the background PET image. In one example, a target region may be defined by a target region contour and a BTZ contour may be defined to encompass the target region contour with an additional expansion to encompass the motion envelope of the target region. While the target region contour and the BTZ contour herein are depicted as 2-D contours, it should be understood that these are 3-D volumes and the contour is actually a 3-D surface. A PET image of the BTZ acquired over time may have motion artifacts that may cause the target region to appear blurred across regions of the BTZ. Without wishing to be bound by theory, because of the motion of the target region, the PET activity (or PET activity concentration) of the target region may be smeared in the BTZ. For any subregion in the BTZ, the total PET activity in that subregion may be a combination of background PET activity and tumor (target region) PET activity. In other words, the PET signal in the pixels/voxels of a BTZ subregion may be a combination of PET signals from the tumor and PET signals from non-tumor tissue (i.e., the background). The tumor PET activity in a subregion may be calculated by calculating or approximating the background PET activity, and then subtracting the background PET activity from the total PET activity in the subregion. Summing the tumor PET activity across all subregions may provide the total tumor PET activity within the BTZ. The total tumor PET activity from the PET image may be combined within the tumor contour and merged with the background PET activity levels for the remainder of the BTZ to generate a deblurred PET image of the tumor within the BTZ.

In one variation, the BTZ volume may be subdivided into a set of subregions that have similar PET activity or PET activity concentrations. These subregions may be referred to as isocontour shells of the PET activity concentration, where each isocontour shell contains regions having a similar range of PET activity or PET activity concentration level(s), each isocontour shell region bounded by its isocontour and the isocontour boundary of a neighboring isocontour shell region. The total PET activity or PET activity concentration for each isocontour shell may be a combination of background PET activity and tumor PET activity (tAᵢ). The aggregation of the tumor PET activity (tAᵢ) for all isocontour shells may provide an approximation of the total tumor activity (tA) in the BTZ. The total tumor activity tA over a number (i) of isocontour shells that comprise a BTZ may be calculated as

tA=ΣᵢtAᵢ (Equation 1)

where

$${tA}_{i} = ( 1 - \frac{bkgAC}{{shellAC}_{i}} ) {shellA}_{i} \qquad ( Equation 2 )$$

where i denotes the index of an isocontour shell, bkgAC is a background PET activity concentration, shellACᵢ is the PET activity concentration for isocontour shell i, and shellAᵢ is the PET activity for isocontour shell i. Both the PET activity concentration (shellACᵢ, with units of kBq/ml) and PET activity (shellAᵢ, with units of kBq) may be determined from the motion-blurred PET image (e.g., pixel/voxel intensity values) and/or the volume of the target region (e.g., as defined from a planning image, such as a CT image). In some variations, the background PET activity level or PET activity concentration level bkgAC may be calculated from a shell region outside the boundary of the BTZ, where there is little or no likelihood of tumor PET activity. The total tumor activity within the BTZ (tA), optionally modified by a scaling factor, may then be assigned as the tumor activity within the target region contour. The PET activity of the regions of the BTZ that do not comprise the target region/tumor (i.e., non-tumor tissue) may be replaced with a background level of PET activity. A reduced-motion or deblurred PET image may be generated by overlaying the target region contour having the determined tumor PET activity or PET activity concentration level on the background PET image where the remainder of the BTZ is at a background-level of PET activity and/or PET activity concentration.

While the various methods described herein may refer to PET activity concentration (AC) (having units of kBq/ml) or PET activity (having units of kBq), it should be understood that the methods may use either or both quantities to describe the amount and/or intensity of PET signals measured and depicted in a PET image. The intensity values at each pixel or voxel of a PET image may indicate and/or be used to calculate the PET activity concentration and/or PET activity at that pixel or voxel. While the unit analysis for a calculation may be adjusted depending on which quantity (PET activity concentration or PET activity) is being used, the methods provided herein may be described using one or both of PET activity and/or PET activity concentration and/or PET signal intensity. In some variations, “PET activity” and “PET activity concentration” and “PET signal” and “PET signal intensity” may be used interchangeably, and are all understood to be quantities that may be calculated using the pixel/voxel data of a PET image.

FIG. 8C is a flowchart depiction of one variation of a method for generating a reduced-motion (e.g., “deblurred”) PET image, which may be used for radiotherapy treatment planning. Method 840 may comprise calculating 842 a background level of PET activity in a first PET image of a tumor within a BTZ contour, where the tumor has a tumor contour and an initial PET activity within the tumor contour, defining 844 a set of PET activity isocontours in the first PET image, where the isocontours delineate a corresponding set of isocontour shells within the BTZ contour, calculating 846 for each isocontour shell, the PET activity originating from the tumor, calculating 848 a total tumor PET activity (tA) within the BTZ contour by summing the tumor PET activity in all isocontour shells, generating 850 a background PET image by setting the PET activity for the isocontour shells to a background level of PET activity, adjusting 852 the initial PET activity within the tumor contour to have the calculated total tumor PET activity, and generating 854 a second PET image of the tumor (e.g., a reduced-motion or deblurred PET image) by combining the tumor contour with the adjusted PET activity and the background PET image. Optionally, method 840 may further comprise generating 856 a radiotherapy treatment plan using the second PET image.

The tumor contour may be defined using a CT image, for example, by drawing an outline around the tumor in a CT image. In some variations, the acquisition time for the CT image may be relatively short such that there is little or no motion artifacts in the CT image. Alternatively, or additionally, the CT image may be a post-processed image such that motion and/or other artifacts are removed. Conceptually, the CT image is a “motion frozen” or “motion free” image. The tumor contour may be, for example, a GTV (gross tumor volume) contour. FIG. 8D is a conceptual depiction of the various contours and shell regions used in the method 840 of FIG. 8C. These contours and shell regions may be defined in the first PET image, and the PET activity (e.g., PET activity concentration) of these shell regions may be calculated from the intensity values at each pixel/voxel of the first PET image. As depicted in FIG. 8D, the BTZ contour 860 may be sized and shaped to enclose the tumor or GTV contour 862 along with an expansion to include the potential motion trajectory of the tumor 862. A nominal value of PET activity within the tumor contour may be derived by overlaying and aligning the initial (e.g., blurred) PET image with the CT image, and calculating the PET activity in the region enclosed by the tumor contour. Calculating 842 a background level of PET activity (e.g., PET activity concentration, PET signal, etc.) may comprise defining a shell region around the BTZ contour, such as a shell region just outside the BTZ contour, and calculating the mean PET activity (e.g., PET activity concentration, PET signal) in this shell region. The shell region does not include the region enclosed within the BTZ contour. For example, FIG. 8D depicts an outer shell region 861 that is just outside the BTZ contour 860. Since the BTZ contour 860 encloses the most likely locations of the tumor contour 862, it is unlikely that any PET signals originating from the tumor will be present in the outer shell region 861. As such, a background PET activity concentration (e.g., background PET signal) may be calculated from the mean (i.e., average) PET activity concentration (e.g., mean or average PET signal) of the outer shell 861 using the pixel/voxel values in the first PET image. Alternatively, the background PET activity or PET signal level may be calculated by subtracting the PET activity or signal originating from the tumor from the total PET activity within the BTZ contour and/or one or more isocontour shells.

PET activity isocontours (or PET activity concentration isocontours) may be defined 844 using the pixel/voxel values in the first PET image, where the pixel/voxel values may represent PET activity, PET activity concentration, and/or PET signal intensity values. The isocontours may be the boundaries that define a plurality of isocontour shells. FIG. 8D depicts an example where there are 7 PET activity isocontours that define 8 isocontour shells 863-870. In this example, the innermost isocontour shell 870 may not contain any isocontours or isocontour shells. The PET activity isocontours may be within the BTZ contour, and the PET activity in each isocontour shell may be relatively similar and/or may be within a predefined range. For example, the PET activity within each isocontour shell may be within a specified range around the mean PET activity level in the shell. The mean PET activity may be different for each different isocontour shell. While the example depicted in FIG. 8D has 8 concentric isocontour shells that have shapes that are derived from the shape of the BTZ contour 860, it should be understood that in other examples, the isocontour shells may not be concentric and/or nested. The shapes of the isocontour shells may or may not be similar to the shape of the BTZ contour and/or the tumor/GTV contour. There may be any number of isocontours and corresponding isocontour shells, depending on the relative size difference between the tumor contour and the BTZ contour. In some variations, the greater the differences between the volume of the tumor and the volume of the BTZ, the more isocontours and isocontour shells may be defined. The number of isocontours and isocontour shells may be determined, at least in part, on the homogeneity of the PET signal (e.g., PET activity, PET activity concentration, etc.) within the BTZ. If there is less PET signal variation in the BTZ region, there may be fewer isocontours and isocontour shells than if there is greater PET signal variation.

Calculating 846 the PET activity (e.g., PET activity concentration) originating from the tumor for a particular isocontour shell may comprise subtracting a background level of PET activity from the PET activity in the isocontour shell. In some variations, the background level of PET activity may be estimated from the outer shell 861 surrounding the BTZ. Calculating 846 the tumor PET activity for an isocontour shell of a PET image may use the pixel/voxel data to calculate PET activity and PET activity concentration within the isocontour shell and/or a background of the PET image, and using Equation 1 to calculate the PET tumor activity. Calculating 848 the total tumor PET activity (e.g., PET signal, PET activity concentration) within the BTZ may comprise adding together the PET activity calculated for each isocontour shell. In some variations, the second PET image comprises the PET activity/signal/activity concentration in the region of the patient that is within the BTZ contour, and the PET activity/signal/activity concentration in the in region enclosed by the tumor contour, where the tumor contour is at a selected location within the BTZ contour and is not blurred or smeared across the BTZ region. To construct such a PET image, in some variations, the PET activity/signal/activity concentration of the non-tumor region of the BTZ may be set to a background PET activity/signal/activity concentration level, while the PET activity/signal/activity concentration of the tumor is set to the calculated total tumor PET activity/signal/activity concentration (e.g., as calculated in 848). The background PET activity/signal/activity concentration of one or more isocontour shells may be set 850 to the same background PET activity/signal/activity concentration as the outer shell region 861. Alternatively, or additionally, the PET activity/signal/activity concentration of one or more isocontour shells may be set 850 to the PET activity/signal/activity concentration of that isocontour shell minus the PET activity/signal/activity concentration of the tumor region. In some variations, generating 850 a background PET image may be optional, or generating 850 a background PET image may comprise an image where the pixel/voxel value outside of the tumor contour has a nominal value, e.g., 0 or 100, that provides a significant contrast with the adjusted PET signal values within the tumor contour from step 852.

In some variations, adjusting the initial PET activity within the tumor contour may comprise normalizing the PET activity within the tumor contour to the total tumor PET activity, such that a total PET activity within the tumor contour matches the calculated total tumor PET activity. For example, this may comprise calculating a scaling factor for each pixel/voxel within the PET image of the tumor contour, applying the scaling factor to the total tumor PET activity to attain a normalization offset, and adding or subtracting the normalization offset to the initial PET activity at that pixel/voxel. The scaling factor for a pixel in within the tumor contour may be a ratio of the initial PET activity/activity concentration/signal to the initial total tumor PET activity. The normalization offset for the pixel may be calculated by multiplying the scaling factor by the total tumor PET activity (calculated in step 848, for example). The normalization offset may be applied (added or subtracted or adjusted by or modulating) to the initial PET activity/activity concentration/signal for the pixel. Alternatively, or additionally, adjusting 852 the initial level of PET activity within the tumor contour may comprise calculating a scaling factor based on a ratio of the calculated total tumor PET activity to the initial level of PET activity, calculating a PET activity offset by multiplying pixel values within the tumor contour by the scaling factor, and modulating the pixel values within the tumor contour by the PET activity offset. Generating the second PET image may comprise overlaying the tumor contour with the adjusted PET activity onto the background PET image of the BTZ. The tumor contour with the adjusted PET activity may be placed anywhere within the BTZ, for example, the regions of the BTZ where the tumor is likely to have the longest dwell time, and/or the center region of the BTZ.

FIG. 8E depicts a flowchart representation of a variation of the method 840. Method 880 may comprise defining 882 a tumor contour in a CT image and an initial PET activity within the tumor contour in a first PET image of the tumor in a BTZ contour, calculating 884 a background PET activity concentration (bkgAC) by taking the mean of the PET activity concentration in a shell outside the BTZ contour, defining 886 a set of (i) isocontours and isocontour shells within the BTZ contour based on regions of similar PET activity concentrations, calculating 888, for each isocontour shell (i), the PET activity (tAᵢ) originating from the tumor using Equation 1, calculating 890 a total tumor PET activity (tA) within the BTZ contour by summing the tumor PET activity in all isocontour shells using Equation 2, generating 892 a background PET image by setting the PET activity for the isocontour shells to a background level of PET activity, adjusting 894 the PET activity concentration within the tumor contour according to the calculated total tumor PET activity, and generating 896 a second PET image of the tumor by combining the tumor contour with the adjusted PET activity and the background PET image. Optionally, method 880 may comprise generating a radiotherapy treatment plan using the second PET image. One or more steps in method 880 may comprise one or more of the methods described above for the corresponding steps in method 840.

Methods 800, 820, 840, 880 may be used to process images/imaging data for treatment planning and/or testing or training before a treatment session, but may also be used to process images/imaging data acquired at the beginning of a treatment session (e.g., radiation delivery session). For example, because biology-guided radiotherapy uses PET emission data (i.e., PET imaging data) to guide delivery of the radiation fluence to the patient, at the beginning of the treatment session, there may be one or more evaluations of the PET imaging data to confirm that it is of sufficient quality to safely guide delivery of radiation. A PET prescan may be conducted at the beginning of the treatment session to acquire PET imaging data using the on-board radiotherapy system PET imaging system. If the PET imaging data at the treatment session is substantially different from the PET imaging data used for treatment planning, the radiotherapy system controller may generate notifications that inform the clinician of the differences, and may recommend pausing radiation delivery. For example, if the PET imaging signal from the target region is much less bright, has lower contrast, lower activity concentration, than the PET imaging signal from the target region during treatment planning, the radiotherapy system controller may provide that indication to the clinician and offer an option to stop or not proceed with treatment. Methods 800, 820 may be applied to PET prescan imaging data to generate a deblurred PET image (method 800) and/or generate a testing data set (method 820), and one or more of the location estimation methods intended to be used during the radiotherapy treatment session to guide the delivery of radiation may be applied to the testing data set. The resulting location estimates from the different location estimation methods may be compared to the known initial location of the target region (e.g., derived from the deblurred PET image), and the differences between the known initial location and the location estimates may be used by the controller to calculate an error metric for each location estimation method. In some variations, the error metric value(s) for a location estimation method calculated at the beginning of a treatment session may be compared to the error metric value(s) for the same location estimation method calculated before the treatment session, e.g., as part of treatment planning, testing and/or quality assurance procedures. In one example, the error metric may comprise an error bar, and the error bar derived during a treatment session may be compared with the error bar derived during planning/testing/quality assurance procedures, and if the error bar differs beyond an acceptable tolerance, the radiotherapy system controller may generate a notification to the clinician that indicates this difference, and providing an option to pause or not start radiotherapy treatment. Alternatively, or additionally, PET deblurring ratio, the spread and/or the mean value of the uncertainty model derived from the PET prescan imaging data (e.g., using the methods described herein) may be evaluated, as described below. The method described above uses the phases detected by PET deblur as a replacement for the actual ground-truth motion trajectory to calculate one or more location estimation quality metrics (e.g., location error metrics, uncertainty clouds, confidence metrics, etc.).

In some variations, the quality of a PET deblur method may be quantified by a PET deblurring ratio. The PET deblurring ratio may be defined as the ratio of the activity concentration (AC) of the target region in the deblurred PET image to the AC of the target region in the motion averaged (e.g., not motion-corrected) PET image. Some methods may optionally comprise evaluating the quality of a PET deblur method before moving forward with treatment planning or delivery. For example, if the quality of the PET deblur method is not satisfactory, the treatment planning workflow does not proceed with the optimization process. If the motion artifacts in a PET image cannot be adequately corrected or removed, the treatment planning and/or delivery system may not be able to accurately determine the location of the target region(s), and may not deliver the desired/prescribed dose to the target region(s) and/or deliver dose to non-target tissue. The AC value of a target region in a PET image may be calculated from the pixels/voxels enclosed by the contours (i.e., boundaries) of the target region. A PET deblurring ratio of about 0.8 or 0.9 or more may indicate that the deblurring process has removed and/or corrected enough of the motion artifacts in the PET images such that the location estimation methods are able to provide reasonably accurate location estimates. A PET deblurring ratio that is less than about 0.8 or 0.7 may indicate that the deblurring process has failed.

### Location Uncertainty Model and Tracking Error Metric

The precision and/or accuracy of the location estimator methods described herein may be evaluated and/or characterized as part of a radiotherapy treatment planning and/or treatment session workflow. In some variations, the precision and/or accuracy of a location estimator method or algorithm may be represented in one or more location estimation quality metrics, such as a confidence metric, uncertainty metric or model, and/or error metric (e.g., error bar). An uncertainty model may characterize the precision of a location estimator method, and may comprise, for example, an uncertainty cloud. An error metric and/or uncertainty model for a location estimator method may be calculated by applying the location estimator to a set of imaging data for which the location of the target region is known (e.g., using the deblurring methods described herein, or any other methods where imaging data is acquired in synchrony with a motion or position sensor). The location estimation quality metric(s) (e.g., error metric, uncertainty model) for a location estimator method may be calculated before a treatment session, for example, as part of treatment planning and/or testing and/or quality assurance procedures. The location estimation quality metric(s) for the same location estimator method may be calculated at the beginning of a treatment session, and optionally, at additional timepoints during the treatment session. For example, the location estimation quality metric(s) for a location estimator method may be calculated using PET imaging data acquired during a planning PET imaging session, and/or using PET imaging data acquired during a PET prescan in a treatment session. This can be done during a functional modeling session or PET prescan. In some variations, the PET imaging data acquired during a PET prescan at the beginning of a treatment session may be used to update one or more of the location estimation methods described herein.

As described above and herein, the one or more location estimation algorithms may be trained on imaging data as part of treatment planning. Adaptive correlation filters (or templates) may be generated for the ACF location estimator, probability density functions may be generated for the MLE location estimator, settings of the Kalman filter, and target region anchor(s) and other treatment parameters may be defined and/or initialized using imaging data acquired or otherwise provided during treatment planning. The location estimators may be applied to additional imaging data (e.g., testing data, training data) to evaluate their performance, for example, to characterize and evaluate their precision in identifying a target region location. These characterization and/or evaluations may be used to derive a confidence metric that a user may use to decide on whether to rely upon the output of the location estimation methods to deliver radiation. Testing, evaluating and/or characterizing the precision of one or more location estimation methods may comprise applying the location estimators to imaging data where the location of the target region within the imaging data is known to generate a location estimate, and comparing the generated location estimate with the known location. This testing data (which may be referred to as training data) may include “ground truth” information on the target region motion, shift and/or location data. Such “ground truth” information may be derived by simulation by a computer model, empirical measurements (e.g., where imaging data and tumor position data acquired simultaneously), calculations from empirical measurements (e.g., acquired imaging data is processed and analyzed to determine tumor position data), and/or a combination of simulation and empirical data. Generating a location uncertainty model and/or tracking error metric may comprise calculating the difference(s) between the location(s) of the target region as estimated by the estimation algorithms and the “ground truth” location(s) in the training data. In some variations, the location uncertainty model may comprise an uncertainty cloud that includes the estimated locations of the target region from the location estimator algorithms superimposed over the “ground truth” location of the target region. The size (e.g., spread) of the uncertainty cloud and the average value of the differences between the estimated location(s) and the “ground truth” locations (which may optionally be weighted based on the likelihood of a tumor location being at a particular point in the uncertainty cloud) may be characteristics that are stored in the memory of a controller/processor. During treatment planning, the uncertainty cloud may be used to generate the planned firing filters during treatment plan optimization (e.g., firing filters for each firing position of the therapeutic radiation source). During a radiation delivery session (e.g., a treatment session), these characteristics may be retrieved and compared to a location uncertainty cloud that is calculated from applying the location estimators to the imaging data acquired at the beginning of the radiation delivery session (e.g., imaging data from a PET prescan, before radiation is emitted). The characteristics of the uncertainty cloud calculated at the time of treatment may be compared to the characteristics of the uncertainty cloud calculated during treatment planning. For example, the size (e.g., spread) and the average value of the uncertainty cloud calculated during the radiation delivery session may be compared to the same characteristics that were calculated treatment planning. If the differences between these characteristics exceed a threshold, and/or if the uncertainty cloud calculated during the radiation delivery session has a size/spread that is too large, a user may decide not to proceed with the radiation delivery.

FIG. 9A depicts a flowchart representation of one variation of a method for calculating a location estimation quality metric, e.g., a location uncertainty model, tracking error metric, for a location estimator method or algorithm (e.g., a maximum likelihood estimation methods, time-of-flight PET data centroid locator methods, adaptive correlation filter methods, template matching methods, Kalman filter method that combines the location estimates of multiple locator methods, etc.). Although the examples provided herein pertain to PET imaging data, it should be understood that these methods may also be applied to other imaging modalities, including CT imaging data, MR imaging data, optical imaging data, ultrasound data, and the like. While the method calculates a tracking error metric as an example, it should be understood that the method may calculate any one or more of the location estimation quality metrics described herein, including, for example, a location uncertainty model. Method 900 may comprise acquiring 902 PET images/imaging data of a target region over a plurality of motion phases, identifying 904 the location of the target region at each of the plurality of motion phases, applying 906 a location estimator algorithm to the PET images/imaging data to calculate an estimated location of the target region at each motion phase, and calculating 908 a tracking error metric based on a comparison of the estimated location with the identified location of the target region at each motion phase. In some variations, training data sets generated using the image deblur methods described herein (e.g., methods 800, 820) may be used to test the location estimator algorithm. Steps 902 and 904 may be optional. The PET images/imaging data may be acquired using a standalone PET imaging system or a PET imaging system that is onboard a radiotherapy system. The PET image/imaging data acquisition may occur as part of treatment planning (where no therapeutic radiation is delivered to the patient), and/or as part of a PET prescan that occurs during a treatment session. The PET images/imaging data may be motion-corrected and/or deblurred (e.g., using any of the image deblur methods described above). Alternatively, or additionally, the PET imaging/imaging data may not be motion-corrected and/or deblurred. The number and/or extent of motion phases may be defined by a clinician or using a principal component analysis as described above. The motion of the target region may be periodic or aperiodic. Identifying 904 the location of the target region may comprise using any of the image deblur methods described above (e.g., methods 800, 820), which may include calculating a target region shift for each of the motion phases. The tracking error metric may be an error bar representing the variation of a location estimate, and/or the range of possible of target region locations given the PET images/imaging data. While method 900 comprises calculating 908 a tracking error metric, it should be understood that this step may alternatively, or additionally, comprise calculating 908 a location uncertainty model (e.g., location uncertainty cloud), as described further below. In some variations, the tracking error metric may be used to adjust the image of the tumor location, and this adjusted image may then be used for treatment planning. Optionally, after a treatment session where radiation was delivered to a patient using the location information about the target region(s), some methods may comprise checking the tracking error metric calculated prior to delivery with the tracking error metric calculated after delivery.

In some variations, a tracking error metric may comprise an error bar derived from the difference between a target region location estimated by a location estimator algorithm, and the actual “ground truth” target region location. Similarly, a location uncertainty model may comprise an uncertainty cloud derived from the difference between a target region location estimated by a location estimator algorithm, and the actual “ground truth” target region location. The “ground truth” target region location may be calculated using motion and/or position data acquired concurrently with image acquisition, and/or may be the target region location in a motion phase that is calculated as part of an image deblurring method. For example, one variation may comprise deblurring or correcting motion artifacts from PET imaging data acquired during treatment planning, defining a plurality of motion phases of the target region, grouping or assigning an increment of PET imaging data to a motion phase, aligning the PET imaging data of all motion phases to a single phase in a straight line, and summing the PET imaging data to attain a final image of the target region at the location of the single phase. The PET imaging data (which may be PET imaging data acquired over a specified acquisition time window, such as limited time sampled PET LTS imaging data) may be used to test a location estimator algorithm, and the resultant estimated location may be compared to the final image of the target region at the location of the single phase. The difference between the estimated location and the final image of the target region may be used to derive the error bar. In some variations, a location estimation quality metric such as a location uncertainty model may be generated by applying the one or more location estimator algorithms to the motion deblurred and/or motion-corrected PET LTS data acquired during a planning imaging session to estimate the relative shifts (or residual errors) of the deblurred and/or motion-corrected LTS images (which are partial PET images) with respect to the full deblurred and/or motion-corrected PET image of the target region.

Optionally, when used in the context of a PET prescan at the beginning of a biology-guided radiotherapy treatment session, method 900 may comprise comparing 910 the tracking error metric with an expected tracking error metric to determine whether to proceed with radiation delivery. In some variations, the method may comprise calculating a location uncertainty model from applying the location estimator methods to the PET prescan to obtain a PET prescan location uncertainty cloud, and comparing characteristics of the PET prescan uncertainty cloud with the characteristics of the planning uncertainty cloud calculated during treatment planning. For example, the size/spread of the PET prescan uncertainty cloud and/or the average value of the planning uncertainty cloud may be compared before a radiation delivery session. As explained above, if calculated tracking error metric differs substantially from the expected tracking error metric, the method may comprise generating a GUI that includes a graphical element that indicates a change in the patient and/or PET imaging data that may need additional safety review to confirm whether radiation delivery would provide a safe and conformal dose to the target region. In some variations, if the characteristics of the PET prescan uncertainty cloud and the planning uncertainty cloud differ substantially and/or have a difference that exceeds an acceptable range (e.g., the differences in the spread/size of the cloud and/or average value of the cloud are not within an acceptable range), the method may comprise generating a GUI that includes a graphical element that indicates a change in the patient and/or PET imaging data that may need an additional safety review to confirm whether to proceed with radiation delivery. Optionally, some methods may comprise updating and/or training the one or more location estimator algorithms using the PET prescan imaging data. The characteristics of the PET prescan uncertainty cloud may be re-calculated for the updated location estimator algorithms and re-evaluated to determine whether the differences from the planning uncertainty cloud are within the acceptable range. The method may provide an additional GUI that indicates whether radiation delivery may proceed with the updated/re-trained location estimator algorithms.

One variation of a method for generating an uncertainty model (such as an uncertainty cloud) may comprise deblurring PET LTS images acquired in a planning imaging session, aligning the PET LTS images across the motion trajectory of the target region to a single motion phase or bin, training the location estimator algorithms using the aligned, deblurred PET LTS images, applying the location estimator algorithms to the aligned, deblurred PET LTS images to calculate a set of target region shifts across the motion trajectory of the target region, and generating an uncertainty model (e.g., an uncertainty cloud) that includes the set of calculated shifts. Without wishing to be bound by theory, in an ideal scenario where the PET deblur perfectly removes all the motion artifacts in the PET LTS images, location estimation methods trained on such images would work perfectly and detect no shifts (i.e., shift values of zero or nearly zero) when applied to aligned, deblurred PET LTS images. However, in reality, the PET deblur process does not remove all motion artifacts in the PET LTS images, and so any non-zero detected shifts from applying the location estimation method may be considered an “error”. The pattern of non-zero shifts may be errors that are expected from the location estimation method(s). The pattern of non-zero shifts may comprise points in the uncertainty cloud. FIG. 9B depicts a variation of a method for generating a location (e.g., tracking) uncertainty model for radiotherapy treatment planning and radiation delivery evaluation. Method 920 may comprise acquiring 922 PET images/imaging data of a target region over a motion trajectory of the target region (e.g., over a plurality of motion phases), deblurring 924 the PET images/imaging data of the target region, training 926 the location estimator methods using the deblurred target region imaging data, applying 928 the location estimator methods to the deblurred target region imaging data to determine a set of estimated target shifts, and generating 930 a tracking uncertainty model from the set of estimated target shifts. The acquired PET images/imaging data may be PET LTS images, or any series of PET images or imaging data acquired over a pre-determined interval of time, which may contain any of the motion artifacts described above. The imaging data may be deblurred 924 using any of the methods described herein, for example, as described in FIGS. 8A-8E, and may comprise aligning images of the target region from different motion phases or bins to a single phase or bin. The location estimator methods may be trained 926 using deblurred imaging data that have been aligned to a motion phase or bin. The tracking uncertainty model may be an uncertainty cloud that comprises points representing the set of estimated target shifts. The uncertainty model may have a size/spread (e.g., the largest separation distance between points in the cloud) and an average value (e.g., the average of the shift values in the cloud).

One variation of a method for generating an uncertainty model (such as an uncertainty cloud) may comprise deblurring PET LTS images acquired in a planning imaging session, and aligning the PET LTS images across the motion trajectory of the target region to a single motion phase or bin, training the location estimator algorithms using the aligned, deblurred PET LTS images, and applying the location estimator algorithms to the aligned, deblurred PET LTS images to calculate a first set of target region shifts across the motion trajectory of the target region, as depicted in FIG. 9B and described above. However, alternatively and/or additionally to applying the location estimator algorithms to the aligned, deblurred PET LTS images, one variation may comprise applying the location estimator algorithms to the uncorrected PET LTS images (e.g., the blurred or original PET LTS images containing motion artifacts, PET LTS images that are not aligned across multiple motion phases or bins) to calculate a second set of target region shifts across the motion trajectory of the target region. The method may comprise calculating the shift differences between the first set of target region shifts and the second set of target region shifts, and generating an uncertainty model (e.g., an uncertainty cloud) that includes the shift differences. For example, calculating the shift differences may comprise subtracting the target region shifts in the second set from the target region shifts in the first set. Without wishing to be bound by theory, generating an uncertainty model by applying the location estimator algorithms on non-aligned, non-deblurred (i.e., uncorrected) PET LTS images may help provide an uncertainty model that is less susceptible to biases and/or assumptions that may underpin one or more location estimator algorithms. This may generate an uncertainty model that is a better approximation of the uncertainty, precision, and/or confidence of the one or more location estimation methods because the uncertainty or errors are measured against images and/or imaging data that better resembles the images and/or imaging data that may be acquired during a radiation delivery session. FIG. 9C depicts a variation of a method for generating a location (e.g., tracking) uncertainty model for radiotherapy treatment planning and radiation delivery evaluation. Method 940 may comprise acquiring 942 PET images/imaging data of a target region over a motion trajectory of the target region (e.g., over a plurality of motion phases), deblurring 944 the PET images/imaging data of the target region, training 946 the location estimator methods using the deblurred target region imaging data, applying 948 the location estimator methods to the deblurred target region imaging data to determine a first set of estimated target shifts, applying 950 the location estimator methods to the uncorrected (e.g., not deblurred) PET images/imaging data of the target region to determine a second set of estimated target shifts, and generating 952 a tracking uncertainty model from the first and second set of estimated target shifts by calculating shift differences between the first and second set of estimated target shifts. In some variations, calculating shift differences may comprise subtracting the second set of estimated target shifts from the first set of estimated target shifts. The acquired PET images/imaging data may be PET LTS images, or any series of PET images or imaging data acquired over a pre-determined interval of time, which may contain any of the motion artifacts described above. The imaging data may be deblurred 944 using any of the methods described herein, for example, as described in FIGS. 8A-8E, and may comprise aligning images of the target region from different motion phases or bins to a single phase or bin. The location estimator methods may be trained 946 using deblurred imaging data that have been aligned to a motion phase or bin. The tracking uncertainty model may be an uncertainty cloud that comprises points that include the calculated shift differences 952. Each point in the uncertainty cloud may have a value that indicates the difference between the target region shift determined from applying the location estimators to deblurred imaging data and the target region shift determined from applying the location estimators to uncorrected imaging data. The uncertainty model may have a size/spread (e.g., the largest separation distance between points in the cloud) and an average value (e.g., the average of the shift values in the cloud).

One example of a method for generating a location uncertainty model (which may also be referred to as a location “confidence model”) for one or more trained/generated location estimator algorithms may comprise applying the one or more location estimator algorithms to a series of TOF-FBP reconstructed (e.g., SSRB-FBP) PET LTS images (e.g., acquired during a planning imaging session) to estimate the target region position at each time point tk, generating a “measured” trajectory of the target region, where the “measured” trajectory comprises a series of time points tₖ and corresponding estimated target region position or shift

$${p}_{k}^{measured} ,$$

calculating the tracking error as a vector difference between the “measured” trajectory

$$( {t}_{k} , {p}_{k}^{measured} )$$

and a “ground truth” trajectory

$$( {t}_{k} , {p}_{k}^{gnd truth} ) ,$$

and building the uncertainty model as a volume (e.g., uncertainty cloud) by placing a 3D anchor function at the planned target position and at different shifted locations as determined by the vector differences. The “ground truth” trajectory may be obtained empirically, for example, by acquiring target region location data concurrently during image acquisition, or may be calculated using any of the deblurring methods described herein. The vector difference or error ek may be calculated by subtracting (i.e., taking the difference between) the “measured” position and the “ground truth” position, e.g.,

$${e}_{k} = {p}_{k}^{measured} - {p}_{k}^{gnd truth} .$$

The uncertainty cloud may indicate the most likely locations of the target region assuming the target region was at the planned target position. In some variations, the 3D anchor function may be a 3D Gaussian distribution.

Alternatively, or additionally, to applying the trained location estimator methods to (optionally aligned) deblurred target region imaging data, and/or uncorrected target region imaging data, some methods for generating an uncertainty model may comprise applying the trained location estimator methods to simulated imaging data derived from the acquired images/imaging data of the target region. The simulated imaging data may be generated by deblurring the target region imaging data and aligning the deblurred imaging data from different motion bins to a reference target location (e.g., a target location in a single bin), and applying a shift value to the imaging data in each of the motion bins. The imaging data and/or images of the target region may be acquired over a motion trajectory of the target region. The shift value may be randomly selected, and in some variations, may be limited to physiologically likely shifts and/or shifts that are still within a user-defined contour (e.g., such that the PTV is still within the BTZ, and/or applying a corresponding circshift to the imaging data). The shift values for the imaging data across all motion bins may be derived from a motion trajectory activity of other imaging data, other patients, and/or other physiological functions. This simulated imaging data may contain images/imaging data acquired by an imaging system, but introduce additional or alternative shifts or motion patterns that may be different from the motion of the target region during the image acquisition. To generate an uncertainty model, one variation may comprise training the location estimator algorithms on the simulated imaging data, applying the trained location estimator algorithms to the simulated imaging data to determine a set of estimated simulated target shifts, and generating the uncertainty model from the set of estimated simulated target shifts. The tracking or location uncertainty model may be an uncertainty cloud that comprises points representing the set of estimated target shifts. The uncertainty model may have a size/spread (e.g., the largest separation distance between points in the cloud) and an average value (e.g., the average of the shift values in the cloud). Alternatively, or additionally, the set of estimated simulated target shifts may be subtracted from the set of target shifts determined by applying the location estimator method(s) to deblurred target region imaging data.

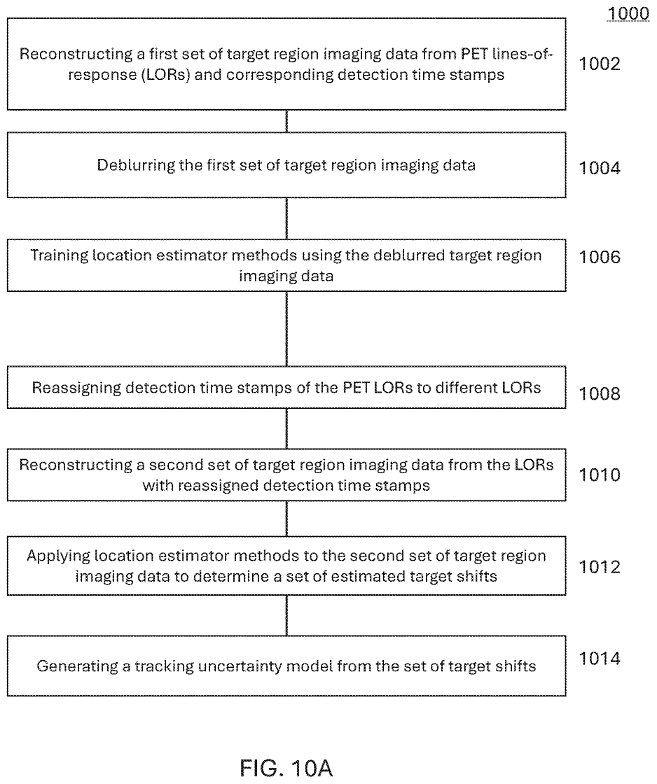

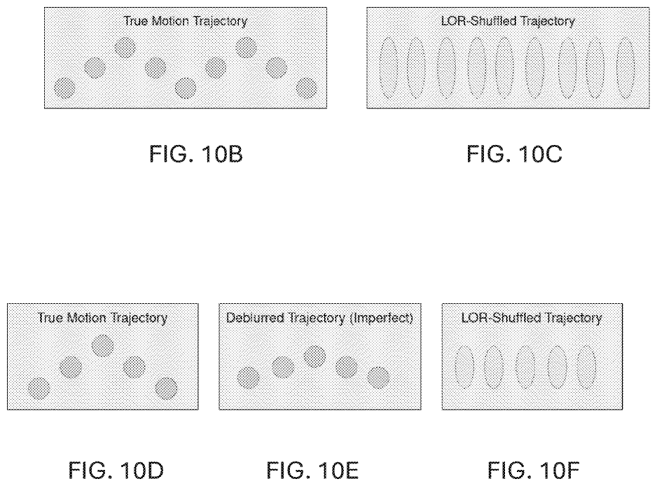

The target region imaging data and/or images may comprise a series of LORs with a corresponding time stamp for each LOR indicating the time at which the LOR was detected. PET images (e.g., LTS images) may be reconstructed from the series of LORs, using the time stamp data to reconstruct a motion trajectory. FIG. 10B is a conceptual diagram of LTS images of a target region reconstructed from LORs and their corresponding (original) time stamps, where the original motion pattern is retained in the image. Each LTS image (represented by a circular shape) in FIG. 10B represents a dynamic, but blurred image of the target region. However, in some scenarios, it may be possible that motion artifacts are not completely removed during the deblurring process, which may result in some inaccuracies and/or artifacts in the corrected/deblurred image. When location estimation methods are trained on these images, in some situations, the artifacts may be further exacerbated, especially if the location estimation methods are susceptible to the errors arising from motion artifacts. For example, if the deblurring methods and location estimator methods have correlated motion errors, this may result in inaccurate target region location estimates and/or an inaccurate uncertainty model. One approach to addressing these possible motion-related errors or inaccuracies may be generated simulated imaging data that removes the original motion pattern from the PET imaging data and apply the trained location estimation methods to the PET imaging data without the motion pattern to generate the location/tracking uncertainty model. In one variation, removing the original motion pattern may comprise re-assigning or shuffling the time stamps of the LORs, where each LOR is assigned a time stamp from another LOR and/or a time stamp that is different from its original time stamp. The resultant simulated imaging data may comprise a series of LORs that each have a time stamp that is different from their original time stamp. FIG. 10C is a conceptual diagram of LTS images reconstructed from LORs and their shuffled time stamps, where the original motion pattern is no longer retained in the image. Each LTS image (represented by an oblong shape) in FIG. 10C represents a static, blurred image of the target region. In this example, the image of the target region may be more blurred than if the LORs had their original time stamps, however, the motion pattern is removed so that any artifacts or errors correlated with motion are reduced or eliminated. One variation of a method of generating an uncertainty model using simulated imaging data (e.g., LORs with shuffled time stamps) may comprise training the location estimator algorithms on aligned, deblurred PET images that have been reconstructed from a series of PET LORs where each LOR has a timestamp associated with the detection of the photons that define that LOR, creating simulated imaging data having a series of PET LORs where the original time stamp of each LOR is replaced (e.g., shuffled) with a different time stamp, reconstructing LTS images from the simulated imaging data, applying the trained location estimator algorithms on the reconstructed LTS images to determine a set of estimated target region shifts, and generating a location uncertainty cloud from the set of estimated target region shifts. In some variations, the estimated target region shifts in the set comprise the points in the uncertainty cloud. Generating an uncertainty model based on simulated imaging data that has no motion pattern may help reduce or eliminate inaccuracies that may arise from motion-correlated errors and/or scenarios where PET images fail to be deblurred and/or motion-corrected. Without wishing to be bound by theory, the simulated imaging data comprising PET LORs with shuffled time stamps may be a “ground truth” data set whereby there is little or no motion, in contrast with deblurred PET images which still include motion data which may or may not be accurate depending on whether the deblurring process accurately reduced or removed motion. Shuffling the time stamps of the PET LORs may entirely remove the motion pattern, resulting in a PET LTS collection with no expected phase difference from one LTS to the next.

FIG. 10A depicts one variation of a method for generating a location/tracking uncertainty model using simulated imaging data reconstructed from PET LORs with shuffled time stamps. Method 1000 may comprise reconstructing 1002 a first set of target region imaging data from PET lines-of-responses (LOR) and corresponding detection time stamps, deblurring 1004 the first set of target region imaging data, training 1006 one or more location estimator methods using the deblurred target region imaging data, reassigning 1008 detection time stamps of the PET LORs to different LORs, reconstructing 1010 a second set of target region imaging data from the LORs with reassigned detection time stamps, applying 1012 the trained one or more location estimator methods to the second set of target region imaging data to determine a set of estimated target shifts, and generating 1014 a tracking uncertainty model (e.g., uncertainty cloud) from the set of target shifts. The deblurring 1004 and training 1006 steps may be performed using any of the methods described herein. For example, deblurring 1004 the first set of target region imaging data may comprise aligning images of the target region from different motion phases to a single motion phase and/or reference target location, and determining a target region shift for each motion phase of the target region. The first and second set of target region imaging data may each be a series of PET LTS images. Reassigning 1008 the detection time stamps of the PET LORs may comprise randomly assigning the detection time stamps of different PET LORs in the same imaging data set, and/or assigning each LOR to an arbitrary different time stamp unrelated to the time stamps of the LORs in the same imaging data set.

In some variations, reassigning 1008 the detection time stamps of the PET LORs may comprise reassigning the time stamps of the uncorrected and/or unprocessed (e.g., “raw”) LORs, but in other variations, the reassignment of time stamps may be done for PET LORs that have been motion-corrected (i.e., deblurred). One variation for deblurring (i.e., motion-correcting) PET LORs may comprise deblurring the PET LTS images that have been reconstructed from non-deblurred (e.g., “raw”, uncorrected) PET LORs to determine the motion phase or bin for each PET LTS image, determining a target region shift for each motion phase as compared to a reference target location, and shifting each LOR in each motion phase by the inverse of determined target region shift for that motion phase. Shifting each LOR to compensate for the target region shift may remove the displacement of the LOR resulting from the target region motion, thereby removing the motion component and “deblurring” or motion-correcting the LOR (i.e., a “static” LOR with the motion displacement compensated for, instead of a “dynamic” LOR which includes a displacement from a reference target location due to the target region motion). In some variations, the deblurring and/or motion-correction of PET LORs may be performed for one axis or component of the motion trajectory. For example, some variations may motion-correct PET LORs along the IEC-Y axis, IEC-X axis, or IEC-Z axis. The effect of shuffling the time stamps of deblurred PET LORs is conceptually depicted in FIGS. 10D-10F. FIG. 10D depicts LTS images reconstructed from PET LORs that have not been motion-corrected, where the motion trajectory is retained. FIG. 10E depicts LTS images reconstructed from PET LORs that have been motion-corrected, but the motion-correction is not complete, so while the motion trajectory is suppressed, it is not entirely removed. FIG. 10F depicts LTS images reconstructed from PET LORs that have been motion-corrected and their time stamps shuffled. The motion component is completely removed and the LTS images are “static”, but because the time stamps are shuffled, they are blurred, similar to the LTS images depicted in FIG. 10C. However, in comparison to FIG. 10C, the LTS images in FIG. 10F are less blurry. Generating an error metric and/or uncertainty model using these LTS images may further reduce the impact of correlated motion errors on deblurring methods and location estimator methods. Simulated imaging data may be generated using any combination of the methods described herein. For example, generating simulated imaging data may comprise shuffling the time stamps of PET LORs, and then adding in any desired motion pattern, such as restoring the original motion pattern (using circshift) and/or applying simulated motion patterns.

Generating 1014 a tracking uncertainty model from the set of estimated target shifts may be performed using any of the methods described above. For example, the uncertainty model may comprise an uncertainty cloud where the points in the cloud include the set of estimated target shifts. Alternatively, or additionally, the points in the uncertainty cloud may include the differences between the estimated target region shifts and the target region shifts determined from the deblurred PET imaging data (e.g., the method described above and depicted in FIG. 8B).

Another method for generating a tracking error metric and/or location uncertainty model may not include deblurring the PET imaging data using the methods described herein. Instead, these methods of generating a location uncertainty model may comprise iteratively training/updating the location estimation methods on the planning imaging data, and at each iteration, incrementally correcting the motion artifacts in the planning imaging data. One variation of a method for iteratively correcting the motion artifacts may comprise training the one or more location estimation methods on non-corrected (i.e., “blurred”) planning PET LTS images, applying the trained location estimation methods on the planning PET LTS images to calculate the estimated shift for each of the LTS images, and adjusting the planning PET LTS images by the calculated estimated shift for each LTS image. Optionally, this method may be iterated (i.e., repeated) as many times as may be desired. For example, in the second iteration, the location estimation methods may be trained on the adjusted (i.e., motion-compensated, or motion-subtracted) PET LTS images from the previous iteration, applying the re-trained location estimation methods on the original non-corrected planning PET LTS images to calculate a second estimated shift for each of the LTS images, and adjusting the planning PET LTS images by the calculated second estimated shift for each LTS image. Adjusting the LTS images by the calculated shift may include applying the inverse shift value to each LTS image to “move” the LTS image to a reference location, e.g., to align the LTS image to the reference location. At the conclusion of the desired number of iterations, the original planning PET LTS images may be motion-corrected, e.g., deblurred, and optionally, the target region shift for each motion phase or shift may be calculated. This motion-corrected imaging data may be used for treatment planning or for any desired purpose, including generating an uncertainty model. Generating an uncertainty model using PET LTS images that have been iteratively motion-corrected may comprise reassigning detection time stamps of the PET LORs (that were reconstructed to obtain the original/first set of PET LTS images) to different LORs, reconstructing a second set of PET LTS images from the LORs with reassigned detection time stamps, applying one or more location estimator methods to the second set of PET LTS images to determine a set of estimated target shifts, and generating a tracking uncertainty model (e.g., uncertainty cloud) from the set of target shifts.

Some variations of treatment planning and/or radiation delivery methods (which may also be referred to as “workflows”) may comprise evaluating the quality and/or performance of the image deblurring process and/or the location uncertainty model. This evaluation may help a clinician determine whether satisfactory location estimation is possible for a patient. In some scenarios, the PET imaging data may be insufficient for precise location estimation of the target region, and/or the PET tracer uptake may not be well-localized to the target regions (i.e., non-target regions have significant PET tracer uptake) so that the PET signal intensity in the background is similar to, or even exceeds, the PET signal intensity in the target region. A clinician may decide that BgRT is not appropriate in such scenarios. Some variations of treatment planning and/or treatment delivery workflows may include one or more safety checks that include evaluating one or more of: the PET signal in the target region(s), PET signal in the background and/or OARs, quality of the deblurring process and/or characteristics of the location uncertainty model. During treatment planning, the method may comprise determining whether a certain number of conditions are met before proceeding with generating a treatment plan (e.g., fluence map optimization). In one example, a treatment planning method may comprise determining whether one or more of the following three conditions are met: (1) the PET deblurring ratio, defined as the ratio of the activity concentration (AC, having units of kBq/mL) of the target region in the deblurred PET image to the AC of the target region in the motion averaged (e.g., not motion-corrected) PET image, must be greater than or equal to a predetermined threshold. (2) The estimated size or spread of the uncertainty model must be less than or equal to a threshold distance along any dimension. (3) The estimated mean of the uncertainty model must be no more than a threshold distance away from the target region centroid along any dimension. If one or more of these conditions (or other alternative and/or additional conditions) are not met, an error message may be generated and output to a display device, and in some variations, the system may not allow the user to proceed with treatment plan optimization. The AC value of a target region in a PET image may be calculated from the pixels/voxels enclosed by the contours (i.e., boundaries) of the target region. In some variations, the threshold value of the PET deblurring ratio may be approximately 0.9, since a value less than 0.9 may indicate that the deblurring process has failed. In some variations, the spread of an uncertainty model may be defined as 2 times the standard deviation of the uncertainty model, and the threshold may require that the spread is less than or equal to 20 mm along any dimension. In some variations, the estimated mean of an uncertainty model may be the magnitude of a target shift (e.g., each point in an uncertainty cloud may have a value indicating a shift away from the “true” location of the target) and the threshold may require that the shift must be less than 3 mm away from the target centroid along any dimension. Alternatively, or additionally, a target-to-background (TBR) metric may be evaluated. The TBR may be calculated during treatment planning and/or treatment delivery. In one variation, the TBR may be computed from a planning or prescan PET image as

TBR=AC^{Target}/AC^{Background}

where AC^{Target} and AC^{Background} are the same values used to calculate AC as described above. In some variations, if the TBR is not within an acceptable range, treatment planning and/or treatment delivery may not proceed. Optionally, some methods for evaluating PET images/imaging data may comprise calculating and evaluating whether one or more characteristic(s) of a PET image is within an acceptable operating range. One characteristic of a PET image may be a contrast of the PET signal within a target region (e.g., PTV contour) against the background PET signal (e.g., within a BTZ contour). For example, the contrast ratio may be calculated as the difference between the mean PET signal within the target region contour and the mean PET signal in the BTZ (excluding the PET signal in the target region) divided by the mean PET signal within the target region contour. An acceptable operating range for the contrast ratio may be threshold ratio value. The threshold ratio value may be 0.4 or more (e.g., for treatment planning) or may be 0.8 or more (for treatment delivery). The threshold ratio value may be determined or selected by a user or clinic, based on their preference and clinical risk tolerance. Checking the operating range of a PET image may help ensure that the calculation of AC and NTS, along with the other quality evaluations and/or metrics are reliable. Operating range checks may help guard against PET images with inadequate count statistics both at planning (during plan optimization) as well as during radiation delivery using the prescan PET images. In one variation, operating range is calculated using the clinical PTV, GTV, and BTZ from the treatment plan. If a PET image(s) is not within the operating range, the treatment planning system and/or radiotherapy system may generate a notification indicating that the PET is out of range and it may not be safe to continue with planning and/or radiotherapy. In some variations, a controller may generate an interlock that does not allow the user to move forward with further treatment planning and/or delivery steps.

One variation of radiotherapy treatment planning may comprise initializing the one or more location estimation methods using the planning PET images and/or PET imaging data, evaluating the quality of the location estimation method(s) by evaluating characteristics of the corresponding location uncertainty model, and if the location estimation method(s) are of sufficient quality (e.g., able to estimate the target region location with reasonable certainty and precision), proceed to generating a radiotherapy treatment plan. For PET-guided or biology-guided radiotherapy, a radiotherapy treatment plan may comprise a planned fluence map that provides the desired dose to the target region and a set of firing filters that are to be combined with an anchor point location calculated from real-time imaging data to calculate a radiation fluence for delivery. In one variation, initializing location estimation method(s) may comprise calculating a template or filter for use in an ACF location estimation method, calculating probability density functions for use in an MLC location estimation method, and setting parameters of a Kalman filter. These may be computed by a controller using any of the methods described herein. Initialization of location estimation methods may optionally comprise generating a location uncertainty model by applying the initialized location estimation methods to PET imaging data, for example, planning PET images and/or imaging data to compute a set of most-likely locations of the target region. For example, the location uncertainty model may comprise an uncertainty cloud that includes the set of locations of the target region as estimated by the location estimation methods. These computed most-likely locations may be compared to “ground truth” locations of the target region and the difference in the locations may be included in the location uncertainty model. The “ground truth” locations of the target region may be calculated by deblurring the planning PET images using the methods described herein. In some variations of treatment planning, evaluating the characteristics of the location uncertainty model may include one or more of evaluating the performance of the deblurring process, and evaluating one or more characteristics of the location uncertainty model. Examples of location uncertainty model characteristics that may be evaluated may include the PET deblurring ratio, an estimated spread of the location uncertainty model, and a mean of the location uncertainty model, as described above (FIGS. 7B-7C). Without wishing to be bound by therapy, evaluating the quality of the location uncertainty model may provide insight as to whether effective location tracking is possible for the target region. In some variations, the process of initializing the location estimation methods may generate one or more ACF templates and/or filters, MLE probability density functions, a location uncertainty model for the location estimation method (e.g., any of the location estimation methods or algorithms described herein), and/or motion-corrected (e.g., deblurred) PET images. After initializing the location estimation algorithm(s), some treatment planning methods may comprise aligning the planning PET images to an anatomical image where one or more contours (e.g., PTV, GTV, etc.) have been defined. Alignment may comprise shifting the planning PET images (which may be motion-corrected) so that the PET-avid target region may be registered with the desired contour and/or structure in the anatomical image (e.g., CT image, MR image). For example, aligning a planning PET image may comprise aligning a centroid of the target region in the motion-corrected planning PET image with the centroid of a GTV contour in a CT image.

Similar evaluations may be performed at the start of a treatment session. For example, in a BgRT treatment session, a PET prescan imaging acquisition may be performed before radiation delivery. The acquired PET imaging data may comprise PET LOR data that may be reconstructed into a series of PET LTS images, for example, using TOF-FBP (e.g., SSRB-FBP). This series of LTS images may be deblurred using any of the deblur methods described herein, and then the prescan uncertainty model may be constructed using the one or more trained location estimators (e.g., ACF and MLE location estimation methods) calculated during treatment planning. Finally, the prescan uncertainly model may be masked by the BTZ and reprojected to form a beam's eye view prescan uncertainty model projection volume that may then be used with the firing filters of the firing matrix (P) and the beamlet dose matrix (A) from the treatment plan to predict the expected dose distribution to be delivered based on the prescan PET imaging data. The DVH curves from the predicted prescan dose may then be compared to a bounded DVH (bDVH) from the approved treatment plan to evaluate a bDVH pass criterion. This criterion may comprise the percentage of points in the prescan DVH(s) that fall within the bDVH for all structures other than the target region (e.g., GTV, PTV) and dose shaping structures. The evaluation is performed on points with dose greater than 10% of the prescription dose and it passes if the percentage of points meeting the criteria is greater than or equal to 95%. Prior to calculating the predicted prescan dose, some delivery workflows may comprise evaluating the deblurring performance and the quality of the prescan uncertainty model. In some variations, the prescan PET image deblurring ratio (i.e., the ratio of AC calculated from deblurred PET to AC calculated from motion averaged PET) must exceed a certain threshold, e.g., be greater than or equal to 0.9. Additionally, the difference between the planned and prescan location uncertainty model spread (i.e., 2 times the standard deviation) must be less than 3 mm along all dimensions and the difference between the plan and prescan uncertainty model relative mean (i.e., absolute model mean-detected motion mean) must be less than 3 mm along all dimensions. If any of these three criteria are not met, PET evaluation fails. Some methods may comprise generating a notification to the user that includes a GUI that reports the status of these criteria and a user prompt to exit delivery and/or take alternative actions.

If PET evaluation is successful (i.e., all pass criteria are satisfied) the system calculates an initial delivery normalization factor (k1) that is used later by the SCINTIX delivery algorithm. This initial delivery normalization factor accounts for activity variations between the functional modeling PET and the prescan PET.

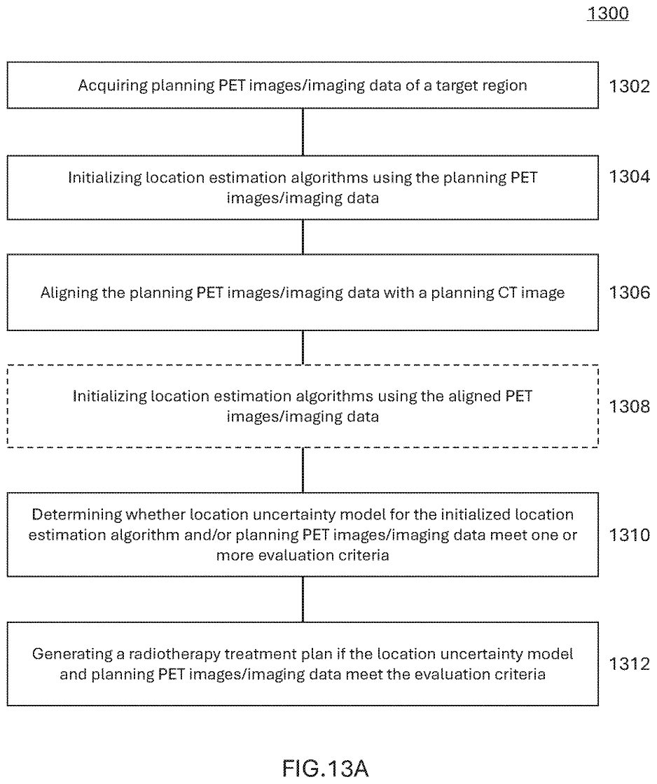

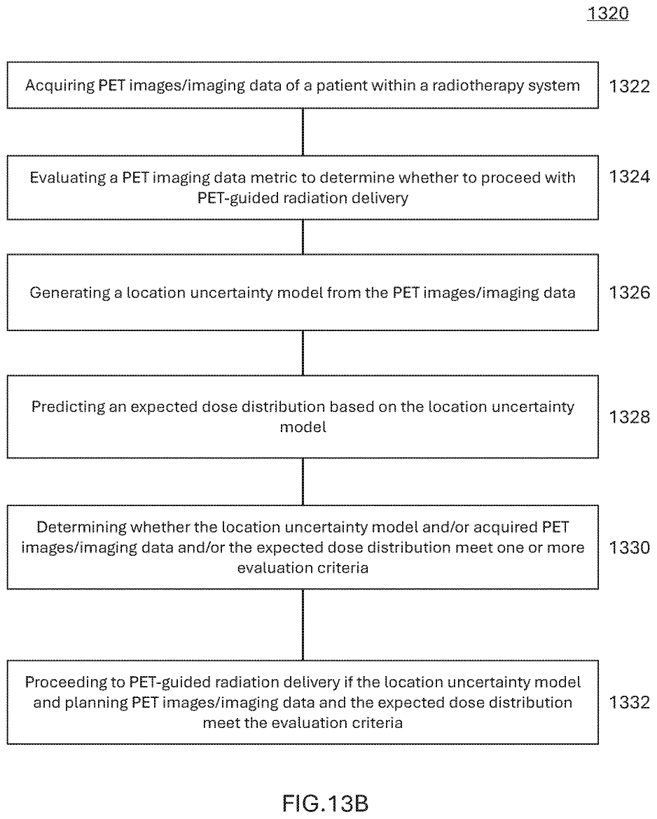

FIG. 13A is a flowchart depicting one variation of a method or workflow for radiotherapy treatment planning for biology-guided radiotherapy using PET imaging data. Method 1300 may comprise acquiring 1302 planning PET images/imaging data of a target region, initializing 1304 location estimation algorithms using the planning PET images/imaging data, aligning 1306 the planning PET images/imaging data with a planning CT image, determining 1310 whether location uncertainty model for the initialized location estimation algorithm and/or planning PET images/imaging data meet one or more evaluation criteria, and generating 1312 a radiotherapy treatment plan if the location uncertainty model and planning PET images/imaging data meet the evaluation criteria. Optionally, method 1300 may comprise initializing 1308 location estimation algorithms using the aligned PET images/imaging data. The steps in method 1300 may use any of the methods described herein for image acquisition, location estimation algorithm initialization, PET image alignment, location uncertainty model generation, quality evaluation, and/or generating a treatment plan (e.g., firing filters, planned radiation fluence map, planned dose distribution, etc.). In some variations, the radiotherapy treatment plan may comprise a planned fluence map and firing filters that convert anchor point images into delivery fluences. For example, the one or more evaluation criteria computed in step 1310 may comprise one or more of the PET deblur ratio, location uncertainty model spread, location uncertainty model mean/average, activity concentration, normalized target signal, operating range, tumor to background ratio, as described above. The radiotherapy treatment plan may comprise a set of firing filters that may be computed by an iterative optimization method described above. Method 1300 may further comprise computing the dose distribution of the planned fluence map and confirm that the dose distribution has characteristics that are within an acceptable range defined by a clinician. For example, method 1300 may comprise calculating a DVH from the planned fluence map, comparing the DVH to a bounded DVH, and computing the proportion of points on the calculated DVH that fall outside the bounded DVH. If the proportion of points that fall outside the bounded DVH exceed a threshold (e.g., more than 5% of the points fall outside the bounded DVH), a notification may be generated to inform the user that the planned dose distribution deviates from an acceptable dose range.

FIG. 13B is a flowchart depicting one variation of a safety method or workflow for radiotherapy treatment delivery for biology-guided radiotherapy. Method 1302 may be used in conjunction with the radiation delivery methods described above (e.g., FIGS. 2A-2C, as well as any of the individual location estimation methods disclosed herein). Method 1320 may comprise acquiring 1322 PET prescan images/imaging data of a patient within a radiotherapy system, evaluating 1324 a PET imaging data metric to determine whether to proceed with PET-guided radiation delivery, generating 1326 a location uncertainty model from the PET images/imaging data, predicting 1328 an expected dose distribution based on the location uncertainty model, determining 1330 whether the location uncertainty model and/or acquired PET images/imaging data and/or the expected dose distribution meet one or more evaluation criteria, and proceeding to PET-guided radiation delivery 1332 if the location uncertainty model and planning PET images/imaging data and the expected dose distribution meet the evaluation criteria. Evaluating 132 a PET imaging data metric may comprise evaluating multiple metrics, including the activity concentration, normalized target signal, one or more operating range metrics, and/or target-to-background ratio. Ensuring that these metrics are within an acceptable range on the day of treatment may help to ensure that the radiotherapy system delivers the prescribed dose(s) to the target region(s) while adhering to the radiation limitations for surrounding tissue. For example, assessing the evaluation criteria may help determine whether effective target location tracking possible (e.g., characterized by the PET deblur ratio, the difference between the planned location uncertainty model and the PET prescan uncertainty model, such as differences in their spread and/or mean/average), whether there is sufficient PET signal and/or target region contrast in the BTZ for target tracking (e.g., characterized by the activity concentration and/or normalized target signal), and whether the predicted dose will fall within the bounds of an approved bDVH. These assessments and evaluations may cumulatively be referred to as “PET evaluation”. The outcome of these assessments may be provided in a GUI that is output to a display for the user to review.

Generating 1326 a location uncertainty model may use any of the methods described herein (e.g., FIG. 9B), and may comprise deblurring the PET prescan images/imaging data, initializing the location estimation methods using the PET prescan images (e.g., deblurred PET prescan images), applying the location estimation methods to the PET prescan images/imaging data to generate location estimates, and evaluating the effectiveness of the PET deblur (e.g., computing and evaluating the PET deblur ratio), computing and evaluating the spread of the location uncertainty model, and/or computing and evaluating the mean/average value of the location uncertainty model. In some variations, predicting 1328 an expected dose distribution may comprise masking the location uncertainty model generated from the PET prescan images (i.e., the prescan location uncertainty model) using the BTZ contour, reprojecting the prescan location uncertainty model to form a beam's eye view prescan location uncertainty model, convolving the beam's eye view prescan location uncertainty model with firing filters from the treatment plan to attain a predicted fluence map, and computing the expected dose distribution by combining the predicted fluence map with a beamlet dose matrix calculated during treatment planning. Some variations may comprise generating DVH curves from the predicted dose distribution and comparing the DVH curves to the bounded DVH (bDVH) from the approved treatment plan to evaluate the bDVH pass criterion. This criterion may be based on the percentage of points in the prescan DVHs that fall within the bDVH for all structures other than the GTV, PTV and dose shaping structures. The evaluation may be performed on points with dose greater than 10% of the prescription dose and the evaluation is considered as PASSED if the percentage of points meeting the criteria is greater than or equal to 95%. While method 1320 may be used to assess the safety and quality of the radiotherapy delivery to a patient in a treatment session, one or more of the assessments in method 1320 and described above may be used as part of treatment planning to confirm that a patient is eligible for PET-guided radiotherapy.

While the examples and methods described herein are in the context of radiotherapy that uses PET imaging data (e.g., biology-guided radiotherapy), it should be understood that the same methods may be used for radiotherapy involving imaging data of other modalities, including, but not limited to CT imaging data, MR imaging data, optical imaging data, and/or ultrasound imaging data. Moreover, although the examples provided herein are described in the context of a radiotherapy treatment session, it should be understood that the systems and methods may be used during a non-treatment session, i.e., in the absence of a patient. For example, any of the systems and methods described herein may be used in a quality assurance (QA) and/or calibration session with a phantom in the treatment area instead of a patient. In some variations, the phantom may comprise one or more target regions that have positron emission activity that may mimic the uptake of PET tracer in a patient. Alternatively, or additionally, a radiation measurement device may be positioned in the treatment area. A treatment plan may be delivered and the phantom and/or radiation measurement device may be used to evaluate whether the radiotherapy system functioned properly to deliver the treatment plan. In the case of biology-guided radiotherapy, the treatment plan may include firing filters and an adaptive correlation filter and/or probability density function and/or anchor or landmark (e.g., tumor centroid) for each target region, and the methods described herein may be used to generate a radiation fluence for delivery during a QA and/or calibration session.

While various inventive variations and embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described variations and embodiments can be implemented in one or more desired ways. For example, embodiments of designing and making the technology disclosed herein may be implemented using hardware, software or a combination thereof. When implemented in software, the software code can be executed on any suitable processor or collection of processors, whether provided in a single computer (e.g., controller) or distributed among multiple computers (e.g., controllers). Any of the methods disclosed herein may be implemented in machine-readable code and executed using any one or combination of the controllers and/or processors disclosed herein. The methods may be performed solely by controller(s) and/or processor(s). Alternatively, or additionally, the methods may be performed by a user in conjunction with one or more controller(s) and/or processor(s).

Further, it should be appreciated that a computer or controller may be embodied in any of a number of forms, such as a rack-mounted computer, a desktop computer, a laptop computer, or a tablet computer. Additionally, a computer or controller may be embedded in a device not generally regarded as a computer but with suitable processing capabilities, including a Personal Digital Assistant (PDA), a smart phone or any other suitable portable or fixed electronic device.

Also, a computer or controller may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers or controllers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of embodiments as discussed above. Any of the algorithms disclosed herein may be written as computer code or computer-executable instructions and implemented as a program or software. Additionally, it should be appreciated that according to one aspect, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys a relationship between the fields. However, any suitable mechanism may be used to establish a relationship between information in fields of a data structure, including through the use of pointers, tags or other mechanisms that establish relationship between data elements.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of improving radiation dose conformality to a target region, the method comprising:
acquiring imaging data of a target region using image detectors;
analyzing the imaging data using a first algorithm to generate a first estimated location of the target region;
analyzing the imaging data using a second algorithm to generate a second estimated location of the target region, wherein the second algorithm is different from the first algorithm;
generating a final location of the target region by combining the first estimated location and the second estimated location; and
calculating a radiation fluence according to the final location of the target region, wherein the calculated radiation fluence, when emitted, delivers a conformal radiation dose to the target region.

2. The method of claim 1, wherein generating the final location of the target region comprises combining the first estimated location and the second estimated location using a Kalman filter.

3. The method of claim 1, further comprising analyzing the imaging data using a third algorithm to generate a third estimated location of the target region, and wherein generating the final location of the target region comprises combining the first estimated location, the second estimated location, and the third estimated location.

4. The method of claim 3, wherein generating the final location of the target region comprises combining the first estimated location, the second estimated location, and the third estimated location using a Kalman filter.

5. The method of claim 1, wherein analyzing the imaging data using the first algorithm further comprises calculating a first confidence value associated with the first estimated location, and analyzing the imaging data using the second algorithm further comprises calculating a second confidence value associated with the second estimated location, and wherein generating the final location of the target region comprises weighting the first estimated location by the first confidence value, weighting the second estimated location by the second confidence value, and combining the weighted first estimated location and the weighted second estimated location.

6. The method of claim 1, wherein using the first algorithm comprises applying a correlation filter to the imaging data to generate the first estimated location of the target region and wherein using the second algorithm comprises performing a maximum likelihood estimation analysis on the imaging data to generate the second estimated location of the target region.

7. The method of claim 1, wherein using the first algorithm comprises applying a correlation filter to the imaging data to generate the first estimated location of the target region and wherein using the second algorithm comprises calculating a location of a centroid of the imaging data to generate the second estimated location of the target region.

8. The method of claim 6 or claim 7, wherein the correlation filter is an adaptive correlation filter that is trained on images of the target region and locations of the target region in the images.

9. The method of claim 1, wherein using the first algorithm comprises performing a maximum likelihood estimation analysis on the imaging data to generate the first estimated location of the target region and wherein using the second algorithm comprises calculating a location of a centroid of the imaging data to generate the second estimated location of the target region.

10. The method of claim 1, wherein the imaging data comprises time-of-flight PET imaging data, and wherein the first algorithm comprises calculating a location of a centroid using the time-of-flight PET imaging data to generate the first estimated location of the target region.

11. The method of claim 1, further comprising generating a notification that indicates the final location of the target region.

12. The method of claim 1, wherein the imaging data comprises positron annihilation emission path data, and the image detectors comprise PET detectors.

13. The method of claim 1, wherein the imaging data comprises time-of-flight (TOF) positron annihilation emission path data.

14. The method of claim 13, wherein using the first algorithm comprises calculating a location of a centroid of the TOF positron annihilation emission path data by determining a region where the TOF positron annihilation emission paths intersect.

15. The method of claim 1, wherein acquiring imaging data comprises acquiring imaging data from the image detectors for a first time duration, and wherein the first algorithm generates the first estimated location using the imaging data acquired during a second time duration that is a subset within the first time duration and the second algorithm generates the second estimated location using the imaging data acquired during the first time duration.

16. The method of claim 15, wherein the first time duration is from about 3 seconds to about 6 seconds, and the second time duration is from about 200 ms to about 600 ms.

17. The method of claim 1, wherein combining the first estimated location and the second estimated location comprises correcting a bias of the first estimated location using the second estimated location.

18. The method of claim 1, wherein combining the first estimated location and the second estimated location comprises correcting a bias of the second estimated location using the first estimated location.

19. The method of claim 15, wherein the second estimated location is a running average of the target region location during the first time duration, and the first estimated location is the location of the target region during the second time interval.

20. The method of claim 1, wherein the final location of the target region includes spatial coordinates of the target region.

21. The method of claim 20, wherein the spatial coordinates comprise 3-D coordinates.

22. The method of claim 1, wherein calculating the radiation fluence for delivery comprises adjusting a planned fluence map according to the final location of the target region.

23. The method of claim 22, wherein adjusting the planned fluence map comprises shifting the planned fluence map to the final location of the target region.

24. The method of claim 1, wherein calculating the radiation fluence for delivery comprises generating an image of the final location of the target region and convolving the generated image of the final location with a firing filter.

25. The method of claim 1, wherein acquiring the imaging data of the target region and calculating the radiation fluence for delivery both occur in a single radiation delivery session.

26. The method of claim 1, wherein acquiring the imaging data of the target region and calculating the radiation fluence for delivery both occur in real-time.

27. The method of claim 1, wherein calculating the radiation fluence for delivery occurs within 1 second of acquiring the imaging data of the target region.

28. The method of claim 27, wherein calculating the radiation fluence for delivery occurs within 500 ms of acquiring the imaging data of the target region.

29. The method of claim 1, further comprising delivering the calculated radiation fluence to the final location of the target region.

30. A maximum likelihood estimation method for determining the location of a target region using images having a low-photon count, the method comprising:
acquiring a low-photon count image of a target region, where the target region has an initial location;
applying a plurality of probability density functions corresponding to a plurality of target region shifts to the low-photon count image to calculate a likelihood value for each of the plurality of probability density functions;
determining a target region shift by identifying the target region shift that corresponds to a maximum likelihood value from the likelihood values for the plurality of probability functions; and
calculating an updated location of the target region by applying the determined target region shift to the initial location of the target region.

31. The method of claim 30, wherein the plurality of probability density functions is generated by shifting a nominal probability density function by the plurality of target region shifts.

32. The method of claim 31, wherein the nominal probability density function is derived from a high-photon count image of the target region location.

33. The method of claim 32, wherein the high-photon count image of the target region comprises positron annihilation emission path imaging data acquired during treatment planning.

34. The method of claim 32, wherein the high-photon count image of the target region comprises positron annihilation emission path imaging data acquired during a treatment session.

35. The method of claim 32, wherein the high-photon count image of the target region comprises positron annihilation emission path imaging data acquired during a radiation delivery session.

36. The method of claim 30, wherein the low-photon count images comprise positron annihilation emission path data acquired during an acquisition time interval that is less than 10 seconds.

37. The method of claim 30, further comprising generating an additional probability density function for a previously-determined target region shift.

38. The method of claim 37, wherein generating the additional probability density function comprises applying a Bayesian model to the updated target location that has been shifted by a previously-determined target region shift.

39. The method of claim 30, wherein the low-photon count image comprises lines-of-response (LORs) data measured by PET detectors.

40. A method for generating a reduced-motion PET image of a target region for radiotherapy treatment planning, the method comprising:
decomposing PET imaging data of a target region into a set of principal components using principal component analysis;
identifying a principal component from the set of principal components that has a dominant frequency similar to a predetermined frequency range;
defining a set of motion phases using the identified principal component;
grouping the PET imaging data into its corresponding motion phase within the set of motion phases;
generating phase-gated PET images by summing the PET imaging data at each motion phase;
aligning each of the phase-gated PET images with a reference target location;
summing the phase-gated PET images to generate a reduced-motion image of the target region; and
generating a radiotherapy treatment plan using the reduced-motion image of the target region.

41. The method of claim 40, wherein the radiotherapy treatment plan comprises a planned radiation fluence map to deliver a prescribed radiation dose to the target region.

42. The method of claim 40, wherein aligning each of the phase-gated PET images with the reference target location comprises determining a shift vector for each of the phase-gated PET images by comparing the phase-gated PET images with the reference target region location, and shifting each of the phase-gated PET images according to the shift vector.

43. The method of claim 42, wherein the shift vector is a 3-D shift vector.

44. The method of claim 40, wherein the set of principal components comprises 3 or more principal components.

45. The method of claim 40, further comprising acquiring PET imaging data of the target region using PET detectors.

46. The method of claim 40, wherein the predetermined frequency range is approximately a frequency range of human respiration.

47. The method of claim 40, wherein the predetermined frequency range is approximately a frequency range of human peristaltic motion.

48. The method of claim 40, wherein the predetermined frequency range is approximately a frequency range of about 0.05 Hz to about 0.6 Hz.

49. The method of claim 40, further comprising applying a difference of Gaussians filter to the PET imaging data before decomposing the PET imaging data into the set of principal components.

50. The method of claim 40, wherein the PET imaging data comprises lines-of-response data.

51. The method of claim 50, wherein the lines-of-response data is acquired using PET detectors within a specified acquisition time interval.

52. The method of claim 51, wherein the specified acquisition time interval is 5 seconds or less.

53. The method of claim 52, wherein the specified acquisition time interval is 1 second or less.

54. The method of claim 52, wherein the specified acquisition time interval is 500 ms or less.

55. The method of claim 40, wherein the PET imaging data comprises time-of-flight PET data.

56. The method of claim 40, wherein identifying the principal component that has a dominant frequency similar to the predetermined frequency range comprises applying a Fourier transform to the set of principal components.

57. The method of claim 40, wherein defining the set of motion phases comprises applying a Hilbert transform to the identified principal component.

58. The method of claim 40, wherein the set of motion phases comprises 16 phases.

59. The method of claim 40, wherein the method further comprises determining a phase-specific location of the target region for each phase of the set of motion phases based on the phase-gated PET images, and calculating a motion vector for each group of PET imaging data based on the phase-specific location of the target region in the set of motion phases.

60. A method for generating a PET image training data set for testing target tracking methods, the method comprising:
decomposing PET imaging data of a target region into a set of principal components using principal component analysis;
identifying a principal component from the set of principal components that has a dominant frequency similar to a predetermined frequency range;
defining a set of motion phases using the identified principal component;
grouping the PET imaging data into its corresponding motion phase within the set of motion phases;
generating phase-gated PET images by summing the PET imaging data at each motion phase;
determining a target region location for each motion phase by comparing the phase-gated PET images to a reference target location; and
generating a training data set comprising the PET imaging data for each motion phase and the determined target region location associated with that motion phase.

61. The method of claim 60, wherein the PET imaging data comprises PET lines-of-response (LOR) data, and the training data set comprises PET LOR data for each motion phase and the determined target region location associated with that motion phase.

62. The method of claim 60, wherein the PET imaging data comprises PET imaging data acquired during a series of acquisition time intervals, and the training data set comprises PET imaging data acquired during specific acquisition time intervals within a motion phase and the determined target region location associated with that motion phase.

63. The method of claim 60, wherein the set of principal components comprises 3 or more principal components.

64. The method of claim 60, further comprising acquiring PET imaging data of the target region using PET detectors.

65. The method of claim 60, wherein the predetermined frequency range is approximately a frequency range of human respiration.

66. The method of claim 60, wherein the predetermined frequency range is approximately a frequency range of human peristaltic motion.

67. The method of claim 60, wherein the predetermined frequency range is approximately a frequency range of about 0.05 Hz to about 0.6 Hz.

68. The method of claim 60, wherein the PET imaging data comprises lines-of-response data.

69. The method of claim 38, wherein the lines-of-response data is acquired using PET detectors within a specified acquisition time interval.

70. The method of claim 39, wherein the specified acquisition time interval is 5 seconds or less.

71. The method of claim 70, wherein the specified acquisition time interval is 1 second or less.

72. The method of claim 70, wherein the specified acquisition time interval is 500 ms or less.

73. The method of claim 60, wherein the PET imaging data comprises time-of-flight PET data.

74. The method of claim 60, wherein identifying the principal component that has a dominant frequency similar to the predetermined frequency range comprises applying a Fourier transform to the set of principal components.

75. The method of claim 60, wherein defining the set of motion phases comprises applying a Hilbert transform to the identified principal component.

76. The method of claim 60, wherein the set of motion phases comprises 16 phases.

77. A method for generating a reduced-motion PET image of a tumor for radiotherapy treatment planning, the method comprising:
calculating a background level of PET activity in a first PET image of a tumor, wherein the tumor has a tumor contour and an initial level of PET activity within the tumor contour;
defining a set of PET activity isocontours in the first PET image within a predefined contour that encompasses the tumor, wherein the isocontours delineate a corresponding set of isocontour shells;
calculating a tumor PET activity for each isocontour shell;
calculating a total tumor PET activity within the predefined contour by summing the tumor PET activity in the set of isocontour shells;
generating a background image by setting a PET activity for the set of isocontour shells to a background level of PET activity;
adjusting the initial level of PET activity within the tumor contour to have the calculated total tumor PET activity;
generating a second PET image by combining the tumor contour with the adjusted level of PET activity and the background image; and
generating a radiotherapy treatment plan using the second PET image.

78. The method of 77, wherein the predefined contour is a biology-tracking zone (BTZ) contour.

79. The method of 77, further comprising calculating the background level of PET activity by calculating PET activity in a region outside of the predefined contour.

80. The method of 79, wherein the region outside of the predefined contour is a shell region that is an expansion of the predefined contour.

81. The method of claim 77, further comprising calculating the background level of PET activity by subtracting the calculated tumor PET activity for an isocontour shell from the PET activity for the isocontour shell.

82. The method of claim 77, wherein adjusting the initial level of PET activity within the tumor contour comprises scaling the initial level of PET activity such that a total PET activity within the tumor contour matches the calculated total tumor PET activity.

83. The method of claim 77, further comprising calculating a scaling factor based on a ratio of the calculated total tumor PET activity to the initial level of PET activity, and wherein adjusting the initial level of PET activity comprises calculating a PET activity offset by multiplying pixel values within the tumor contour by the scaling factor and/or modulating the pixel values within the tumor contour by the PET activity offset.

84. The method of claim 77, wherein adjusting the initial level of PET activity within the tumor contour comprises normalizing the initial level of PET activity within the tumor contour to the total tumor PET activity.

85. The method of claim 77, further comprising acquiring a CT image of the tumor and wherein the tumor contour is defined using the CT image.

86. The method of claim 85, wherein the initial level of PET activity within the tumor contour is derived by overlaying the CT image over the first PET image and calculating PET activity within the tumor contour.

87. The method of claim 77, further comprising determining an isocontour PET activity for each isocontour shell based on pixel intensity values of the blurred PET image, and wherein calculating the tumor PET activity for each isocontour shell comprises subtracting the background level of PET activity from the isocontour PET activity.

88. The method of claim 77, further comprising determining an isocontour PET activity for each isocontour shell based on pixel intensity values of the blurred PET image, and wherein calculating the tumor PET activity for each isocontour shell comprises scaling the isocontour PET activity by a factor derived from a ratio of the background level of PET activity to a PET activity of the isocontour.

89. A method for radiotherapy treatment planning, the method comprising:
acquiring PET images of a target region;
deriving a first set of target region locations corresponding to phases of a target region motion trajectory from the acquired PET images;
deriving a second set of target region locations corresponding to phases of the target region motion trajectory by analyzing the acquired PET images using a location estimation algorithm;
generating a location uncertainty model by determining differences between the first and second sets of target region locations for each phase of the target region motion trajectory; and
generating a radiotherapy treatment plan comprising a set of firing filters iteratively calculated from the uncertainty model.

90. The method of claim 89, further comprising deblurring the acquired PET images to generate motion-corrected PET images and defining the phases of the target region motion trajectory using the motion-corrected PET images.

91. The method of claim 90, wherein deriving the first set of target region locations comprises aligning the motion-corrected PET images to a reference location of the target region.

92. The method of claim 90, further comprising training the location estimation algorithm on the motion-corrected PET images.

93. The method of claim 90, wherein deblurring the acquired PET images comprises decomposing the PET images into a set of principal components and identifying a principal component from the set of principal components, and wherein defining the phases of the target region motion trajectory comprises defining the phases based on the identified principal component.

94. The method of claim 89, wherein the location uncertainty model comprises a location uncertainty cloud comprising a plurality of target region locations.

95. The method of claim 89, wherein the location uncertainty model comprises a location uncertainty cloud comprising a plurality of target region shifts from an initial target region location.

96. The method of claim 94, wherein generating the radiotherapy treatment plan comprises iterating through candidate firing filters such that when convolved with the location uncertainty model, the radiotherapy treatment plan attains a prescribed radiation dose to the target region while minimizing a dose cost function.

97. The method of claim 89, wherein acquiring PET images of a target region comprises acquiring PET images concurrently with acquiring target region position data using a position sensor, and wherein deriving the first set of target region locations comprises defining phases of the motion trajectory using the target region position data and binning the acquired PET images into the corresponding phase during which the PET images were acquired.

98. A method for generating a tracking uncertainty model, the method comprising:
acquiring PET images of a target region over a plurality of motion phases;
deblurring the target region images to obtain motion-corrected PET images;
training a location estimation algorithm to identify a location of a target region in an image;
applying the location estimation algorithm to the motion-corrected PET images to determine a set of estimated target region shifts; and
generating a tracking uncertainty model from the set of estimated target region shifts.

99. The method of claim 98, wherein training the location estimation algorithm comprises using the motion-corrected PET images that have been aligned to a reference target region location.

100. The method of claim 99, wherein the set of estimated target region shifts is a first set of estimated target region shifts, and the method further comprises applying the location estimation algorithm to the acquired PET images to determine a second set of estimated target region shifts, and wherein generating the tracking uncertainty model comprises computing differences between the first set of estimated target region shifts and the second set of estimated target region shifts.

101. The method of claim 98, wherein training the location estimation algorithm comprises using the acquired PET images.

102. A method for evaluating the quality of a location estimation algorithm, the method comprising:
generating a first series of target region location data for multiple motion phases based on images of a moving target region;
generating a second series of estimated target region location data for the multiple motion phases by applying a location estimation algorithm to the images of a moving target region;
generating a location uncertainty model by comparing the first series of target region location data with the second series of target region location data;
computing a characteristic of the location uncertainty model;
evaluating the characteristic of the location uncertainty model against a predetermined acceptable range of that characteristic; and
determining a quality of the location estimation algorithm based on whether the evaluated characteristic is within the acceptable range.

103. The method of claim 102, wherein the location uncertainty model has a spread defined as 2 times a standard deviation of the location uncertainty model, and wherein evaluating the characteristic of the location uncertainty model comprises comparing the spread against a threshold spread value, and wherein determining the quality of the location estimation algorithm comprises determining whether the spread is below the threshold spread value.

104. The method of claim 103, wherein the threshold spread value is 20 mm or less along any dimension of the location uncertainty model, and wherein determining the quality of the location estimation algorithm comprises determining whether the spread is less than or equal to about 20 mm.

105. The method of claim 102, wherein the location uncertainty model has a mean value, and wherein evaluating the characteristic of the location uncertainty model comprises comparing the mean value against a threshold mean value, and wherein determining the quality of the location estimation algorithm comprises determining whether the mean value is below the threshold mean value.

106. The method of claim 105, wherein the threshold mean value is 3 mm or less, and wherein determining the quality of the location estimation algorithm comprises determining whether the mean value is less than or equal to about 3 mm.

107. The method of claim 102, wherein
generating a first series of target region location data for multiple motion phases based on images of a moving target region comprises deblurring the images of the moving target region to obtain motion-corrected images, and
wherein the method further comprises calculating a deblurring ratio by computing a ratio of an activity concentration of the target region in the motion-corrected images to an activity concentration of the target region in the images of the moving target region and determining the quality of the location estimation algorithm comprises determining whether the deblurring ratio is at least a threshold ratio value.

108. The method of claim 107, wherein the threshold deblurring ratio value is at least 0.8.

109. The method of claim 108, wherein the threshold deblurring ratio value is 0.9 or more.

110. A method for evaluating PET imaging data for radiation delivery, the method comprising:
acquiring PET prescan imaging data of a target region;
initializing a location estimation algorithm using PET prescan images generated from the PET prescan imaging data;
generating a location uncertainty model by applying the initialized location estimation algorithm to the PET imaging data;
determining a difference between the location uncertainty model and an initial location uncertainty model generated using planning PET imaging data; and
generating a notification that indicates whether the determined difference exceeds a threshold.

111. The method of claim 110, wherein if the determined difference does not exceed a threshold, the method further comprises delivering radiation to the target region.

112. The method of claim 111, wherein if the determined difference exceeds a threshold, the method does not comprise delivering radiation to the target region.

113. The method of claim 110, wherein the notification comprises a graphical user interface that depicts the determined difference and the threshold.

114. The method of claim 113, wherein the graphical user interface further comprises a prompt for the user to exit radiation delivery.

115. The method of claim 110, wherein acquiring PET prescan imaging data of the target region comprises detecting positron annihilation emission paths using one or more PET detectors of a radiotherapy system.

116. The method of claim 110, wherein the location estimation algorithm comprises the use of an adaptive correlation filter, and wherein initializing the location estimation algorithm comprises generating a correlation filter using the PET prescan images.

117. The method of claim 110, wherein the location estimation algorithm comprises the use of a maximum likelihood estimation method, and wherein initializing the location estimation algorithm comprises generating a plurality of probability density functions using the PET prescan images.

118. The method of claim 110, wherein the location estimation algorithm is a first location estimation algorithm and the method further comprises initializing a second location estimation algorithm, wherein the first location estimation algorithm comprises the use of an adaptive correlation filter and the second location estimation algorithm comprises the use of a maximum likelihood estimation method, and wherein initializing the location estimation algorithm comprises generating a correlation filter using the PET prescan images and generating a plurality of probability density functions using the PET prescan images.

119. The method of any one of claims 116-117, wherein applying the initialized location estimation algorithm to the PET imaging data generates a first series of target region location data, and wherein generating a location uncertainty model further comprises comparing the first series of target region location data with a second series of target region location data to determine a series of location differences, and generating a location uncertainty cloud from the series of location differences.

120. The method of claim 110, wherein determining the difference between the location uncertainty model and an initial location uncertainty model comprises determining a difference between a spread of each of the location uncertainty models, wherein the spread of a location uncertainty model is 2 times a standard deviation of the location uncertainty model.

121. The method of claim 120, wherein if the difference in the uncertainty model spread exceeds 3 mm in any dimension [x,y,z], the generated notification comprises a visual indicator that the difference in the uncertainty model exceeds the threshold.

122. The method of claim 110, wherein determining the difference between the location uncertainty model and an initial location uncertainty model comprises determining a difference between a mean of each of the location uncertainty models, wherein the mean of a location uncertainty model is an average of error values in location uncertainty model.

123. The method of claim 120, wherein if the difference in the uncertainty model spread exceeds 3 mm, the generated notification comprises a visual indicator that the difference in the uncertainty model exceeds the threshold.

124. The method of claim 110, further comprising calculating a deblurring ratio by deblurring the PET prescan image of the target region to generate motion-corrected images, and computing a ratio of an activity concentration of the target region in the motion-corrected images to an activity concentration of the target region in the PET prescan images, and wherein the method further comprises determining whether the deblurring ratio is at least a threshold ratio value.

125. The method of claim 124, wherein the threshold deblurring ratio value is at least 0.8.

126. The method of claim 125, wherein the threshold deblurring ratio value is 0.9 or more.

127. The method of claim 124, wherein if the deblurring ratio is not at or above the threshold ratio value, the generated notification comprises a visual indicator that the deblurring ratio is below the threshold ratio value.

128. A method for radiotherapy treatment planning, the method comprising:
acquiring PET imaging data of a target region in a treatment position within an acquisition time window;
initializing a location estimation algorithm using PET images reconstructed from the acquired PET imaging data;
calculating a location uncertainty model for the initialized location estimation algorithm; and
generating a radiotherapy treatment plan comprising a set of firing filters iteratively calculated from the location uncertainty model.

129. The method of claim 128, further comprising generating motion-corrected PET images by deblurring the reconstructed PET images.

130. The method of claim 129, wherein deblurring the PET images comprises decomposing the reconstructed PET images into a set of principal components and identifying a dominant principal component from the set of principal components.

131. The method of claim 129, wherein the location uncertainty model comprises a location uncertainty cloud comprising a plurality of target region shifts from an initial target region location.

132. The method of claim 129, wherein generating the radiotherapy treatment plan comprises iterating through candidate firing filters such that when the firing filters are convolved with the location uncertainty model, the radiotherapy treatment plan attains a prescribed radiation dose to the target region while minimizing a dose cost function.

133. The method of any one of claims 128-132, wherein the location estimation algorithm comprises the use of an adaptive correlation filter, and initializing the location estimation algorithm comprises generating a correlation filter using the motion-corrected PET image.

134. The method of any one of claims 128-132, wherein the location estimation algorithm comprises the use of a maximum likelihood estimation method, and initializing the location estimation algorithm comprises generating a plurality of probability density functions using the motion-corrected PET images.

135. The method of claim 128, wherein the location uncertainty model has a spread defined as 2 times a standard deviation of the location uncertainty model, and wherein the method further comprises determining whether the spread is below a threshold spread value, and proceeding to generate the radiotherapy treatment plan if the spread is less than or equal to the threshold spread value.

136. The method of claim 135, wherein the threshold spread value is 20 mm or less along any dimension of the location uncertainty model.

137. The method of claim 128, wherein the location uncertainty model has a mean value, and wherein the method further comprises determining whether the mean value is below the threshold mean value, and proceeding to generate the radiotherapy treatment plan if the mean value is less than or equal to the threshold mean value.

138. The method of claim 137, wherein the threshold mean value is 3 mm or less.

139. The method of claim 129, further comprising calculating a deblurring ratio by computing a ratio of an activity concentration of the target region in the motion-corrected PET images to an activity concentration of the target region in the reconstructed PET images, and wherein the method further comprises determining whether the deblurring ratio is greater than or equal to a threshold ratio value.

140. The method of claim 139, wherein the threshold ratio value is greater than or equal to 0.8.

141. The method of claim 140, wherein the threshold ratio value is greater than or equal to 0.9.

142. The method of claim 128, wherein initializing a location estimation algorithm using PET images reconstructed from the acquired PET imaging data comprises applying filtered back projection to the acquired PET imaging data.

143. The method of claim 128, wherein initializing a location estimation algorithm using PET images reconstructed from the acquired PET imaging data comprises applying single-slice rebinning and filtered back projection to the acquired PET imaging data.

144. The method of claim 128, further comprising aligning the reconstructed PET images with a planning CT image.

145. The method of claim 128, further comprising aligning the reconstructed PET images with a planning MR image.

146. The method of claim 129, further comprising aligning the motion-corrected PET images with a planning CT image.

147. The method of any one of claims 128-129, further comprising computing an activity concentration (AC) of the target region, and proceeding to generate the radiotherapy treatment plan if the activity concentration is within an acceptable range.

148. The method of claim 147, wherein the acceptable range of the activity concentration is greater than or equal to 5 kBq/ml.

149. The method of any one of claims 128-129, further comprising computing a normalized target signal (NTS) of the target region, and proceeding to generate the radiotherapy treatment plan if the normalized target signal is within an acceptable range.

150. The method of claim 149, wherein the acceptable range of the normalized target signal is greater than or equal to 2.

151. The method of claim 150, wherein the acceptable range of the normalized target signal is greater than or equal to 2.7.

152. The method of claim 128, wherein the radiotherapy treatment plan further comprises a planned fluence map that, when delivered, provides a prescribed dose to the target region.
